(19) United States
(12) Patent Application Publication
(10) Pub. No.: US 2025/0020651 A1
(43) Pub. Date: Jan. 16, 2025

(54) PERSONALIZED MODULATION THERAPY FOR CANCER

(71) Applicant: Faeth Therapeutics, Inc., Austin, TX (US)

(72) Inventors: Oliver D.K. MADDOCKS, Glasgow (GB); Petros A. TYRAKIS, Cambridge (GB); Todd YOUNG, San Mateo, CA (US)

(21) Appl. No.: 18/444,504

(22) Filed: Feb. 16, 2024

Related U.S. Application Data
(63) Continuation of application No. PCT/US2022/040588, filed on Aug. 17, 2022.
(60) Provisional application No. 63/234,507, filed on Aug. 18, 2021.
(60) Provisional application No. 63/234,517, filed on Aug. 18, 2021.
(60) Provisional application No. 63/280,792, filed on Nov. 18, 2021.

(51) Int. Cl. G01N 33/574; A61K 31/145; A61K 31/197; A61K 31/198; A61K 31/282; A61K 31/337; A61K 31/405; A61K 31/4172; A61K 31/4745; A61K 31/513; A61K 31/519; A61K 31/5377; A61K 31/7068; A61P 35/00; G01N 33/573

(52) U.S. Cl. CPC G01N 33/57484; A61K 31/145; A61K 31/197; A61K 31/198; A61K 31/282; A61K 31/337; A61K 31/405; A61K 31/4172; A61K 31/4745; A61K 31/513; A61K 31/519; A61K 31/5377; A61K 31/7068; A61P 35/00; G01N 33/573; G01N 2333/904; G01N 2333/91188; G01N 2474/20

(57) ABSTRACT

Disclosed herein are methods and compositions to predict a response of a subject to a nutrient modulation therapy. Restricting the uptake of at least one amino acid by cancer cells can be used to treat or delay cancer. In some embodiments, the nutrition modulation therapy is a dietary product that is substantially devoid of at least one amino acid.

### CROSS-REFERENCE

This application is a continuation of International Application No. PCT/US2022/040588, filed Aug. 17, 2022, which claims the benefit of U.S. Provisional Application No. 63/234,517, filed Aug. 18, 2021; U.S. Provisional Application No. 63/234,507, filed Aug. 18, 2021; and U.S. Provisional Application No. 63/280,792, filed Nov. 18, 2021, each of which is incorporated herein in their entirety.

### BACKGROUND

Compared to normal cells, many cancer cells have evolved differential metabolic and nutritional demands for survival, proliferation, and metastases. Current therapies for the treatment for cancer or other pathologies can be ineffective due to patient-specific factors. Personalized methods and formulations can be developed for therapy of various diseases, including cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, disclosed herein is a method comprising: a) isolating a biological sample from a subject, wherein the subject has a cancer; b) after the isolating, quantifying an amount of a biosynthetic metabolic enzyme; and c) after the quantifying, administering a nutrient modulation therapy to the subject.

In some embodiments, disclosed herein is a method comprising: a) isolating a biological sample from a subject, wherein the subject has a cancer; and b) after the isolating, quantifying an amount of a first biosynthetic metabolic enzyme and a second biosynthetic metabolic enzyme, wherein the amount of the first biosynthetic metabolic enzyme and the second biosynthetic metabolic enzyme predicts a likelihood of the subject displaying a positive response to a nutrient modulation therapy to treat the cancer.

In some embodiments, disclosed herein is a method of treating a cancer in a subject in need thereof, the method comprising: a) administering to the subject a therapeutically effective amount of a dietary product, wherein the dietary product is devoid of at least serine and glycine; and b) administering to the subject to the subject a therapeutically effective amount of a therapeutic agent, wherein a cancer cell of the subject expresses low levels of at least one biosynthetic metabolic enzyme, wherein the at least one biosynthetic metabolic enzyme catalyzes the biosynthesis of serine or glycine, and wherein the administering the dietary product increases an efficacy of the therapeutic agent by at least about 20% as compared to an efficacy of the therapeutic agent in the absence of the dietary product.

In some embodiments, disclosed herein is a method of treating a condition in a subject in need thereof, the method comprising administering a therapeutically effective amount of a dietary product, wherein the dietary product is devoid of at least serine, glycine, and proline. In some embodiments, disclosed herein is a pharmaceutical composition comprising in unit dosage form: a) a plurality of amino acids, wherein the composition is devoid of at least serine, glycine, and proline; and optionally, b) a pharmaceutically acceptable excipient.

### BRIEF DESCRIPTION OF THE DRAWINGS

FIG. 1A shows the correlation between PHGDH enzyme expression and relative growth of cancer cells in serine, glycine, and proline-free media. FIG. 1B shows the correlation between PSAT1 enzyme expression and relative growth of cancer cells in serine, glycine, and proline-free media. FIG. 1C shows the correlation between PHGDH and PSAT 1 enzyme expression and relative growth of cancer cells in serine, glycine, and proline-free media. FIG. 1D shows Pearson Correlation values for combined PHGDH and PSAT1 enzyme expression with cancer cells growth in proline, serine, and proline-free media.

FIG. 2A shows the correlation between PHGDH enzyme expression and relative growth of cancer cells in serine, glycine, and proline-free media. FIG. 2B shows the correlation between PSAT1 enzyme expression and relative growth of cancer cells in serine, glycine, and proline-free media. FIG. 2C shows the correlation between PSPH enzyme expression and relative growth of cancer cells in serine, glycine, and proline-free media. FIG. 2D shows the correlation between PHGDH, PSAT1, and PSPH enzyme expression and relative growth of cancer cells in serine, glycine, and proline-free media. FIG. 2E shows Pearson Correlation values for combined PHGDH, PSAT1, and PSPH enzyme expression with cancer cells growth in proline, serine, and proline-free media.

FIG. 3A shows the correlation between SHMT1 enzyme expression and relative growth of cancer cells in serine, glycine, and proline-free media. FIG. 3B shows the correlation between SHMT2 enzyme expression and relative growth of cancer cells in serine, glycine, and proline-free media.

FIG. 4 rows A-D illustrate the effects of proline/serine/glycine (P/S/G) starvation on pancreatic cancer cells when treated with gemcitabine (row B), paclitaxel (row C), gemcitabine and paclitaxel (row A), or FOLFIRINOX (row D).

FIG. 5A-5E illustrate the effect of P/S/G starvation on pancreatic cancer cells when treated with a disulfiram.

FIG. 6A-6F illustrates the effect of P/S/G starvation on pancreatic cancer cells when treated with serabelisib.

FIG. 7A-7G illustrates correlation between the expression of enzymes involved in serine and glycine synthesis and enzymes which synthesize proline in pancreatic cancer cell lines.

FIG. 8 outlines the stages of the metastatic colorectal cancer clinical trial from baseline/screening to end of study.

### DETAILED DESCRIPTION OF THE INVENTION

Cancer cells acquire metabolic adaptations that support enhanced rates of growth and proliferation of the cancer cells. While the metabolic adaptations help tune metabolism to support higher anabolic output and bolster anti-oxidant defenses, the metabolic adaptations can also decrease metabolic flexibility and impose increased dependence on nutrient uptake and de novo nutrient biosynthesis. Cancer cells have high demands for non-essential amino acids (NEAA), which are precursors for anabolic and anti-oxidant pathways that support cell survival and proliferation. Controlling the levels of a particular amino acid or a combination of amino acids in the diet of a subject can lead to improved treatment of various diseases, including cancer. Modulation of amino acid levels in a cancer cell can control the production of proteins, metabolites, lipids, and nucleic acids that can promote cancer growth and metastasis.

Cancer cells can adapt their metabolism to support growth and survival, leading to various dependencies and vulnerabilities that could be targeted for therapy. Dietary intake and nutrient metabolism are critical in supporting human health. The 20 proteinogenic amino acids can be categorized in two subgroups: essential amino acids and non-essential amino acids. An amino acid is considered “essential” if it cannot be synthesized by the organism and must be derived from exogenous sources. Serine and glycine, which are either obtained by cancer cells from the exogenous environment by cellular uptake, or synthesized de novo from glucose, contribute to a number of important processes, including protein, nucleotide, and lipid synthesis, the generation of antioxidant defense through glutathione and NADPH synthesis and the provision of one-carbon units for the folate cycle and methylation reactions.

Direct mechanisms of resistance to the therapeutic approach of reducing the availability of serine and/or glycine include those that promote increased availability of serine e.g., by serine biosynthesis (at tumor or systemic level) via enhanced expression of the de novo serine synthesis pathway (SSP) enzymes, whose expression can also be promoted by certain oncogenic mutations. Another route for increasing serine availability is the promotion of serine recycling e.g., by mechanisms such as autophagy. Indirect mechanisms of resistance can rely on metabolic adaptations beyond the metabolic pathways directly involved in serine synthesis, for example, downregulating pathways (such as nucleotide synthesis) which consume serine. Combination with other therapeutic agents that target these direct or indirect mechanisms of resistance can improve the ability of serine and glycine starvation to inhibit, for example, tumor growth, tumor initiation, or metastasis. Furthermore, combination with therapeutic agents or interventions that increase the demands of a cancer cell or a tumor for serine or glycine can also sensitize the cancer cell or tumor to other therapeutic agents.

Phosphoglycerate Dehydrogenase (PHGDH) cooperates with serine and glycine depletion to inhibit one-carbon metabolism and cancer growth. In vitro, inhibition of PHGDH combined with serine starvation can lead to a defect in global protein synthesis, which can block the activation of an ATF-4 response and more broadly impacts the protective stress response to amino acid depletion. In vivo, the combination of diet and an inhibitor can exhibit therapeutic efficacy against tumors that are resistant to diet or drug alone, along with reduced one-carbon availability. Inhibition of PHGDH can augment the therapeutic efficacy of a serine-depleted diet. Described herein are compositions and methods for inhibiting PHGDH, the first step in the SSP. Further described herein are compositions and methods for inhibiting PHGDH in combination with compositions devoid of serine and/or glycine.

As a non-essential amino acid, serine can be taken up from the extracellular environment or synthesized de novo by cells using the serine synthesis pathway (SSP). Cancer cells can avidly consume serine and depend on an exogenous source of serine for optimal growth. Some cancer cells can adapt to serine starvation by activating flux through the SSP. Serine is an activator of PKM2, the final step in glycolysis, and decreased PKM2 activity under serine depleted conditions can allow for the diversion of glycolytic intermediates into the SSP. This response is coordinated with an ATF-4 and histone methyltransferase G9A-dependent activation of the three enzymes of the SSP, which can allow most cancer cells to survive and continue to proliferate following serine starvation. The efficacy with which cancer cells can adapt to the loss of exogenous serine depends on several factors. Some cancers acquire an amplification or overexpression of PHGDH—the first step in the SSP—and these cells tend to be less affected by serine starvation. Similarly, activation of oncogenes such as KRAS, MYC, MDM2, and NRF210 can lead to an increase in SSP enzyme expression, also allowing cells to become resistant to depletion of exogenous serine. Conversely, although the p53 tumor suppressor protein can inhibit PHGDH expression, loss of p53 also makes cells more vulnerable to increased ROS that accompanies the switch to de novo serine synthesis, resulting in a decreased survival in serine free medium. Thus, numerous molecular mechanisms can modulate the expression of SSP enzymes, in addition to the tissue of origin can influence the metabolism of a tumor. Given these multiple factors, it is difficult to predict what tumors express high or low amounts of the SSP enzymes and therefore direct testing of enzyme expression in the tumor tissue is desirable.

Proline is required by cancer cells for protein synthesis which supports cellular growth and proliferation. Of particular importance in cancer, proline (along with glycine) is one of the dominant amino acids constituting collagen proteins. Collagen is an important component of the extracellular matrix in the tumor stroma which surrounds cancer cells. The tumor stroma can constitute a large fraction (e.g., up to 90%) of the tumor volume and supports the tumor through metabolic mechanisms and protects the tumor from chemotherapeutics by limiting drug penetration to the cancer cells. Targeting the tumor stroma is an emerging anti-cancer strategy, and limiting proline has the potential to impede collagen synthesis and deposition of extracellular matrix in the tumor stroma.

Controlling specific amino acids cannot be entirely implemented through “normal foods” since any protein-containing food necessarily contains the amino acids that must be eliminated. The methods disclosed herein have two basic components: 1) whole foods that are low in protein; and 2) a formulated supplement that administers a mixture of essential and non-essential amino acids excluding the desired amino acids. In addition to controlling the supply of specific amino acids, oral delivery of purified amino acids can have unique benefits to systemic protein metabolism in patients with cancer. For example, amino acids in free-form (rather than bound within proteins which require digestion) can be better absorbed in patients with compromised digestion.

Disclosed herein is a method comprising: a) isolating a biological sample from a subject, wherein the subject has a cancer; b) after the isolating, quantifying an amount of a biosynthetic metabolic enzyme; and c) after the quantifying, administering a nutrient modulation therapy to the subject.

Disclosed herein is a method comprising: a) isolating a biological sample from a subject, wherein the subject has a cancer; and b) after the isolating, quantifying an amount of a biosynthetic metabolic enzyme, wherein the amount of the biosynthetic metabolic enzyme predicts a likelihood of the subject displaying a positive response to a nutrient modulation therapy to treat the cancer.

Disclosed herein is a method comprising: a) providing to a subject a meal or daily food intake comprising protein in an amount less than the daily recommended protein level for the subject; and b) administering to the subject a therapeutically effective amount of a dietary product, wherein the dietary product is devoid of at least serine and glycine, wherein the dietary product is formulated as a sachet.

Disclosed herein is a method comprising: a) providing to a subject a meal or daily food intake comprising protein in an amount less than the daily recommended protein level for the subject; and b) administering to the subject a therapeutically effective amount of a dietary product, wherein the dietary product is devoid of at least serine, glycine, and proline, wherein the dietary product is formulated as a sachet.

Described herein are methods of treating a cancer in a subject in need thereof, the method comprising: a) modulating the subject's diet to reduce the subject's serine and glycine intake by at least about 50%; and b) administering to the subject a therapeutically effective amount of a therapeutic agent, wherein a cancer cell of the subject expresses low levels of at least one enzyme, wherein the at least one enzyme catalyzes the biosynthesis of serine or glycine, and wherein the administering the dietary product increases an efficacy of the therapeutic agent by at least about 20% as compared to an efficacy of the therapeutic agent in the absence of the dietary product.

Described herein are methods of treating a cancer in a subject in need thereof, the method comprising: a) administering to the subject a therapeutically effective amount of a dietary product, wherein the dietary product is devoid of at least serine and glycine; and b) administering to the subject a therapeutically effective amount of a therapeutic agent, wherein a cancer cell of the subject expresses low levels of at least one enzyme, wherein the at least one enzyme catalyzes the biosynthesis of serine or glycine, and wherein the administering the dietary product increases an efficacy of the therapeutic agent by at least about 20% as compared to an efficacy of the therapeutic agent in the absence of the dietary product.

Disclosed herein is a method of treating a condition in a subject in need thereof, the method comprising administering to the subject a therapeutically effective amount of a dietary product, wherein the dietary product is devoid of at least serine and glycine.

Disclosed herein is a method of treating a condition in a subject in need thereof, the method comprising administering to the subject a therapeutically effective amount of a dietary product, wherein the dietary product is devoid of at least serine, glycine, and proline.

Disclosed herein is a pharmaceutical composition comprising in unit dosage form: a) a plurality of amino acids, wherein the composition is devoid of at least serine, glycine, and proline; and b) a pharmaceutically acceptable excipient.

### Stratified and Personalized Nutrient Modulation Cancer Therapy

Described herein are methods, systems, and compositions to stratify patients into treatment groups and personalize nutrient modulation therapy to treat a condition. In some embodiments, the condition is a cancer. In some embodiments, a method of the disclosure can comprise isolating or labelling a biological sample from a subject.

A biomarker can be any molecule that is measurable and identifiable from other molecules. A biomarker can be associated with a state of a cell, tissue, organ, or subject. Such a state can be a healthy or disease state. A disease state can be a cancer state or a state with a risk of cancer.

In some embodiments, a method of the disclosure can comprise quantifying at least one biomarker in a biological sample from a subject. In some embodiments, a method of the disclosure can comprise quantifying a plurality of biomarkers in a biological sample from a subject. In some embodiments, a method of the disclosure can comprise comparing at least one biomarker from a subject with cancer to at least one biomarker in a plurality of reference samples. In some embodiments, a method of the disclosure can comprise comparing a plurality of biomarkers from a subject with cancer to a plurality of biomarkers in a plurality of reference samples.

In some embodiments, a method of the disclosure can comprise predicting a response of a subject to a nutrient modulation therapy based on the quantification of at least one biomarker in a biological sample from the subject. In some embodiments, a method of the disclosure can comprise predicting a response of a subject to a nutrient modulation therapy based on the quantification of a plurality of biomarkers in a biological sample from the subject. In some embodiments, a method of the disclosure can comprise predicting a response of a subject to a nutrient modulation therapy based on a comparison of at least one biomarker in a biological sample from a subject and at least one reference sample. In some embodiments, a method of the disclosure can comprise predicting a response of a subject to a nutrient modulation therapy based on a comparison of at least one biomarker in a biological sample from a subject and at least one reference sample. In some embodiments, a method of the disclosure can comprise predicting a response of a subject to a nutrient modulation therapy based on a comparison of a plurality of biomarkers in a biological sample from a subject and a plurality of reference samples. In some embodiments, a method of the disclosure can comprise determining the composition of a nutrient modulation therapy based on a quantification of a plurality of biomarkers in a biological sample from a subject.

### Biological Samples

A method and system of the disclosure can utilize a biological sample to stratify a subject into treatment groups for personalized nutrient modulation therapy. In some embodiments, a biological sample is obtained through a tumor biopsy. A biopsy can be a medical or biological test that involves extraction of a cell, tissue, organ, or other biological materials from a body for examination to determine the body state. In some embodiments, a biological sample can comprise a cancer cell. In other embodiments, a biological sample can comprise a non-cancer cell. In some embodiments, a biological sample from a subject with a cancer can comprise a cancer cell from the cancer of the subject. In other embodiments, a biological sample from a subject with a cancer can comprise a non-cancer cell from the subject. In some embodiments, a biological sample from a subject with a cancer can comprise a healthy cell from the subject. In some embodiments, a biological sample can comprise a stroma cell. In some embodiments, a biological sample can comprise a cancer cell and a non-cancer cell. In some embodiments, a biological sample can comprise a cancer cell and a stroma cell. In some embodiments, a biological sample can comprise a homogenous population of cells. In some embodiments, a biological sample can comprise a heterogenous population of cells.

In some embodiments, the biological sample is a tumor biopsy sample. In some embodiments, the biological sample comprises a circulating tumor cell. In some embodiments, the biological sample comprises circulating tumor DNA. In some embodiments, the subject is human.

A cell can be a mammalian cell. A cell can comprise progeny of a cell or cell population. A cell can comprise progeny of a single cell. A tissue can be a group of cells. An organ can be a group of tissues. A tissue or an organ can contain molecules other than a cell.

A cancer cell can be a cell exhibiting a neoplastic cellular phenotype, which can be characterized by one or more of, for example, abnormal cell growth, abnormal cellular proliferation, loss of density dependent growth inhibition, anchorage-independent growth potential, ability to promote tumor growth or development in an immunocompromised non-human animal model, or any appropriate indicator of cellular transformation. A cancer cell can be a tumor cell or a cancerous cell. A cancer cell can also encompass cancer cells of a solid tumor/cancer or a liquid tumor/cancer. A cancer can comprise a cancer cell and can be used interchangeably herein with a tumor. A cancer/tumor can be benign or metastatic.

A solid tumor or a solid cancer can be a tumor without cysts or liquid areas. Solid tumors can comprise brain and other central nervous system tumors (including but not limited to tumors of the meninges, brain, spinal cord, cranial nerves and other parts of central nervous system, e.g., glioblastomas or medulla blastomas); head or neck cancer; breast tumors; circulatory system tumors (including but not limited to heart, mediastinum and pleura, and other intrathoracic organs, vascular tumors and tumor-associated vascular tissue); excretory system tumors (including but not limited to tumors of kidney, renal pelvis, ureter, bladder, other and unspecified urinary organs); gastrointestinal tract tumors (including but not limited to tumors of esophagus, stomach, small intestine, colon, colorectal, rectosigmoid junction, rectum, anus and anal canal, tumors involving the liver and intrahepatic bile ducts, gall bladder, other and unspecified parts of biliary tract, pancreas, other and digestive organs); oral cavity tumors (including but not limited to tumors of lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (including but not limited to tumors of vulva, vagina, Cervix uteri, Corpus uteri, uterus, ovary, and other sites associated with female genital organs, placenta, penis, prostate, testis, and other sites associated with male genital organs); respiratory tract tumors (including but not limited to tumors of nasal cavity and middle ear, accessory sinuses, larynx, trachea, bronchus and lung, e.g., small cell lung cancer or non-small cell lung cancer); skeletal system tumors (including but not limited to tumors of bone and articular cartilage of limbs, bone articular cartilage and other sites); skin tumors (including but not limited to malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites.

A liquid cancer or a liquid tumor can be a cancer cell that is present in body fluids, such as blood, lymph and bone marrow. A liquid cancer can comprise leukemia, myeloma, myelodysplastic syndrome (MDS), and liquid lymphomas. Liquid lymphomas include lymphomas that contain cysts or liquid areas. A liquid cancer may not include a solid tumor, such as a sarcoma, a carcinoma, or a solid lymphoma that does not contain cysts or liquid areas.

In some embodiments, a biological sample can comprise a molecule that originated from a cancer cell. In some embodiments, a molecule originated from a cancer cell can comprise a peptide, protein, nucleic acid, lipid, carbohydrate, any derivatives herein and thereof, or any combinations herein and thereof. A circulating cancer DNA can be a DNA molecule found in the blood circulation that originates from a cancer cell. In some embodiments, a nucleic acid originated from a cancer cell can comprise a circulating cancer DNA. In some embodiments, a circulating cancer DNA can be separated from other types of DNA. In some embodiments, a circulating cancer DNA can be enriched or purified.

In some embodiments, an enriched or purified population of circulating cancer DNA can comprise at least about 70%, at least about 75%, at least about 80%, at least about 85%, at least about 90%, at least about 95%, at least about 96%, at least about 97%, at least about 98%, or at least about 99% of circulating cancer DNA molecules in a population of molecules. In some embodiments, an enriched or purified population of circulating cancer DNA can comprise from about 70% to about 80%, from about 75% to about 85%, from about 80% to about 90%, from about 85% to about 94%, from about 90% to about 95%, from about 91% to about 96%, from about 92% to about 97%, from about 93% to about 98%, from about 94% to about 99%, or from about 95% to about 100% circulating cancer DNA molecules in a population of molecules. In some embodiments, a molecule originated from a cancer cell can further comprise an organic or inorganic molecule. In some embodiments, a molecule from a biological sample can comprise any biomarkers described herein.

In some embodiments, a biological sample can comprise a cell or a molecule. In some embodiments, a cell or a molecule of a biological sample can be separated from another cell or molecule.

In some embodiments, a biological sample can comprise about 1×10¹ cells, about 5×10¹ cells, about 9×10¹ cells, about 1×10² cells, about 5×10² cells, about 9×10² cells, about 1×10³ cells, about 5×10³ cells, about 9×10³ cells, about 1×10⁴ cells, about 5×10⁴ cells, about 9×10⁴ cells, about 1×10⁵ cells, about 5×10⁵ cells, about 9×10⁵ cells, about 1×10⁶ cells, about 5×10⁶ cells, about 9×10⁶ cells, about 1×10⁷ cells, about 5×10⁷ cells, about 9×10⁷ cells, about 1×10⁸ cells, about 5×10⁸ cells, about 9×10⁸ cells, about 1×10⁹ cells, about 5×10⁹ cells, about 9×10⁹ cells, about 1×10¹⁰ cells, about 5×10¹⁰ cells, about 9×10¹⁰ cells, about 1×10¹¹ cells, about 5×10¹¹ cells, about 9×10¹¹ cells, about 1×10¹² cells, about 5×10¹² cells, about 9×10¹² cells, about 1×10¹³ cells, about 5×10¹³ cells, about 9×10¹³ cells, about 1×10¹⁴ cells, about 5×10¹⁴ cells, about 9×10¹⁴ cells, about 1×10¹⁵ cells, or more cells. In some embodiments, a biological sample can comprise from about 1×10¹ to about 1×10² cells, from about 5×10¹ to about 5×10² cells, from about 1×10² to about 1×10³ cells, from about 5×10² to about 5×10³ cells, from about 1×10³ to about 1×10⁴ cells, from about 5×10³ to about 5×10⁴ cells, from about 1×10⁴ to about 1×10⁵ cells, from about 5×10⁴ to about 5×10⁵ cells, from about 1×10⁵ to about 1×10⁶ cells, from about 5×10⁵ to about 5×10⁶ cells, from about 1×10⁶ to about 1×10⁷ cells, from about 5×10⁶ to about 5×10⁷ cells, from about 1×10⁷ to about 1×10⁸ cells, from about 5×10⁷ to about 5×10⁸ cells, from about 1×10⁸ to about 1×10⁹ cells, from about 5×10⁸ to about 5×10⁹ cells, from about 1×10⁹ to about 1×10¹⁰ cells, from about 5×10⁹ to about 5×10¹⁰ cells, from about 1×10¹⁰ to about 1×10¹¹ cells, from about 5×10¹⁰ to about 5×10¹¹ cells, from about 1×10¹¹ to about 1×10¹² cells, from about 5×10¹¹ to about 5×10¹² cells, from about 1×10¹² to about 1×10¹³ cells, from about 5×10¹² to about 5×10¹³ cells, from about 1×10¹³ to about 1×10¹⁴ cells, from about 5×10¹³ to about 5×10¹⁴ cells, or from about 1×10¹⁴ to about 1× 10¹⁵ cells.

In some embodiments, isolating a biological sample can comprise extracting the biological sample from a subject with cancer. In some embodiments, an extraction can comprise harvesting a tumor or cancer cell from a biopsy of tissues from a subject with cancer. In some embodiments, an extraction can comprise harvesting a tumor or cancer cell from a biopsy of a site of tumor or cancer from a subject with cancer. In some embodiments, an extraction can comprise harvesting a tumor or cancer cell from a biopsy of circulating tumor or cancer cells from a subject with cancer. In some embodiments, a biopsy can comprise a bone biopsy, a bone marrow biopsy, a breast biopsy, a gastrointestinal biopsy, a lung biopsy, a liver biopsy, a prostate biopsy, a nervous system biopsy, a urogenital biopsy, a lymph node biopsy, a muscle biopsy, a skin biopsy, a blood biopsy, a body fluid biopsy, a cardiac biopsy, an endometrial biopsy, an open biopsy, a sentinel lymph node biopsy, any derivatives herein and thereof, or any combinations herein and thereof. In some embodiments, a biopsy can comprise a fine needle aspiration biopsy, a core needle biopsy, a vacuum-assisted biopsy, an excisional biopsy, a shave biopsy, a punch biopsy, an endoscopic biopsy, a laparoscopic biopsy, a bone marrow aspiration biopsy, a liquid biopsy, any derivatives herein and thereof, or any combinations herein and thereof. In other embodiments, a biopsy can comprise an incisional biopsy or an excisional biopsy.

In some embodiments, a method can comprise isolating a biological sample from a subject with cancer. In some embodiments, isolating a biological sample from a subject with cancer can comprise removing the biological sample from the subject with cancer. In some embodiments, a biological sample can comprise a solid sample or a liquid sample. In some embodiments, a liquid sample can comprise a body fluid. In some embodiments, the body fluid is an intracellular body fluid or extracellular body fluid. In some embodiments, the extracellular body fluid is an intravascular fluid, interstitial fluid, lymphatic fluid, or transcellular fluid. In some embodiments, a body fluid can comprise blood, tears, phlegm, mucus, semen, sweat, saliva, or urine. In some embodiments, a solid biological sample can comprise an extracted cell or groups of cells described herein and thereof. In some embodiments, a solid biological sample can also comprise stool, debris, earwax, or undigested food.

In some embodiments, a biological sample comprising a tumor or cancer cell extracted from a subject with cancer can be cultured, grown, or maintained with viability in vitro, ex vivo, or outside of the subject or a host organism. In some embodiments, a biological sample comprising a tumor or cancer cell extracted from a subject with cancer may not be cultured, grown, or maintained with viability in vitro, ex vivo, or outside of the subject or a host organism. In some embodiments, a biological sample can be lysed or processed after being isolated.

### Biomarkers

In some embodiments, a method can comprise quantifying a biomarker. In some embodiments, the biomarker is a peptide, protein, nucleic acid, metabolite, or cell. In some embodiments, the biomarker can comprise an organic or inorganic molecule. In some embodiments, a nucleic acid biomarker can comprise a DNA or RNA. In some embodiments, a DNA biomarker can comprise a gene. In some embodiments, a DNA biomarker can also comprise a fragment or sequence of a gene. In some embodiments, a DNA biomarker can comprise a circulating cancer DNA. In some embodiments, a DNA biomarker can comprise a DNA from a genome. In some embodiments, a DNA biomarker can comprise a nuclear gene, gene fragment, or genetic sequence. In some embodiments, a DNA biomarker can comprise a mitochondrial gene, gene fragment, or genetic sequence. In some embodiments, a DNA biomarker can comprise a viral gene, gene fragment, or genetic sequence. In other embodiments, a DNA biomarker can also comprise a nuclear, mitochondrial, or plasmid DNA from a microbe in a subject. In some embodiments, the biomarker can comprise any microbiome organism.

In some embodiments, a DNA biomarker can comprise the copy number of the DNA biomarker. In other embodiments, a DNA biomarker can comprise the sequence identity of the DNA biomarker. In some embodiments, a DNA biomarker can also comprise the chromosomal location of the DNA biomarker. In some embodiments, a DNA biomarker can comprise an epigenetic marker, for example, DNA methylation or acetylation. In some embodiments, a DNA biomarker can comprise a gene copy number analysis.

In some embodiments, a nucleic acid biomarker can comprise an RNA. In some embodiments, the RNA biomarker can comprise an mRNA, a tRNA, a lncRNA, a lincRNA, a miRNA, an rRNA, an snRNA, a microRNA, a siRNA, a piRNA, a snoRNA, a snRNA, an exRNA, a scaRNA, a YRNA, an eRNA, or an hnRNA. In other embodiments, an mRNA biomarker can also comprise a pre-mRNA or mRNA.

In some embodiments, a biomarker can comprise a protein. In some embodiments, a biomarker can comprise a peptide. In some embodiments, the protein or peptide is an intracellular protein or peptide. In some embodiments, the protein or peptide is an extracellular protein or peptide.

In some embodiments, a biomarker can comprise a gene or DNA sequence encoding the biomarker. In some embodiments, a biomarker can comprise a transcript or RNA of a gene or DNA sequence encoding the biomarker. In some embodiments, a biomarker can comprise a protein encoded by a gene or DNA sequence encoding the biomarker.

In some embodiments, a biomarker can comprise any protein, nucleic acid, or metabolite associated with an amino acid metabolism pathway. In some embodiments, a protein, nucleic acid, or metabolite associated with an amino acid metabolism pathway can comprise any protein, nucleic acid, or metabolite that is involved in the biosynthesis, uptake, chemical conversion, transport, degradation, secretion, storage, absorption, or recycling of an amino acid or its derivative. In some embodiments, the amino acid metabolism pathway being monitored with a biomarker can comprise a biomarker of an essential amino acid, a conditionally essential amino acid, a nonessential amino acid, or any combinations thereof. In some embodiments, the essential amino acid is not produced from other compounds by the human body. In some embodiments, an essential amino acid can be taken in as food. In some embodiments, the essential amino acid is histidine, isoleucine, leucine, lysine, methionine, phenylalanine, threonine, tryptophan, or valine. In some embodiments, a conditionally essential amino acid is the amino acid that is essential for certain conditions (e.g., in a cancer cell or in a tumor). In some embodiments, the conditionally essential amino acid is arginine, cysteine, glutamine, glycine, proline, or tryptophan. In some embodiments, the non-essential amino acid is alanine, aspartic acid, asparagine, glutamic acid, or serine. In some embodiments, the amino acid metabolism pathway being monitored with a biomarker can comprise a biomarker for at least one of histidine, isoleucine, leucine, lysine, methionine, phenylalanine, threonine, tryptophan, valine, arginine, cysteine, glutamine, glycine, proline, tryptophan, alanine, aspartic acid, asparagine, glutamic acid, or serine. In some embodiments, the amino acid metabolism pathway being monitored with a biomarker can comprise a biomarker for at least two of histidine, isoleucine, leucine, lysine, methionine, phenylalanine, threonine, tryptophan, valine, arginine, cysteine, glutamine, glycine, proline, tryptophan, alanine, aspartic acid, asparagine, glutamic acid, or serine. In some embodiments, the amino acid metabolism pathway being monitored with a biomarker can comprise a biomarker for at least three, at least four, at least five or more of histidine, isoleucine, leucine, lysine, methionine, phenylalanine, threonine, tryptophan, valine, arginine, cysteine, glutamine, glycine, proline, tryptophan, alanine, aspartic acid, asparagine, glutamic acid, or serine. In some embodiments, the amino acid metabolism pathway being monitored with a biomarker can comprise a biomarker for glycine. In some embodiments, the amino acid metabolism pathway being monitored with a biomarker can comprise a biomarker for serine. In some embodiments, the amino acid metabolism pathway being monitored with a biomarker can comprise a biomarker for proline.

In some embodiments, a biomarker can comprise an amino acid biosynthetic enzyme. In some embodiments, an amino acid biosynthetic enzyme biomarker is a serine synthesis enzyme. In some embodiments, the serine synthesis enzyme biomarker can comprise a biomarker for PHGDH, PSPH, or PSAT1. In some embodiments, a biomarker can be an amino acid metabolizing enzyme. In some embodiments, an amino acid metabolizing enzyme biomarker can be methionine adenosyltransferase (MAT). In some embodiments, a biomarker can be an amino acid degradation enzyme. In some embodiments, an amino acid degradation enzyme can be proline oxidase or PRODH. In some embodiments, a biomarker can comprise an amino acid metabolism sensor. In other embodiments, a biomarker can comprise a transcriptional regulator. In some embodiments, an amino acid metabolism sensor or a biomarker can comprise GCN2 or ATF4. In some embodiments, a biomarker can comprise an amino acid transporter. In some embodiments, an amino acid transporter can comprise SLC7A11. In some embodiments, a biomarker can comprise an anabolic enzyme. An anabolic enzyme, in some embodiments, can use amino acids or amino acid derived metabolites as precursors. In some embodiments, an anabolic enzyme biomarker can comprise glutamate cysteine ligase (GCS). In some embodiments, a biomarker can comprise an amino acid salvage/recycling enzyme. In some embodiments, an amino acid salvage/recycling enzyme biomarker can comprise methylthioadenosine (MTAP). In some embodiments, an amino acid salvage/recycling enzyme biomarker can also comprise PGAM, PKM2, SHMT1, or SHMT2.

In some embodiments, a biomarker can comprise a general marker of amino acid metabolism. Such a general marker of amino acid metabolism can comprise GCN2 or ATF4. In some embodiments, a biomarker can comprise a marker for proline enzymes. In some embodiments, a marker for proline enzymes can comprise ALDH18A1, PYCR1, PYCR2, PYCR3, or PRODH. In some embodiments, a biomarker can comprise an enzyme associated with cysteine/cystine metabolism. In some embodiments, an enzyme associated with cysteine/cystine metabolism can comprise CSE, CBS, methyltransferases, AHCY, MTAP, AMD1, SSAT, PAOX, SMOX, or GPX4.

In some embodiments, a biomarker can comprise an enzyme involved in the synthesis or processing of a nutrient. In some embodiments, the nutrient is an amino acid, a lipid, a fatty acid, a vitamin, or a carbohydrate. In some embodiments, a biomarker can comprise a metabolite. In some embodiments, the metabolite can comprise a phospholipid, carbohydrate, organic or inorganic compound. In some embodiments, the metabolite is histidine, isoleucine, leucine, lysine, methionine, phenylalanine, threonine, tryptophan, valine, arginine, cysteine, glutamine, glycine, proline, tryptophan, alanine, aspartic acid, asparagine, glutamic acid, or serine, metabolites which are intermediates in amino acid metabolism, e.g., for serine synthesis: 3-phosphoserine, 3-phosphoglycerate, or 3-phosphohydroxypyruvate.

In some embodiments, a biomarker can comprise an enzyme involved in the synthesis or processing of an amino acid. In some embodiments, the biomarker is an enzyme involved in the synthesis or processing of serine. In some embodiments, the biomarker is a precursor of serine. In some embodiments, the biomarker is 3-phospho-D-glycerate (PHGDH), 3-phosphonooxy pyruvate (PSAT1), phosphoserine, or glycine. In some embodiments, the biomarker is 3-phosphoglycerate, phosphohydroxypyruvate, phosphoserine, or glycine. In some embodiments, the biomarker is phosphoglycerate dehydrogenase, phosphoserine aminotransferase, or phosphoserine phosphatase. In some embodiments, the biomarker is phosphoglycerate dehydrogenase (PHGDH). In some embodiments, the biomarker is phosphoserine aminotransferase (PSAT1). In some embodiments, the biomarker is phosphoserine phosphatase (PSPH).

In some embodiments, the biomarker is an enzyme involved in the synthesis or processing of glycine. In some embodiments, the biomarker is a precursor of glycine. In some embodiments, the biomarker is serine, glyoxylate, betaine, dimethylglycine, or sarcosine. In some embodiments, the biomarker is serine hydroxymethyl transferase 1 (SHMT1), serine hydroxymethyl transferase 2 (SHMT2), alanine-glyoxylate aminotransferase (AGXT), betaine-homocysteine S-methyltransferase (BHMT), dimethylglycine dehydrogenase (DMGD), or sarcosine dehydrogenase (SARDH).

In some embodiments, the biomarker is an enzyme involved in the synthesis or processing of cysteine. In some embodiments, the biomarker is a precursor of cysteine. In some embodiments, the biomarker is homocysteine, cystathionine, serine, methionine, S-adenosyl-L-homocysteine, S-adenosyl-L-methionine. In some embodiments, the biomarker is cystathionine beta synthase (CBS), cystathionine gamma-lyase (CTH), methionine adenosyltransferase 2B (MAT2B), DNA methyltransferase 1 (DNMT1), or adenosylhomocysteinase (AHCYL1).

In some embodiments, the biomarker is an enzyme involved in the synthesis or processing of glutamate. In some embodiments, the biomarker is a precursor of glutamate. In some embodiments, the biomarker is 2-oxoglutarate, glutamine, or 5-oxoproline. In some embodiments, the biomarker is glutaminase (GLS), glutamate dehydrogenase (GLDH), or 5-oxoprolinase (OXP1).

In some embodiments, the biomarker is an enzyme involved in the synthesis or processing of glutamine. In some embodiments, the biomarker is a precursor of glutamine. In some embodiments, the biomarker is glutamate. In some embodiments, the biomarker is glutamine synthetase (GS).

In some embodiments, the biomarker is an enzyme involved in the synthesis or processing of proline. In some embodiments, the biomarker is a precursor of proline. In some embodiments, the biomarker is glutamate, L-glutamyl-5-phosphate, L-glutamate-5-semialdehyde, (S)-1-pyrroline-5-carboxylate, or L-ornithine. In some embodiments, the biomarker is ornithine aminotransferase (OAT), pyrroline-5-carboxylate reductase 1 (PYCR1), pyrroline-5-carboxylate reductase 2 (PYCR2), or glutamate-5-semialdehyde dehydrogenase (ALDH18A1).

In some embodiments, the biomarker is an enzyme involved in the synthesis or processing of arginine. In some embodiments, the biomarker is a precursor of arginine. In some embodiments, the biomarker is glutamate, N-acetyl-L-glutamate, N-acetyl glutamyl-phosphate, N-acetyl-L-glutamate 5 semialdehyde, N-acetyl-L-ornithine, L-ornithine, L-citrulline, L-argininosuccinate, N-acetyl-L-citrulline, or carbamoyl phosphate. In some embodiments, the biomarker is argininosuccinate synthase (ASS1), argininosuccinate lyase (ASL), N-acetylglutamate synthase (NAGS), ornithine transcarbamylase (OTC).

In some embodiments, the biomarker is an enzyme involved in the synthesis or processing of aspartate. In some embodiments, the biomarker is a precursor of aspartate. In some embodiments, the biomarker is oxaloacetate or asparagine. In some embodiments, the biomarker is aspartate aminotransferase 1 (GOT1), aspartate aminotransferase 2 (GOT2), or asparaginase (ASPG).

In some embodiments, the biomarker is an enzyme involved in the synthesis or processing of asparagine. In some embodiments, the biomarker is a precursor of asparagine. In some embodiments, the biomarker is aspartate. In some embodiments, the biomarker is asparagine synthetase.

In some embodiments, the biomarker is an enzyme involved in the synthesis or processing of tyrosine. In some embodiments, the biomarker is a precursor of tyrosine. In some embodiments, the biomarker is phenylalanine, tetrahydrobiopterin (cofactor), 4a-hydroxytetrahydrobiopterin (cofactor), or q-dihydrobiopterin (cofactor). In some embodiments, the biomarker is phenylalanine hydroxylase (PAH), quinoid dihydropteridine reductase (QDPR), or pterin-4-alpha-carbinoamine dehydratase (PCBD1).

In some embodiments, the biomarker is an enzyme involved in the synthesis or processing of alanine. In some embodiments, the biomarker is a precursor of alanine. In some embodiments, the biomarker is pyruvate. In some embodiments, the biomarker is alanine transaminase (ALT1, GPT, GTP2).

In some embodiments, the biomarker is an enzyme involved in the synthesis or processing of selenocysteine. In some embodiments, the biomarker is a precursor of selenocysteine. In some embodiments, the biomarker is hydrogen selenide, selenophosphate, or serine. In some embodiments, the biomarker is selenophosphate synthetase 1 (SEPHS1), selenophosphate synthetase 2 (SEPHS2), selenocysteinyl-tRNA synthase (SEPSECS), seryl-tRNA-synthetase (SARS), or O-phosphoseryl-tRNA-kinase (PSTK).

In some embodiments, a biomarker can be an artificial biomarker marker. In some embodiments, an artificial biomarker may not originate from a biological sample of a subject. In some embodiments, an artificial biomarker may not originate from a cell or a subject. In some embodiments, an artificial biomarker can comprise a tracer. In some embodiments, a tracer can be detectable. In some embodiments, a tracer can be detected by a physical or chemical method. In some embodiments, a tracer detected using a physical method can be a radioactive tracer. In some embodiments, the radioactive tracer can comprise tritium, ¹¹C, ¹⁴C, ³H, ¹³N, ¹⁵O, ¹⁸F, ³²P, ³³P, ³²S, ³⁵S, ³⁵Cl, ⁹⁹Tc, ⁹⁹Mo, ^{99m}Tc, ⁶⁷G, ¹²³I, ¹²³Te, ¹²⁴Xe, ¹²⁵I, or ¹²⁹I. In other embodiments, a radioactive tracer can comprise alpha, beta, or gamma radiation. In other embodiments, the tracer is an optical tracer. In some embodiments, the optical tracer can comprise a chromophore. In other embodiments, the optical tracer can comprise a fluorophore. In some embodiments, a tracer detected using a chemical method can trace a chemical reaction between a tracer and a chemical detection agent. In some embodiments, the chemical detection agent can comprise any molecule disclosed herein.

In some embodiments, a tracer can be taken up by a biological sample. In some embodiments, a tracer can be taken up by a cell or a subject. In some embodiments, a tracer can be incorporated in a molecule so that the tracer is taken up by a biological sample, cell, or subject. In some embodiments, a tracer can be incorporated into an amino acid.

In some embodiments, an artificial biomarker can comprise a genetic reporter. In some embodiments, a genetic reporter can comprise a transcriptional, post-transcriptional, translational, or post-translational reporter. In some embodiments, a genetic reporter can comprise a genetic sequence encoding a fluorophore, for example, a fluorescent protein. In other embodiments, a genetic reporter can comprise a selection marker. In other embodiments, a genetic reporter can also comprise a genetic sequence encoding a protein that emits luminance. In some embodiments, the selection marker is a nutritional marker. In other embodiments, a selection marker can confer drug resistance or drug sensitivity to a cell, a subject, or a biological sample.

### Quantification of Biomarkers

The methods of the disclosure can quantify a biomarker of a biological sample. In some embodiments, quantifying a biomarker can comprise measuring the absolute abundance of a biomarker. In some embodiments, quantifying a biomarker can comprise measuring the relative abundance of a biomarker. In some embodiments, relative abundance of a biomarker can be expressed using the abundance of another molecule. In some embodiments, quantifying a biomarker can comprise identifying the number of molecules of the biomarker. In other embodiments, quantifying a biomarker can comprise identifying weight of a biomarker. In some embodiments, quantifying a biomarker can comprise measuring the distribution of the biomarker. In some embodiments, quantifying a biomarker can comprise quantitative measurement of a biomarker. In other embodiments, quantifying a biomarker can comprise qualitative measurement of a biomarker. In some embodiments, quantifying a biomarker can comprise any combinations of quantifying described herein and thereof.

In some embodiments, quantifying a biomarker can comprise an assay comprising at least one of immunohistochemistry, immunofluorescence, proteomic mass spectrometry, western blot, enzyme-linked immunosorbent assay (ELISA), a genetic reporter, reverse transcription polymerase chain reaction (RT-PCR), quantitative RT-PCR (QPCR), digital PCR (dPCR), in-situ hybridization, Sanger sequencing, high throughput sequencing, or microarray analysis. In some embodiments, quantifying a biomarker can comprise an assay comprising at least two of immunohistochemistry, immunofluorescence, proteomic mass spectrometry, western blot, enzyme-linked immunosorbent assay (ELISA), a genetic reporter, reverse transcription polymerase chain reaction (RT-PCR), quantitative RT-PCR (QPCR), digital PCR (dPCR), in-situ hybridization, Sanger sequencing, high throughput sequencing, or microarray analysis. In some embodiments, quantifying a biomarker can comprise an assay comprising at least two, at least three, at least four, or at least five of immunohistochemistry, immunofluorescence, proteomic mass spectrometry, western blot, enzyme-linked immunosorbent assay (ELISA), a genetic reporter, reverse transcription polymerase chain reaction (RT-PCR), quantitative RT-PCR (QPCR), digital PCR (dPCR), in-situ hybridization, Sanger sequencing, high throughput sequencing, or microarray analysis. In some embodiments, quantifying a biomarker can comprise immunohistochemistry. In some embodiments, quantifying a biomarker can comprise immunofluorescence. In some embodiments, quantifying a biomarker can comprise proteomic mass spectrometry. In some embodiments, quantifying a biomarker can comprise western blot analysis. In some embodiments, quantifying a biomarker can comprise enzyme-linked immunosorbent assay (ELISA) analysis. In some embodiments, quantifying a biomarker can comprise a use of a genetic reporter. In some embodiments, quantifying a biomarker can comprise reverse transcription polymerase chain reaction (RT-PCR). In some embodiments, quantifying a biomarker can comprise quantitative RT-PCR (QPCR). In some embodiments, quantifying a biomarker can comprise digital PCR (dPCR). In some embodiments, quantifying a biomarker can comprise in-situ hybridization. In some embodiments, quantifying a biomarker can comprise Sanger sequencing. In some embodiments, quantifying a biomarker can comprise high throughput sequencing. In some embodiments, quantifying a biomarker can comprise microarray.

In some embodiments, the quantifying comprises analysis by a plurality of assays. In some embodiments, the quantifying comprises analysis by immunohistochemistry, immunofluorescence, proteomic mass spectrometry, western blot, enzyme-linked immunosorbent assay (ELISA), a genetic reporter, reverse transcription polymerase chain reaction (RT-PCR), quantitative RT-PCR (QPCR), digital PCR (dPCR), in-situ hybridization, RNA-Seq, Sanger sequencing, high throughput sequencing, or microarray. In some embodiments, the quantifying is by immunohistochemistry.

In some embodiments, quantifying a biomarker can comprise measuring the protein or peptide expression level of the biomarker. In some embodiments, measuring the protein or peptide expression level of a biomarker (e.g., a protein or a peptide) can comprise immunohistochemistry, immunofluorescence, or proteomic mass spectrometry. In some embodiments, measuring the protein or peptide expression level of a biomarker can comprise western blot or enzyme-linked immunosorbent assay (ELISA). In other embodiments, measuring the protein or peptide expression level of a biomarker can comprise a genetic reporter.

In some embodiments, quantifying a biomarker can comprise measuring the RNA level of a biomarker. In some embodiments, measuring the RNA level of a biomarker (e.g., a transcript/mRNA or other RNA species described herein) can comprise reverse transcription polymerase chain reaction (RT-PCR), quantitative RT-PCR (QPCR), digital PCR (dPCR), in-situ hybridization, Sanger sequencing, high throughput sequencing, or microarray analysis. In some embodiments, in-situ hybridization can comprise a fluorescent in-situ hybridization to measure RNA at a single molecule level.

In some embodiments, quantifying a biomarker can comprise measuring the DNA level of a biomarker. In some embodiments, measuring the DNA level of a biomarker (e.g., a gene or gene sequence) can comprise PCR, quantitative RT-PCR (QPCR), digital PCR (dPCR), Sanger sequencing, high throughput sequencing, or microarray analysis. In some embodiments, measuring the DNA level of a gene or gene sequence can comprise measuring the copy number variations of a gene or gene sequence.

In some embodiments, high throughput sequencing can be used to sequence DNA or RNA in a rapid and cost-effective manner. In some embodiments, high throughput sequencing can be used to sequence multiple DNA or RNA molecules in parallel. In some embodiments, high throughput sequencing can be used to sequence hundreds of millions of DNA or RNA molecules to be sequenced in parallel. In some embodiments, high throughput sequencing can comprise sequencing by ligation. In other embodiments, high throughout sequencing can comprise sequencing by synthesis. In other embodiments, high throughout sequencing can comprise detecting light or hydrogen ions during nucleotide incorporation. In some embodiments, high throughput sequencing can comprise combinatorial probe anchor synthesis. In other embodiments, high throughout sequencing can comprise sequencing by nanopore sequencing. In some embodiments, high throughput sequencing can comprise a light-based sequencing technology.

In some embodiments, quantifying a biomarker can comprise measuring the metabolite level of a biomarker. In some embodiments, measuring the metabolite level of a biomarker (e.g., a metabolite described herein) can comprise detecting or measuring a metabolite by liquid chromatography coupled mass spectrometry. In some embodiments, a radioactive tracer can be measured by a PET scanner, a SPECT scanner, a CT scan, or a technetium scanner. In some embodiments, an activity of a genetic reporter can be quantified. For example, if a genetic reporter comprises a fluorescent protein, the genetic reporter can be quantified by measuring the fluorescent level or protein expression level of the fluorescent protein. In another example, if a genetic reporter encodes a product that confers drug resistance or drug sensitivity to a drug, the genetic reporter can be quantified by measuring the drug resistance or drug sensitivity level of a cell comprising the genetic reporter.

In some embodiments, a biomarker can be quantified using an empirical scoring system. In some embodiments, a quantification result can be evaluated by a semiquantitative approach. In some embodiments, immunohistochemistry results can be assigned an H-score (or “histo” score) to tumor samples. First, membrane staining intensity (0, 1+, 2+, or 3+) can be determined for each cell in a fixed field. The cells can be categorized as negative (0), weak (1+), moderate (2+), and strongly (3+) stained membranes. The H-score can be based on a predominant staining intensity and include the sum of individual H-scores for each intensity level seen. In some embodiments, the percentage of cells at each staining intensity level can be calculated, and an H-score can be assigned using the following formula:

$$[ 1 \times ( \% cells 1 + ) + 2 \times ( \% cells 2 + ) + 3 \times ( \% cells 3 + ) ]$$

The final score, ranging from 0 to 300, gives more relative weight to higher-intensity membrane staining in a given tumor sample. The sample can then be considered positive or negative on the basis of a specific discriminatory threshold.

In some embodiments, a method of the disclosure can quantify 1, 2, 3, 4, 5, 6, 7, 8, 9, or 10 biomarkers, for example, biosynthetic metabolic enzymes. In some embodiments, a method of the disclosure can quantify 2 biomarkers. In some embodiments, a method of the disclosure can quantify 3 biomarkers. In some embodiments, a method of the disclosure can quantify 4 biomarkers.

In some embodiments, a method of the disclosure can quantify at least 2 biomarkers, and the biomarkers can be weighted in predicting a response of a subject to a nutrient modulation therapy. In some embodiments, a method of the disclosure can quantify a first biomarker and a second biomarker, and the first biomarker and second biomarker can be weighted at a ratio of about 1:1, about 1:2, about 1:3, about 1:4, about 1:5, about 1:6, about 1:7, about 1:8, about 1:9, about 1:10, about 10:1, about 9:1, about 8:1, about 7:1, about 6:1, about 5:1, about 4:1, about 3:1, or about 2:1. In some embodiments, the first biomarker and the second biomarker can be weighted at a ratio of about 1:1. In some embodiments, the first biomarker and the second biomarker can be weighted at a ratio of about 2:1. In some embodiments, the first biomarker and the second biomarker can be weighted at a ratio of about 5:1.

In some embodiments, the at least one biosynthetic metabolic enzyme catalyzes the biosynthesis of serine. In some embodiments, the at least one biosynthetic metabolic enzyme is phosphoglycerate dehydrogenase (PHGDH). In some embodiments, the subject expresses PHGDH at a level that is lower than a PHGDH level of a reference sample, wherein the reference sample does not comprise a cancer cell. In some embodiments, the at least one biosynthetic metabolic enzyme is phosphoserine aminotransferase 1 (PSAT1). In some embodiments, the subject expresses PSAT1 at a level that is lower than a PSAT1 level of a reference sample, wherein the reference sample does not comprise a cancer cell. In some embodiments, the at least one biosynthetic metabolic enzyme is phosphoserine phosphatase (PSPH). In some embodiments, the subject expresses PSPH at a level that is lower than a PSPH level of a reference sample, wherein the reference sample does not comprise a cancer cell. In some embodiments, the at least one biosynthetic metabolic enzyme catalyzes the biosynthesis of glycine. In some embodiments, the at least one biosynthetic metabolic enzyme is serine hydroxy methyltransferase 1 (SHMT1). In some embodiments, the subject expresses SHMT1 at a level that is lower than a SHMT1 level of a reference sample, wherein the reference sample does not comprise a cancer cell. In some embodiments, the at least one biosynthetic metabolic enzyme is serine hydroxy methyltransferase 2 (SHMT2). In some embodiments, the subject expresses SHMT2 at a level that is lower than a SHMT2 level of a reference sample, wherein the reference sample does not comprise a cancer cell. In some embodiments, the at least one biosynthetic metabolic enzyme is glycine synthase. In some embodiments, the subject expresses glycine synthase at a level that is less than a glycine synthase level of a reference sample, wherein the reference sample does not comprise a cancer cell.

In some embodiments, the biosynthetic metabolic enzyme is involved in a nutrient biosynthesis. In some embodiments, the biosynthetic metabolic enzyme is involved in a nutrient processing. In some embodiments, the nutrient is a fatty acid. In some embodiments, the nutrient is a lipid. In some embodiments, the nutrient is an amino acid.

In some embodiments, the biosynthetic metabolic enzyme is PHGDH. In some embodiments, the biosynthetic metabolic enzyme is PSAT1.

### Predicting a Response of a Subject to Nutrient Modulation

A method disclosed herein can be used to predict a response of a subject to a nutrient modulation therapy based on a quantification of a plurality of biomarkers in a biological sample from the subject. A nutrient modulation, a nutrient therapy, or a diet restriction can be a diet that is restricted for at least one nutrient. In some embodiments, predicting a response of a subject to a nutrient modulation therapy can comprise a quantification of a plurality of biomarkers in a biological sample from the subject. In some embodiments, an absence of a biomarker can predict a positive response to a nutrient modulation therapy. In some embodiments, an absence of a biomarker for an amino acid metabolism pathway in a subject, such as any of those described herein, can predict a positive response of the subject to a nutrient modulation therapy, wherein the nutrient modulation limits the amount of the amino acid (i.e. diet restriction). For example, an absence of a biomarker for a serine metabolism pathway in a subject can predict the subject having a positive response to a serine diet restriction therapy.

In some embodiments, a decreased level of a biomarker can predict a positive response to a nutrient modulation therapy. In some embodiments, a decreased level of a biomarker for an amino acid metabolism pathway in a subject, such as any of those described herein, can predict a positive response of the subject to a nutrient modulation therapy, wherein the nutrient modulation limits the amount of the amino acid (i.e. diet restriction). For example, a decreased level of a biomarker for a serine metabolism pathway in a subject can predict the subject having a positive response to a serine diet restriction therapy. In some embodiments, a decreased level of a biomarker can be identified when compared to a reference level. In some embodiments, the level of a biomarker can be decreased by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% compared to a reference level. In some embodiments, the level of a biomarker can be decreased from about 5% to about 15%, from about 10% to about 20%, from about 15% to about 25%, from about 20% to about 30%, from about 25% to about 35%, from about 30% to about 40%, from about 35% to about 45%, from about 40% to about 50%, from about 45% to about 55%, from about 50% to about 60%, from about 55% to about 65%, from about 60% to about 70%, from about 65% to about 75%, from about 70% to about 80%, from about 75% to about 85%, from about 80% to about 90%, from about 85% to about 95%, or from about 90% to about 100% compared to a reference level.

In some embodiments, a reference level of a biomarker can comprise the level of the biomarker in a normal or healthy cell or tissue or subject. In some embodiments, a normal or healthy cell, tissue, or subject may not comprise a disease or condition. In some embodiments, a normal or healthy cell, tissue, or subject may not have a cancer. In some embodiments, a normal or healthy subject may not have a higher risk of developing a cancer compared to an average subject in a population. In other embodiments, a reference level of a biomarker can comprise the level of the biomarker in a normal or healthy cell or tissue in a subject with a cancer.

In some embodiments, an absence or a decreased level of a biomarker for an amino acid metabolism pathway in a subject can be used to predict that the amino acid is essential or has become essential (i.e., conditionally essential) to the subject. In some embodiments, the essentiality of an amino acid can be specific to a group of subjects. In some embodiments, the essentiality of an amino acid can be specific to a subject.

In some embodiments, a positive response to a nutrient modulation therapy can reduce the size of a tumor. In some embodiments, a positive response to a nutrient modulation therapy can comprise a decrease in tumor size by at least about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to the tumor size before administering the nutrient modulation therapy. In some embodiments, a positive response to a nutrient modulation therapy can comprise a decrease in tumor size by about 1-10%, about 5-15%, about 10-20%, about 15-25%, about 20-30%, about 25-35%, about 30-40%, about 35-45%, about 40-50%, about 45-55%, about 50-60%, about 55-65%, about 60-70%, about 65-75%, about 70-80%, about 75-85%, about 80-90%, about 85-95%, or about 90-100% compared to the tumor size before administering the nutrient modulation therapy.

In some embodiments, a positive response to a nutrient modulation therapy can reduce the number of cancer cells. In some embodiments, a positive response to a nutrient modulation therapy can comprise a decrease in number of cancer cells by about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to the number of the cancer cells before administering the nutrient modulation therapy. In some embodiments, a positive response to a nutrient modulation therapy can comprise a decrease in number of cancer cells by at least about 1-10%, about 5-15%, about 10-20%, about 15-25%, about 20-30%, about 25-35%, about 30-40%, about 35-45%, about 40-50%, about 45-55%, about 50-60%, about 55-65%, about 60-70%, about 65-75%, about 70-80%, about 75-85%, about 80-90%, about 85-95%, or about 90-100% compared to the number of the cancer cells before administering the nutrient modulation therapy.

In some embodiments, a positive response to a nutrient modulation therapy can extend the life span of a subject. In some embodiments, a positive response to a nutrient modulation therapy can extend the life span of a subject by at least about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 1 month, about 2 months, about 3 months, about 4 months, about 5 months, about 6 months, about 7 months, about 8 months, about 9 months, about 10 months, about 11 months, about 12 months, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, about 31 years, about 32 years, about 33 years, about 34 years, about 35 years, about 36 years, about 37 years, about 38 years, about 39 years, about 40 years, about 41 years, about 42 years, about 43 years, about 44 years, about 45 years, about 46 years, about 47 years, about 48 years, about 49 years, about 50 years, or more than about 50 years. In some embodiments, a positive response to a nutrient modulation therapy can extend the life span of a subject by an amount of time from about 25 days to about 6 months, from about 5 months to about 12 months, from about 10 months to about 2 years, from about 1 year to about 5 years, from about 4 years to about 10 years, from about 9 years to about 15 years, from about 14 years to about 20 years, from about 19 years to about 25 years, from about 24 years to about 30 years, from about 29 years to about 35 years, from about 34 years to about 40 years, from about 39 years to about 45 years, or from about 44 years to about 50 years.

In some embodiments, a positive response to a nutrient modulation therapy can extend progression free survival of a subject. In some embodiments, a positive response to a nutrient modulation therapy can extend progression free survival of a subject by at least about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 1 month, about 2 months, about 3 months, about 4 months, about 5 months, about 6 months, about 7 months, about 8 months, about 9 months, about 10 months, about 11 months, about 12 months, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, about 31 years, about 32 years, about 33 years, about 34 years, about 35 years, about 36 years, about 37 years, about 38 years, about 39 years, about 40 years, about 41 years, about 42 years, about 43 years, about 44 years, about 45 years, about 46 years, about 47 years, about 48 years, about 49 years, about 50 years, or more than about 50 years. In some embodiments, a positive response to a nutrient modulation therapy can extend progression free survival of a subject by from about 14 days to about 6 months, from about 6 months to about 12 months, from about 12 months to about 2 years, from about 1 year to about 5 years, from about 5 years to about 10 years, from about 10 years to about 15 years, from about 15 years to about 20 years, from about 20 years to about 25 years, from about 25 years to about 30 years, from about 30 years to about 35 years, from about 35 years to about 40 years, from about 40 years to about 45 years, or from about 45 years to about 50 years. In some embodiments, a positive response to a nutrient modulation therapy can extend progression free survival of a subject by at least about 1 month. In some embodiments, a positive response to a nutrient modulation therapy can extend progression free survival of a subject by at least about 6 months.

In some embodiments, an absence or decreased level of at least one biomarker can predict a positive response to a nutrient modulation therapy. In some embodiments, an absence or decreased level of two biomarkers can predict a positive response to a nutrient modulation therapy. In some embodiments, an absence or decreased level of three biomarkers can predict a positive response to a nutrient modulation therapy. In some embodiments, an absence or decreased level of four biomarkers can predict a positive response to a nutrient modulation therapy. In some embodiments, an absence or decreased level of five biomarkers can predict a positive response to a nutrient modulation therapy. In some embodiments, an absence or decreased level of six biomarkers can predict a positive response to a nutrient modulation therapy. In some embodiments, an absence or decreased level of seven biomarkers can predict a positive response to a nutrient modulation therapy. In some embodiments, an absence or decreased level of eight biomarkers can predict a positive response to a nutrient modulation therapy. In some embodiments, an absence or decreased level of nine biomarkers can predict a positive response to a nutrient modulation therapy. In some embodiments, an absence or decreased level of ten biomarkers can predict a positive response to a nutrient modulation therapy. In some embodiments, an absence or decreased level of more than ten biomarkers can predict a positive response to a nutrient modulation therapy.

In some embodiments, a presence of a biomarker can predict a negative response to a nutrient modulation therapy. In some embodiments, a presence of a biomarker for an amino acid metabolism pathway in a subject, such as any of those described herein, can predict a negative response of the subject to a nutrient modulation therapy. For example, a presence of a biomarker for a serine metabolism pathway in a subject can predict the subject having a negative response to a serine modulation therapy.

In some embodiments, an increased level of a biomarker can predict a negative response to a nutrient modulation therapy. In some embodiments, an increased level of a biomarker for an amino acid metabolism pathway in a subject, such as any of those described herein, can predict a negative response of the subject to a nutrient modulation therapy. For example, an increased level of a biomarker for a serine metabolism pathway in a subject can predict the subject having a negative response to a serine modulation therapy. In some embodiments, an increased level can be identified when compared to a reference level. In some embodiments, an increased level of a biomarker can be a level that is at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, or about 500% higher than a reference biomarker level. In some embodiments, an increased level of a biomarker can be a level from about 5 to about 50%, from about 25 to about 75%, from about 50 to about 100%, from about 75 to about 200%, from about 150 to about 500% higher than a reference biomarker level.

In some embodiments, a method of the disclosure can comprise predicting a response of a subject to a nutrient modulation therapy based on a comparison of a plurality of biomarkers in a biological sample from a subject and a plurality of reference samples.

In some embodiments, a reference sample can comprise a normal or healthy cell or tissue of a subject. In some embodiments, a normal or healthy cell, tissue, or subject may not comprise a disease or a condition. In some embodiments, a normal or healthy cell, tissue, or subject may not have a cancer. In some embodiments, a normal or healthy subject may not have a higher risk of developing cancer when compared to an average subject in a population.

### Nutrient Modulation Therapy

In some embodiments, a nutrient modulation therapy changes the level of a metabolite or nutrient in a cell. In some embodiments, a nutrient modulation therapy increases the level of a metabolite or a nutrient in a cell. In some embodiments, a nutrient modulation therapy decreases the level of a metabolite or a nutrient in a cell. In some embodiments, a nutrient modulation therapy changes the level of a metabolite or nutrient in a cancer cell or tumor. In some embodiments, a nutrient modulation therapy changes the level of a metabolite or nutrient in systemic circulation or healthy tissues.

As described herein, the term “devoid of” refers to an amount of a substance in a composition. In some embodiments, a pharmaceutical composition or dietary product devoid of a nutrient or devoid of a component comprises less than about 5%, less than about 4.5%, less than about 4%, less than about 3.5%, less than about 3%, less than about 2.5%, less than about 2%, less than about 1.8%, less than about 1.6%, less than about 1.4%, less than about 1.2%, less than about 1%, less than about 0.8%, less than about 0.6%, less than about 0.5%, less than about 0.4%, less than about 0.3%, less than about 0.2%, or less than about 0.1% of the nutrient or component. In some embodiments, a pharmaceutical composition or dietary product devoid of a nutrient or devoid of a component comprises less than about 5% of the nutrient or component. In some embodiments, a pharmaceutical composition or dietary product devoid of a nutrient or devoid of a component comprises less than about 2.5% of the nutrient or component. In some embodiments, a pharmaceutical composition or dietary product devoid of a nutrient or devoid of a component comprises less than about 1% of the nutrient or component. In some embodiments, a pharmaceutical composition or dietary product devoid of a nutrient or devoid of a component comprises less than about 0.5% of the nutrient or component. In some embodiments, a pharmaceutical composition or dietary product devoid of a nutrient or devoid of a component comprises less than about 0.3% of the nutrient or component.

As described herein, modulating a nutrient level in a subject can comprise increasing or decreasing a nutrient level in a subject. In some embodiments, a nutrient level in a subject or a cell of a subject can be increased or decreased by modulating nutrient levels in the subject's daily dietary intake. In some embodiments, a nutrient level in a subject or a cell of a subject can be increased or decreased by modulating nutrient levels in the subject's meals. In some embodiments, a nutrient level in a subject or a cell of a subject can be increased or decreased by modulating nutrient levels in the subject's snacks.

In some embodiments, a nutrient level in a subject or a cell of a subject can be increased. In some embodiments, a nutrient level in a subject or a cell of a subject can be increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%. In some embodiments, a nutrient level in a subject or a cell of a subject can be increased by at least about 10%. In some embodiments, a nutrient level in a subject or a cell of a subject can be increased by at least about 20%. In some embodiments, a nutrient level in a subject or a cell of a subject can be increased by at least about 50%.

In some embodiments, a nutrient level in a subject can be increased in a subject by providing a meal or daily food intake comprising the nutrient in an amount greater than the daily recommended nutrient level for the subject. In some embodiments, a nutrient level in a subject can be increased in a subject by providing a meal or daily food intake comprising the nutrient in an amount that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% greater than the daily recommended nutrient level for the subject. In some embodiments, a nutrient level in a subject can be increased in a subject by providing a meal or daily food intake comprising the nutrient in an amount that is at least about 20% greater than the daily recommended nutrient level for the subject. In some embodiments, a nutrient level in a subject can be increased in a subject by providing a meal or daily food intake comprising the nutrient in an amount that is at least about 50% greater than the daily recommended nutrient level for the subject. In some embodiments, a nutrient level in a subject can be increased in a subject by providing a meal or daily food intake comprising the nutrient in an amount that is at least about 75% greater than the daily recommended nutrient level for the subject.

In some embodiments, a nutrient level in a subject or a cell of a subject can be decreased. In some embodiments, a nutrient level in a subject or a cell of a subject can be decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%. In some embodiments, a nutrient level in a subject or a cell of a subject can be decreased by at least about 10%. In some embodiments, a nutrient level in a subject or a cell of a subject can be decreased by at least about 20%. In some embodiments, a nutrient level in a subject or a cell of a subject can be decreased by at least about 50%.

In some embodiments, a nutrient level in a subject can be decreased in a subject by providing a meal or daily food intake comprising the nutrient in an amount less than the daily recommended nutrient level for the subject. In some embodiments, a nutrient level in a subject can be decreased in a subject by providing a meal or daily food intake comprising the nutrient in an amount that is at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, at most about 4.5%, at most about 4%, at most about 3.5%, at most about 3%, at most about 2.5%, at most about 2%, at most about 1.5%, at most about 1%, at most about 0.5%, or at most about 0.3% of the daily recommended nutrient level for the subject. In some embodiments, a nutrient level in a subject can be decreased in a subject by providing a meal or daily food intake comprising the nutrient in an amount that is at most about 75% of the daily recommended nutrient level for the subject. In some embodiments, a nutrient level in a subject can be decreased in a subject by providing a meal or daily food intake comprising the nutrient in an amount that is at most about 50% of the daily recommended nutrient level for the subject. In some embodiments, a nutrient level in a subject can be decreased in a subject by providing a meal or daily food intake comprising the nutrient in an amount that is at most about 20% of the daily recommended nutrient level for the subject. In some embodiments, a nutrient level in a subject can be decreased in a subject by providing a meal or daily food intake comprising the nutrient in an amount that is at most about 10% of the daily recommended nutrient level for the subject. In some embodiments, a nutrient level in a subject can be decreased in a subject by providing a meal or daily food intake comprising the nutrient in an amount that is at most about 5% of the daily recommended nutrient level for the subject. In some embodiments, a nutrient level in a subject can be decreased in a subject by providing a meal or daily food intake comprising the nutrient in an amount that is at most about 1% of the daily recommended nutrient level for the subject. In some embodiments, a nutrient level in a subject can be decreased in a subject by providing a meal or daily food intake comprising the nutrient in an amount that is at most about 0.5% of the daily recommended nutrient level for the subject.

In some embodiments, the nutrient modulated through meals or daily food intake is a protein. In some embodiments, protein levels can be modulated in a subject by administering to the subject meals with modified levels of protein or by modifying protein levels for the subject's daily food intake. In some embodiments, the nutrient modulated through meals or daily food intake is an amino acid. In some embodiments, one amino acid level can be modulated in a subject by administering to the subject meals with modified levels of the amino acid or by modifying amino acid levels for the subject's daily food intake. In some embodiments, two amino acid levels can be modulated in a subject by administering to the subject meals with modified levels of the amino acid or by modifying amino acid levels for the subject's daily food intake. In some embodiments, three amino acid levels can be modulated in a subject by administering to the subject meals with modified levels of the amino acid or by modifying amino acid levels for the subject's daily food intake. In some embodiments, the amino acid or combination of amino acids modulated by the subject's meals or daily food intake is an amino acid or amino acid combination of the disclosure, for example, serine, glycine, proline, tyrosine, cysteine, or a combination thereof. In some embodiments, the nutrient modulated through meals or daily food intake is a lipid. In some embodiments, the nutrient modulated through meals or daily food intake is a fatty acid.

In some embodiments, a nutrient modulation therapy can comprise a diet that has reduced levels of or is substantially devoid of at least one nutrient. In some embodiments, a nutrient modulation therapy can comprise a diet that is substantially devoid of at least one amino acid. In some embodiments, after detecting an absence or a decreased level of a biomarker of an amino acid metabolism pathway in a subject, the subject can be administered a nutrient modulation therapy that removes the amino acid from the subject's diet.

In some embodiments, a method of the disclosure can co-administer a nutrient modulation therapy and an agent that can deplete the abundance of a nutrient in a subject. In some embodiments, the agent is an enzyme that can degrade or deplete a nutrient in the subject. In some embodiments, after detecting an absence of a decreased level of a biomarker of an amino acid metabolism pathway in a subject, the subject can be administered with nutrient modulation therapy and an enzyme that can deplete the amino acid in the subject. In some embodiments, the agent can be an amino acid metabolism enzyme that depletes at least one metabolite associated with the amino acid metabolism pathway. In some embodiments, the agent is an enzyme. In some embodiments, the agent is an protein. In some embodiments, the agent is a small molecule inhibitor. In some embodiments, the agent is an antibody. In some embodiments, the agent is a metabolite.

In some embodiments, a nutrient modulation therapy can be administered by administering a dietary product. In some embodiments, a dietary product used for nutrient modulation therapy can be substantially devoid of or have a limited amount of histidine, arginine, alanine, isoleucine, cysteine, aspartic acid, leucine, glutamine, asparagine, lysine, glycine, glutamic acid, methionine, proline, serine, phenylalanine, tyrosine, threonine, tryptophan, or valine. In some embodiments, a dietary product can be substantially devoid of or have a limited amount of an essential amino acid, a conditionally essential amino acid, a nonessential amino acid, or any combination thereof. In some embodiments, a dietary product can be substantially devoid of or have a limited amount of histidine, isoleucine, leucine, lysine, methionine, phenylalanine, threonine, tryptophan, or valine. In some embodiments, a dietary product can be substantially devoid of or have a limited amount of arginine, cysteine, glutamine, glycine, proline, or tryptophan. In some embodiments, a dietary product can be substantially devoid of or have a limited amount of alanine, aspartic acid, asparagine, glutamic acid, or serine. In some embodiments, a dietary product can be substantially devoid of or have a limited amount of at least one of histidine, isoleucine, leucine, lysine, methionine, phenylalanine, threonine, tryptophan, valine, arginine, cysteine, glutamine, glycine, proline, tryptophan, alanine, aspartic acid, asparagine, glutamic acid, or serine. In some embodiments, a dietary product can be substantially devoid of or have a limited amount of at least two of histidine, isoleucine, leucine, lysine, methionine, phenylalanine, threonine, tryptophan, valine, arginine, cysteine, glutamine, glycine, proline, tryptophan, alanine, aspartic acid, asparagine, glutamic acid, or serine. In some embodiments, a dietary product can be substantially devoid of or have a limited amount of at least three, at least four, at least five or more of histidine, isoleucine, leucine, lysine, methionine, phenylalanine, threonine, tryptophan, valine, arginine, cysteine, glutamine, glycine, proline, tryptophan, alanine, aspartic acid, asparagine, glutamic acid, or serine.

In some embodiments, a dietary product can be substantially devoid of or have a limited amount of glycine. In some embodiments, a dietary product can be substantially devoid of or have a limited amount of serine. In some embodiments, a dietary product can be substantially devoid of or have a limited amount of proline. In some embodiments, a dietary product can be substantially devoid of or have a limited amount of cysteine. In some embodiments, a dietary product can be substantially devoid of or have a limited amount of tyrosine. In some embodiments, a dietary product can be substantially devoid of or have a limited amount of asparagine. In some embodiments, a dietary product can be substantially devoid of or have a limited amount of glutamine. In some embodiments, a dietary product can be substantially devoid of or have a limited amount of glutamate.

In some embodiments, a dietary product can be substantially devoid of at least one of histidine, isoleucine, leucine, lysine, methionine, phenylalanine, threonine, tryptophan, valine, arginine, cysteine, glutamine, glycine, proline, tryptophan, alanine, aspartic acid, asparagine, glutamic acid, or serine. In some embodiments, a dietary product can be substantially devoid of at least two of histidine, isoleucine, leucine, lysine, methionine, phenylalanine, threonine, tryptophan, valine, arginine, cysteine, glutamine, glycine, proline, tryptophan, alanine, aspartic acid, asparagine, glutamic acid, or serine. In some embodiments, a dietary product can be substantially devoid of at least three, at least four, at least five or more of histidine, isoleucine, leucine, lysine, methionine, phenylalanine, threonine, tryptophan, valine, arginine, cysteine, glutamine, glycine, proline, tryptophan, alanine, aspartic acid, asparagine, glutamic acid, or serine. In some embodiments, a dietary product can be substantially devoid of glycine. In some embodiments, a dietary product can be substantially devoid of serine. In some embodiments, a dietary product can be substantially devoid of proline.

In some embodiments, a dietary product can be substantially devoid of or have a limited amount of serine and glycine. In some embodiments, a dietary product can be substantially devoid of or have a limited amount of serine, glycine, and proline. In some embodiments, a dietary product can be substantially devoid of or have a limited amount of serine, glycine, and cysteine. In some embodiments, a dietary product can be substantially devoid of or have a limited amount of serine, glycine, and tyrosine. In some embodiments, a dietary product can be substantially devoid of or have a limited amount of serine, glycine, and asparagine. In some embodiments, a dietary product can be substantially devoid of or have a limited amount of serine, glycine, proline, and cysteine. In some embodiments, a dietary product can be substantially devoid of or have a limited amount of serine, glycine, proline, and tyrosine. In some embodiments, a dietary product can be substantially devoid of or have a limited amount of serine, glycine, proline, and asparagine. In some embodiments, a dietary product can be substantially devoid of or have a limited amount of serine, glycine, glutamate, glutamine, and cysteine.

A composition of the disclosure comprises at least ten amino acids or salts thereof. In some embodiments, a composition of the disclosure comprises 10, 11, 12, 13, 14, 15, 16, 17, 18, or 19 amino acids or a salt thereof. In some embodiments, a composition of the disclosure comprises 10 amino acids or a salt thereof. In some embodiments, a composition of the disclosure comprises 14 amino acids or a salt thereof. In some embodiments, a composition of the disclosure comprises 18 amino acids or a salt thereof. A salt of an amino acid disclosed herein can be a pharmaceutically acceptable salt. In some embodiments, a composition disclosed herein is devoid of serine and glycine. In some embodiments, a composition disclosed herein is devoid of serine. In some embodiments, a composition disclosed herein is devoid of glycine.

In some embodiments, a composition of the disclosure comprises 1, 2, 3, 4, 5, 6, 7, 8, or 9 essential amino acids or salts thereof. In some embodiments, a composition of the disclosure comprises 7, 8, or 9 essential amino acids or salts thereof. In some embodiments, a composition of the disclosure comprises 8 essential amino acids or salts thereof. In some embodiments, a composition of the disclosure comprises 9 essential amino acids or salts thereof. A salt of an amino acid disclosed herein can be a pharmaceutically acceptable salt.

In some embodiments, a composition of the disclosure comprises 1, 2, 3, 4, 5, 6, 7, 8, 9, 10, or 11 non-essential amino acids or salts thereof. In some embodiments, a composition of the disclosure comprises 7, 8, 9, 10, or 11 non-essential amino acids or salts thereof. In some embodiments, a composition of the disclosure comprises 7 non-essential amino acids or salts thereof. In some embodiments, a composition of the disclosure comprises 8 non-essential amino acids or salts thereof. In some embodiments, a composition of the disclosure comprises 9 non-essential amino acids or salts thereof. A salt of an amino acid disclosed herein can be a pharmaceutically acceptable salt. In some embodiments, a composition disclosed herein is devoid of serine and glycine. In some embodiments, a composition disclosed herein is devoid of proline, serine, and glycine. In some embodiments, a composition disclosed herein is devoid of serine. In some embodiments, a composition disclosed herein is devoid of a combination of more than 1 of proline, serine, or glycine. In some embodiments, a composition disclosed herein is devoid of proline. In some embodiments, a composition disclosed herein is devoid of serine. In some embodiments, a composition disclosed herein is devoid of glycine.

A composition of the disclosure can comprise essential amino acids or salts thereof and non-essential amino acids or salts thereof. In some embodiments, a composition of the disclosure comprises 1, 2, 3, 4, 5, 6, 7, 8, or 9 essential amino acids or salts thereof and 1, 2, 3, 4, 5, 6, 7, 8, 9, 10, or 11 non-essential amino acids or salts thereof. In some embodiments, a composition of the disclosure comprises 7, 8, or 9 essential amino acids or salts thereof and 6, 7, 8, or 9 non-essential amino acids or salts thereof. In some embodiments, a composition of the disclosure comprises 8 or 9 essential amino acids or salts thereof and 8 or 9 non-essential amino acids or salts thereof. In some embodiments, a composition of the disclosure comprises 9 essential amino acids or salts thereof and 7 non-essential amino acids or salts thereof. In some embodiments, a composition of the disclosure comprises 9 essential amino acids or salts thereof and 8 non-essential amino acids or salts thereof. In some embodiments, a composition of the disclosure comprises 9 essential amino acids or salts thereof and 9 non-essential amino acids or salts thereof. A salt of an amino acid disclosed herein can be a pharmaceutically acceptable salt. In some embodiments, a composition disclosed herein is devoid of serine and glycine. In some embodiments, a composition disclosed herein is devoid of proline, serine, and glycine. In some embodiments, a composition disclosed herein is devoid of serine. In some embodiments, a composition disclosed herein is devoid of a combination of more than 1 of proline, serine, or glycine. In some embodiments, a composition disclosed herein is devoid of proline. In some embodiments, a composition disclosed herein is devoid of serine. In some embodiments, a composition disclosed herein is devoid of glycine.

In some embodiments, a composition of the disclosure comprises histidine, isoleucine, leucine, lysine, methionine, cysteine, phenylalanine, tyrosine, threonine, tryptophan, valine, arginine, glutamine, alanine, aspartic acid, asparagine, glutamic acid or proline. In some embodiments, a composition of the disclosure comprises L-histidine, L-isoleucine, L-leucine, L-lysine, L-methionine, L-cysteine, L-phenylalanine, L-tyrosine, L-threonine, L-tryptophan, L-valine, L-arginine, L-glutamine, L-alanine, L-aspartic acid, L-asparagine, L-glutamic acid, or L-proline.

In some embodiments, a composition comprises histidine or a salt thereof, such as L-histidine or L-histidine hydrochloride. In some embodiments, a composition of the disclosure comprises isoleucine or a salt thereof, such as L-isoleucine, L-isoleucine methyl ester hydrochloride, or L-isoleucine ethyl ester hydrochloride. A salt of an amino acid disclosed herein can be a pharmaceutically acceptable salt. In some embodiments, a composition of the disclosure comprises leucine or a salt thereof, such as L-leucine, L-leucine methyl ester hydrochloride, or L-leucine ethyl ester hydrochloride. A salt of an amino acid disclosed herein can be a pharmaceutically acceptable salt. In some embodiments, a composition of the disclosure comprises lysine or a salt thereof, such as L-lysine, L-lysine hydrochloride, or L-lysine dihydrochloride. A salt of an amino acid disclosed herein can be a pharmaceutically acceptable salt. In some embodiments, a composition of the disclosure comprises methionine or a salt thereof, such as L-methionine, L-methionine methyl ester hydrochloride, or L-methionine hydrochloride. A salt of an amino acid disclosed herein can be a pharmaceutically acceptable salt.

In some embodiments, a composition of the disclosure comprises cysteine or a salt thereof, such as L-cysteine, L-cysteine hydrochloride, L-cysteine methyl ester hydrochloride, or L-cysteine ethyl ester hydrochloride. In some embodiments, a composition discloses cystine or a salt thereof, such as L-cystine. A salt of an amino acid disclosed herein can be a pharmaceutically acceptable salt. In some embodiments, a composition of the disclosure comprises phenylalanine or a salt thereof, such as L-phenylalanine, DL-phenylalanine, or L-phenylalanine methyl ester hydrochloride. In some embodiments, a composition of the disclosure comprises tyrosine or a salt thereof, such as L-tyrosine or L-tyrosine hydrochloride. In some embodiments, a composition of the disclosure comprises threonine or a salt thereof, such as L-threonine or L-threonine methyl ester hydrochloride. In some embodiments, a composition of the disclosure comprises L-tryptophan. In some embodiments, a composition of the disclosure comprises valine or a salt thereof, such as L-valine, L-valine methyl ester hydrochloride, or L-valine ethyl ester hydrochloride. A salt of an amino acid disclosed herein can be a pharmaceutically acceptable salt.

In some embodiments, a composition of the disclosure comprises arginine or a salt thereof, such as L-arginine or L-arginine hydrochloride. In some embodiments, a composition of the disclosure comprises glutamine or a salt thereof, such as L-glutamine or L-glutamine hydrochloride. In some embodiments, a composition of the disclosure comprises alanine or a salt thereof, such as L-alanine or β-alanine. In some embodiments, a composition of the disclosure comprises aspartic acid or a salt thereof, such as L-aspartic acid, D-aspartic acid, L- or D-aspartic acid potassium salt, L- or D-aspartic acid hydrochloride salt; L- or D-aspartic acid magnesium salt, or L- or D-aspartic acid calcium salt. In some embodiments, a composition of the disclosure comprises L-asparagine. In some embodiments, a composition of the disclosure comprises glutamic acid or a salt thereof, such as L-glutamic acid or L-glutamic acid hydrochloride. In some embodiments, a composition of the disclosure comprises proline or a salt thereof, such as L-proline, L-proline hydrochloride, L-proline methyl ester hydrochloride, or L-proline ethyl ester hydrochloride. A salt of an amino acid disclosed herein can be a pharmaceutically acceptable salt.

In some embodiments, a composition of the disclosure can be devoid of at least 1, 2, 3, 4, 5, 6, 7, or 8 amino acids. In some embodiments, a composition of the disclosure can be devoid of at least 1 acid. In some embodiments, a composition of the disclosure can be devoid of at least 2 acids. In some embodiments, a composition of the disclosure can be devoid of at least 3 acids. In some embodiments, a composition of the disclosure can be devoid of at least 4 acids. In some embodiments, a composition of the disclosure can be devoid of at least 5 acids.

In some embodiments, a composition of the disclosure can be devoid of at least serine. In some embodiments, a composition of the disclosure can be devoid of at least glycine. In some embodiments, a composition of the disclosure can be devoid of at least proline. In some embodiments, a composition of the disclosure can be devoid of at least serine and glycine. In some embodiments, a composition of the disclosure can be devoid of at least serine, glycine, and proline.

### Pharmaceutical Compositions

A pharmaceutical composition of the disclosure can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

A pharmaceutical composition of the disclosure can be a combination of any compositions or dietary products described herein with other chemical components, such as carriers, stabilizers, diluents, dispersing agents, suspending agents, thickening agents, and/or excipients. The pharmaceutical composition facilitates administration of the dietary product to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration. In some embodiments, the composition or dietary product of the disclosure is administered orally.

A pharmaceutical composition can be administered in a local manner, for example, via injection of the dietary product directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compositions or dietary products of the disclosure with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino) propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremophor, DMSO, and potassium phosphate buffer.

Pharmaceutical compositions can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical compositions for parenteral administration include aqueous solutions of the active dietary products in water-soluble form. Suspensions of the active dietary products can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the dietary products to allow for the preparation of highly concentrated solutions. Alternatively, the active ingredient can be in powder form for constitution with a suitable vehicle, e.g., sterile pyrogen-free water, before use.

In practicing a method of treatment or use provided herein, therapeutically-effective amounts of the dietary products described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the dietary products used, and other factors. The dietary products can be used singly or in combination with one or more therapeutic agents as components of mixtures.

Pharmaceutical compositions can be formulated using one or more physiologically-acceptable carriers comprising excipients and auxiliaries, which facilitate processing of the active dietary products into preparations that can be used pharmaceutically. Formulations can be modified depending upon the route of administration chosen. Pharmaceutical compositions comprising a dietary product described herein can be manufactured, for example, by mixing, dissolving, emulsifying, encapsulating, entrapping, or compression processes.

The pharmaceutical compositions can include at least one pharmaceutically-acceptable carrier, diluent, or excipient and dietary products described herein as free-base or pharmaceutically-acceptable salt form. Pharmaceutical compositions can contain solubilizers, stabilizers, tonicity enhancing agents, buffers and preservatives.

Methods for the preparation of compositions comprising the dietary products described herein include formulating the dietary products with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a dietary product is dissolved, emulsions comprising a dietary product, or a solution containing liposomes, micelles, or nanoparticles comprising a dietary product as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

Anti-adherent: A composition of the disclosure can comprise an anti-adherent. In some embodiments, a composition of the disclosure can comprise an anti-adherent, such as magnesium stearate.

Binding agent: A composition of the disclosure can comprise at least one binding agent to hold the composition together. In some embodiments, a composition of the disclosure can comprise a binding agent, such as a saccharide, protein, or synthetic polymer. In some embodiments, a composition of the disclosure can comprise a disaccharide (e.g., sucrose or lactose), a polysaccharide or polysaccharide derivative (e.g., starch, cellulose, modified cellulose, cellulose ether), or a sugar alcohol (e.g., xylitol, sorbitol, or mannitol). In some embodiments, a composition of the disclosure can comprise a protein binder, such as gelatin. In some embodiments, a composition of the disclosure can comprise a synthetic polymer binder, such as polyvinylpyrrolidone (PVP) or polyethylene glycol (PEG).

Preservative: A composition of the disclosure can comprise at least one preservative. In some embodiments, a composition of the disclosure can comprise an antioxidant or an antimicrobial. Antioxidant agents delay or prevent the deterioration of the composition by oxidative mechanisms. Antimicrobial agents inhibit the growth of spoilage or pathogenic microorganisms in the composition.

Antioxidant: In some embodiments, an antioxidant agent is added to the composition to delay or prevent autooxidation of unsaturated fatty acids or enzyme-catalyzed oxidation. In some embodiments, a composition of the disclosure comprises at least one of ascorbic acid, butylated hydroxyanisole (BHA), butylated hydroxytoluene (BHT), citric acid, a sulfite, tertiary butylhydroquinone (TBHQ), or a tocopherol. In some embodiments, a composition of the disclosure comprises ascorbic acid. In some embodiments, a composition of the disclosure comprises BHT. In some embodiments, a composition of the disclosure comprises citric acid.

In some embodiments, a composition of the disclosure comprises about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg of an antioxidant agent. In some embodiments, a composition of the disclosure comprises up to about 100 mg/kg, up to about 200 mg/kg, up to about 300 mg/kg, up to about 400 mg/kg, up to about 500 mg/kg, up to about 600 mg/kg, up to about 700 mg/kg, up to about 800 mg/kg, up to about 900 mg/kg, or up to about 1000 mg/kg of an antioxidant agent.

Antimicrobial agent: In some embodiments, an antimicrobial agent is added to the composition to delay or prevent growth of spoilage or pathogenic microorganisms in the composition. In some embodiments, a composition of the disclosure comprises at least one of acetic acid, benzoic acid, natamycin, nisin, a nitrate, a nitrite, propionic acid, sorbic acid, a sulfite, or sulfur dioxide.

In some embodiments, a composition of the disclosure comprises about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg of an antimicrobial agent. In some embodiments, a composition of the disclosure comprises up to about 100 mg/kg, up to about 200 mg/kg, up to about 300 mg/kg, up to about 400 mg/kg, up to about 500 mg/kg, up to about 600 mg/kg, up to about 700 mg/kg, up to about 800 mg/kg, up to about 900 mg/kg, or up to about 1000 mg/kg of an antimicrobial agent.

Colorants: A composition of the disclosure can comprise at least one colorant. In some embodiments, a composition of the disclosure comprises a natural colorant or a synthetic colorant. In some embodiments, a composition of the disclosure comprises a natural colorant. In some embodiments, a composition of the disclosure comprises an anthocyanin. In some embodiments, a composition of the disclosure comprises an anthocyanin, such as pelargonidin-3-glucoside obtained from strawberries (Fragaria species) or malvidin-3-glucoside obtained from grapes (Vitis species). In some embodiments, a composition of the disclosure comprises a betacyanin. In some embodiments, a composition of the disclosure comprises a betacyanin, such as betanin obtained from beet root (Beta vulgaris). In some embodiments, a composition of the disclosure comprises a carotenoid. In some embodiments, a composition of the disclosure comprises a carotenoid, such as bixin obtained from annatto (Bixa Orellana); crocin obtained from saffron (Crocus sativus); capsanthin obtained from paprika (Capsicum annuum); beta-carotene obtained from carrot (Daucus carota); or canthaxanthin obtained from mushrooms (Cantharellus cinnabarinus). In some embodiments, a composition of the disclosure comprises a phenolic. In some embodiments, a composition of the disclosure comprises a phenolic, such as curcumin obtained from turmeric (Cuycuma longa).

In some embodiments, a composition of the disclosure comprises a synthetic colorant. In some embodiments, a composition of the disclosure comprises allura red AC, brilliant blue FCF, erythrosine, fast green FCF, indico carmine, sunset yellow FCF, or tartrazine. In some embodiments, a composition of the disclosure comprises FD&C red no. 40, FD&C blue no. 1, FD&C red no. 3, FD&C green no. 3, FD&C blue no. 2, FD&C yellow no. 6, or FD&C yellow no. 5. In some embodiments, a composition of the disclosure comprises E133, E127, E132, E110, or E102.

Flavorants: A composition of the disclosure can comprise at least one flavoring agent. In some embodiments, a composition of the disclosure can comprise a natural flavoring substance, a nature-identical flavoring substance, or an artificial flavoring substance. In some embodiments, a composition of the disclosure can comprise a natural flavoring substance, such as a spice, fruit juice, or vegetable juice. In some embodiments, a composition of the disclosure can comprise a nature-identical flavoring substance, such as vanillin.

In some embodiments, a composition of the disclosure can comprise an artificial flavoring substance, such as allylpyrazine, methoxypyrazine, 2-iso-butyl-3-methoxypyrazine, acetyl-L-pyrazine, 2-acetoxy pyrazine, aldehydes, alcohols, esters, ketones, pyrazines, phenolics, or terpenoids.

Sweetener: A composition of the disclosure can comprise at least one sweetener. In some embodiments, a composition of the disclosure comprises sucrose, glucose, fructose, corn syrup, high-fructose corn syrup, or a sugar alcohol. In some embodiments, a composition of the disclosure comprises a sugar alcohol, such as sorbitol, mannitol, or xylitol. In some embodiments, a composition of the disclosure comprises fructose. In some embodiments, a composition of the disclosure comprises a synthetic sweetener. In some embodiments, a composition of the disclosure comprises saccharin, a cyclamate, aspartame, or acesulfame K. In some embodiments, a composition of the disclosure comprises aspartame.

In some embodiments, a composition of the disclosure can comprise a sweetener in an amount of about 0.5 g/serving, about 1 g/serving, about 1.5 g/serving, about 2 g/serving, about 2.5 g/serving, about 3 g/serving, about 3.5 g/serving, about 4 g/serving, about 4.5 g/serving, about 5 g/serving, about 5.5 g/serving, about 6 g/serving, about 6.5 g/serving, about 7 g/serving, about 7.5 g/serving, about 8 g/serving, about 8.5 g/serving, about 9 g/serving, about 9.5 g/serving, or about 10 g/serving. In some embodiments, a composition of the disclosure can comprise about 1 g/serving of a sweetener. In some embodiments, a composition of the disclosure can comprise about 2.5 g/serving of a sweetener. In some embodiments, a composition of the disclosure can comprise about 5 g/serving of a sweetener.

Processing agents: A composition of the disclosure can comprise at least one processing additive. In some embodiments, a composition of the disclosure can comprise an anticaking agent, a bleaching agent, a chelating agent, a clarifying agent, conditioning agent, emulsifying agent, a humectant, a pH control agent, a stabilizing agent, or a thickening agent. In some embodiments, a composition of the disclosure can comprise an anticaking agent such as sodium aluminosilicate, a chelating agent such as ethylenediaminetetraacetic acid (EDTA), a conditioning agent such as potassium bromate, or a pH control agent such as citric acid or lactic acid. In some embodiments, a composition of the disclosure can comprise a humectant such as glycerol, or a stabilizing and thickening agent such as pectin, gelatin, carrageenan, or guar gum.

Other agents: In some embodiments, the composition further comprises from about 0.5% to about 5% of a K⁺ source. In some embodiments, the composition further comprises at least about 0.5%, at least about 1%, at least about 1.5%, at least about 2%, at least about 2.5%, at least about 3%, at least about 3.5%, at least about 4%, at least about 4.5%, or at least about 5% of a K⁺ source. In some embodiments, the composition further comprises at least about 1% of a K⁺ source. In some embodiments, the composition further comprises at least about 2% of a K⁺ source. In some embodiments, the composition further comprises about 0.5%, about 1%, about 1.5%, about 2%, about 2.5%, about 3%, about 3.5%, about 4%, about 4.5%, or about 5% of a K⁺ source. In some embodiments, the composition further comprises about 2% of a K⁺ source. In some embodiments, the composition further comprises about 5% of a K⁺ source. In some embodiments, the K⁺ source comprises potassium citrate, potassium phosphate, potassium chloride, potassium sulfate, potassium gluconate, potassium bicarbonate, potassium aspartate, potassium acetate, and potassium orotate.

In some embodiments, the composition further comprises from about 0.5% to about 5% (w/w) of a Mg²⁺ source. In some embodiments, the composition further comprises at least about 0.5%, at least about 1%, at least about 1.5%, at least about 2%, at least about 2.5%, at least about 3%, at least about 3.5%, at least about 4%, at least about 4.5%, or at least about 5% of a Mg²⁺ source. In some embodiments, the composition further comprises at least about 1% of a Mg²⁺ source. In some embodiments, the composition further comprises at least about 2% of a Mg²⁺ source. In some embodiments, the composition further comprises about 0.5%, about 1%, about 1.5%, about 2%, about 2.5%, about 3%, about 3.5%, about 4%, about 4.5%, or about 5% of a Mg²⁺ source. In some embodiments, the composition further comprises about 2% of a Mg²⁺ source. In some embodiments, the composition further comprises about 4% of a Mg²⁺ source. In some embodiments, the Mg²⁺ source comprises magnesium citrate, magnesium fumarate, magnesium acetate, magnesium aspartate, magnesium threonate, magnesium glycinate, magnesium chloride, magnesium sulfate, magnesium oxide, or magnesium malate, magnesium orotate, or a hydrate thereof.

In some embodiments, the composition further comprises taurine, a hydrate thereof, or a salt thereof in an amount of from about 0.05% to about 2% (w/w). In some embodiments, the composition further comprises taurine, a hydrate thereof, or a salt thereof in an amount of at least about 0.05%, at least about 1%, at least about 1.2%, at least about 1.4%, at least about 1.6%, at least about 1.8%, or at least about 2%. In some embodiments, the composition further comprises taurine, a hydrate thereof, or a salt thereof in an amount of at least about 0.05%. In some embodiments, the composition further comprises taurine, a hydrate thereof, or a salt thereof in an amount of at least about 1%. In some embodiments, the composition further comprises taurine, a hydrate thereof, or a salt thereof in an amount of at least about 2%.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania, 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), each of which is incorporated by reference in its entirety.

A composition of the disclosure can be, for example, an immediate release form or a controlled release formulation. An immediate release formulation can be formulated to allow the dietary products to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a dietary product's action for an extended period of time. A delayed release form can be formulated to delay the release of an effective dose of one or more dietary products, for example, for about 4, about 8, about 12, about 16, or about 24 h.

A controlled release formulation can be a sustained release form. A sustained release form can be formulated to sustain, for example, the dietary product's action over an extended period of time. A sustained release form can be formulated to provide an effective dose of any dietary product described herein (e.g., provide a physiologically-effective blood profile) over about 4, about 8, about 12, about 16, or about 24 h.

Pharmaceutical compositions described herein can be in unit dosage forms suitable for single administration of precise dosages. In unit dosage form, the formulation is divided into unit doses containing appropriate quantities of one or more dietary products. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material, and spheronization agents, and any combination thereof.

A composition of the disclosure can comprise at least one pharmaceutical excipient, such as an anti-adherent, a binder, coating, colorant, disintegrant, flavorant, preservative, sorbent, sweetener, or vehicle. In some embodiment, a composition of the disclosure comprises a colorant and a flavorant. In some embodiment, a composition of the disclosure comprises a colorant, flavorant, and sweetener. In some embodiment, a composition of the disclosure comprises a flavorant, sweetener, and a preservative.

Supplements: A composition of the disclosure can be administered to a subject that is administered at least one supplement. In some embodiments, a composition of the disclosure is administered to the subject with an energy supplement. In some embodiments, a composition of the disclosure is administered to the subject with an energy supplement, such as caffeine, guarana, Asian ginseng, vitamin B12, or coenzyme Q10. In some embodiments, a composition of the disclosure is administered to the subject with an energy supplement, such as caffeine, tyrosine, pyrroloquinoline quinone (PQQ), theanine, coenzyme Q10, acetyl-L-carnitine (ALCAR), alpha-lipoic acid (ALA), citicoline, creatine, citrulline, beetroot powder, Ashwagandha, or Rhoodiola rosea. In some embodiments, a composition of the disclosure is administered to the subject with coenzyme Q10.

In some embodiments, a composition of the disclosure is administered to the subject with a micronutrient supplement. In some embodiments, a composition of the disclosure is administered to the subject with a multivitamin. In some embodiments, a composition of the disclosure is administered to the subject with a vitamin supplement, such as vitamin C or vitamin D supplement. In some embodiments, a composition of the disclosure is administered to the subject with a mineral supplement, such as an iron or zinc supplement.

In some embodiments, a composition of the disclosure is administered to the subject with an energy supplement and a micronutrient supplement. In some embodiments, a composition of the disclosure is administered to a subject with coenzyme Q10 and a multivitamin. In some embodiments, a composition of the disclosure is administered to a subject with coenzyme Q10 and a mineral supplement.

Vitamins: A composition of the disclosure can comprise one or more essential vitamins. In some embodiments, a composition of the disclosure can comprise vitamin A, vitamin C, vitamin D, vitamin E, vitamin K, and vitamin B. In some embodiments, a composition of the disclosure can comprise thiamine (vitamin B1), riboflavin (vitamin B2), niacin (vitamin B3), pantothenic acid (vitamin B5), pyroxidine (vitamin B6), biotin (vitamin B7), folate (vitamin B9), or cobalamin (vitamin B12). In some embodiments, a composition of the disclosure can comprise a fat-soluble vitamin, such as vitamin A, vitamin D, vitamin E, or vitamin K. In some embodiments, a composition of the disclosure can comprise a water-soluble vitamin, such as vitamin C and vitamin B. In some embodiments, a composition of the disclosure can comprise a water-soluble vitamin, such as vitamin B, such as thiamine (vitamin B1), riboflavin (vitamin B2), niacin (vitamin B3), pantothenic acid (vitamin B5), pyroxidine (vitamin B6), biotin (vitamin B7), folate (vitamin B9), or cobalamin (vitamin B12).

In some embodiments, a composition of the disclosure can comprise vitamin A, vitamin B, vitamin C, vitamin D, and vitamin E. In some embodiments, a composition of the disclosure can comprise vitamin A, vitamin C, vitamin D, vitamin E, thiamine (vitamin B1), riboflavin (vitamin B2), niacin (vitamin B3), pantothenic acid (vitamin B5), pyroxidine (vitamin B6), biotin (vitamin B7), folate (vitamin B9), and cobalamin (vitamin B12).

A composition of the disclosure can comprise the recommended dietary allowance of vitamins in a male adult. In some embodiments, a composition of the disclosure comprises the recommended dietary allowance of vitamins in a male adult: vitamin A, 900 μg; vitamin C, 90 mg; vitamin D, 15 μg; vitamin E, 15 mg; vitamin K, 120 μg; thiamine, 1.2 mg; riboflavin, 1.3 mg; niacin, 16 mg; pantothenic acid, 5 mg; pyroxidine, 1.3 mg; biotin, 30 μg; folate, 400 μg; and choline, 550 mg. A composition of the disclosure can comprise the recommended dietary allowance of vitamins in a female adult. In some embodiments, a composition of the disclosure comprises the recommended dietary allowance of vitamins in a female adult: vitamin A, 700 μg; vitamin C, 75 mg; vitamin D, 15 μg; vitamin E, 15 mg; vitamin K, 90 μg; thiamine, 1.1 mg; riboflavin, 1.1 mg; niacin, 14 mg; pantothenic acid, 5 mg; pyroxidine, 1.3 mg; biotin, 30 μg; folate, 400 μg; and choline, 425 mg.

A composition of the disclosure can comprise about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of any of the recommended dietary allowance of vitamins in a male or female adult. In some embodiments, a composition of the disclosure can comprise about 20%, about 30%, about 40%, about 50%, or about 60% of any of the recommended dietary allowance of vitamins in a male or female adult. In some embodiments, a composition of the disclosure can comprise about 30% of any of the recommended dietary allowance of vitamins in a male or female adult. In some embodiments, a composition of the disclosure can comprise about 50% of any of the recommended dietary allowance of vitamins in a male or female adult.

Minerals: A composition of the disclosure can comprise one or more minerals or elements. In some embodiments, a composition of the disclosure can comprise calcium, chromium, copper, fluoride, iodide, iron, magnesium, manganese, molybdenum, phosphorous, selenium, zinc, potassium, sodium, or chloride.

A composition of the disclosure can comprise the recommended daily allowance of elements or minerals in a male adult. In some embodiments, a composition of the disclosure comprises a recommended daily allowance of elements or minerals in a male adult: calcium, 1000 mg; chromium, 35 μg; copper, 900 μg; fluoride, 4 mg; iodide, 150 μg; iron, 8 mg; magnesium, 400 mg; manganese, 2.3 mg; molybdenum, 45 μg; phosphorous, 700 mg; selenium, 55 μg; zinc, 11 mg; potassium, 3400 mg; sodium, 1500 mg; or chloride, 2.3 g. A composition of the disclosure can comprise the recommended daily allowance of elements or minerals in a female adult. In some embodiments, a composition of the disclosure comprises a recommended daily allowance of elements or minerals in a female adult: calcium, 1000 mg; chromium, 25 μg; copper, 900 μg; fluoride, 3 mg; iodide, 150 μg; iron, 18 mg; magnesium, 310 mg; manganese, 1.8 mg; molybdenum, 45 μg; phosphorous, 700 mg; selenium, 55 μg; zinc, 8 mg; potassium, 2600 mg; sodium, 1500 mg; or chloride, 2.3 g.

A composition of the disclosure can comprise about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of any of the recommended dietary allowance of elements or minerals in a male or female adult. In some embodiments, a composition of the disclosure can comprise about 20%, about 30%, about 40%, about 50%, or about 60% of any of the recommended dietary allowance of elements or minerals in a male or female adult. In some embodiments, a composition of the disclosure can comprise about 30% of any of the recommended dietary allowance of elements or minerals in a male or female adult. In some embodiments, a composition of the disclosure can comprise about 50% of any of the recommended dietary allowance of elements or minerals in a male or female adult.

Lipids: A composition of the disclosure can comprise fat. In some embodiments, a pharmaceutical composition of the disclosure can comprise saturated fat, trans fat, polyunsaturated fat, or monounsaturated fat. In some embodiments, a composition of the disclosure can comprise saturated fat, trans fat, polyunsaturated fat, and monounsaturated fat. In some embodiments, a composition of the disclosure can comprise about 1 g, about 2 g, about 3 g, about 4 g, about 5 g, about 6 g, about 7 g, about 8 g, about 9 g, or about 10 g of combined fat content. In some embodiments, a composition of the disclosure can comprise about 5 g of combined fat content.

In some embodiments, a composition of the disclosure comprises cholesterol. In some embodiments, a composition of the disclosure comprises about 100 mg/serving of cholesterol. In some embodiments, a composition of the disclosure comprises about 50 mg/serving of cholesterol.

Carbohydrates: In some embodiments, a composition of the disclosure comprises a carbohydrate, such as a sugar, starch, or complex carbohydrate. In some embodiments, a composition of the disclosure comprises a sugar, such as corn syrup, fructose, galactose, glucose, high fructose corn syrup, lactose, maltose, or sucrose. In some embodiments, a composition of the disclosure comprises a sugar alcohol. In some embodiments, a composition of the disclosure comprises a starch. In some embodiments, a composition of the disclosure comprises a resistant starch, such as oats, rice, legumes, raw potato starch, green bananas, or Hi-Maize® flour.

In some embodiments, a composition of the disclosure comprises a complex carbohydrate, such as fiber. In some embodiments, a composition of the disclosure comprises a soluble fiber. In some embodiments, a composition of the disclosure comprises soluble fiber obtained from a food source, such as oatmeal, flax seed, barley, dried peas, apples, or carrots. In some embodiments, a composition of the disclosure comprises a insoluble fiber. In some embodiments, a composition of the disclosure comprises insoluble fiber obtained from a food source, such as seeds, nuts, dark green leafy vegetables, or wheat bran. In some embodiments, a composition of the disclosure comprises fiber, such as inulin, methylcellulose, psyllium, or wheat dextrin.

In some embodiments, a composition of the disclosure is administered to a subject as a nutritionally complete product. In some embodiments, the composition is administered as a meal replacement shake or powder. In some embodiments, the composition is administered via an enteral feeding tube. In some embodiments, the composition is administered as an intravenous topiramate.

### Dosing

Pharmaceutical compositions described herein can be in unit dosage forms suitable for single administration of precise dosages. In unit dosage form, the formulation is divided into unit doses containing appropriate quantities of one or more dietary products. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A dose can be expressed in terms of an amount of the drug divided by the mass of the subject, for example, milligrams of drug per kilograms of subject body mass.

A composition described herein can be given to supplement a meal consumed by a subject. A composition described herein can be given as a meal replacement. A composition described herein can be given immediately before or immediately after a meal. A composition described here can be given within about 5 minutes, about 10 minutes, about 15 minutes, about 20 minutes, about 25 minutes, about 30 minutes, about 40 minutes, about one hour, about 2 hours, about 3 hours, about 4 hours, about 5 hours, or about 6 hours before or after a meal.

A composition described herein can be in unit dosage forms suitable for single administration of precise dosages. In unit dosage form, the formulation is divided into unit doses containing appropriate quantities of the composition. In some embodiments, the unit dosage can be in the form of a package containing discrete quantities of the formulation. In some embodiments, formulations of the disclosure can be presented in unit dosage form in a single-serving sachet. For example, a single-serving sachet is dissolved in water or other suitable excipient or vehicle. In some embodiments, formulations of the disclosure can be presented in a single-dose non-reclosable container. In some embodiments, a formulation of the disclosure can be presented in a reclosable container, and the subject can obtain a single-dose serving of the formulation using a scoop or spoon designed to distribute a single-dose serving. In some embodiments, a formulation of the disclosure can be presented in a reclosable container, and the subject can obtain a single-dose serving of the formulation using a scoop or spoon designed to distribute a half-dose serving (i.e., two scoops to distribute one serving).

A composition described herein can be present in a unit dose serving in a range from about 1 g to about 2 g, from about 2 g to about 3 g, from about 3 g to about 4 g, from about 4 g to about 5 g, from about 5 g to about 6 g, from about 6 g to about 7 g, from about 7 g to about 8 g, from about 8 g to about 9 g, from about 9 g to about 10 g, from about 10 g to about 11 g, from about 11 g to about 12 g, from about 12 g to about 13 g, from about 13 g to about 14 g, from about 14 g to about 15 g, from about 15 g to about 16 g, from about 16 g to about 17 g, from about 17 g to about 18 g, from about 18 g to about 19 g, from about 19 g to about 20 g, from about 20 g to about 21 g, from about 21 g to about 22 g, from about 22 g to about 23 g, from about 23 g to about 24 g, or from about 24 g to about 25 g.

A composition described herein can be present in a unit dose serving in an amount of about 1 g, about 2 g, about 3 g, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 19 g, about 20 g, about 21 g, about 22 g, about 23 g, about 24 g, or about 25 g. In some embodiments, a composition described herein is present in a unit dose serving in an amount of about 10 g, 12 g, 15 g, 20 g, or 24 g.

In some embodiments, a composition described herein is present in a unit dose serving in an amount of about 12 g. In some embodiments, a composition described herein is present in a unit dose serving in a sachet in an amount of about 12 g. In some embodiments, a composition described herein is present in a unit dose serving in an amount of about 15 g. In some embodiments, a composition described herein is present in a unit dose serving in a sachet in an amount of about 15 g. In some embodiments, a composition described herein is present in a unit dose serving in an amount of about 24 g. In some embodiments, a composition described herein is present in a unit dose serving in a sachet in an amount of about 24 g.

In some embodiments, a dose of a composition of the disclosure can be expressed in terms of an amount of the drug divided by the mass of the subject, for example, grams of drug per kilograms of subject body mass. In some embodiments, a therapeutically-effective amount of a composition of the disclosure is from about 0.1 g/kg/day to about 0.2 g/kg/day, from about 0.2 g/kg/day to about 0.3 g/kg/day, from about 0.3 g/kg/day to about 0.4 g/kg/day, from about 0.4 g/kg/day to about 0.5 g/kg/day, from about 0.5 g/kg/day to about 0.6 g/kg/day, from about 0.6 g/kg/day to about 0.7 g/kg/day, from about 0.7 g/kg/day to about 0.8 g/kg/day, from about 0.8 g/kg/day to about 0.9 g/kg/day, from about 0.9 g/kg/day to about 1.0 g/kg/day, from about 1.0 g/kg/day to about 1.1 g/kg/day, from about 1.1 g/kg/day to about 1.2 g/kg/day, from about 1.2 g/kg/day to about 1.3 g/kg/day, from about 1.3 g/kg/day to about 1.4 g/kg/day, or from about 1.4 g/kg/day to about 1.5 g/kg/day. In some embodiments, a therapeutically-effective amount of a composition of the disclosure is from about 0.4 g/kg/day to about 0.5 g/kg/day. In some embodiments, a therapeutically-effective amount of a composition of the disclosure is from about 0.6 g/kg/day to about 0.7 g/kg/day. In some embodiments, a therapeutically-effective amount of a composition of the disclosure is from about 0.8 g/kg/day to about 0.9 g/kg/day.

In some embodiments, a dose of a composition of the disclosure can be expressed in terms of an amount of the drug divided by the mass of the subject, for example, milligrams of drug per kilograms of subject body mass. In some embodiments, a composition is provided in an amount ranging from about 100 mg/kg to about 150 mg/kg, about 150 mg/kg to about 200 mg/kg, about 200 mg/kg to about 250 mg/kg, about 250 mg/kg to about 300 mg/kg, or about 300 mg/kg to about 350 mg/kg. In some embodiments, a composition is provided in an amount of about 100 mg/kg, about 150 mg/kg, about 200 mg/kg, about 250 mg/kg, about 300 mg/kg, or about 350 mg/kg.

A composition described herein can be provided to a subject to achieve an amount of protein per body weight of the subject. In some embodiments, a composition described herein can be provided to a subject to achieve a range from about 0.2 g protein/kg to about 0.4 g protein/kg, about 0.4 g protein/kg to about 0.6 g protein/kg, about 0.6 g protein/kg to about 0.8 g protein/kg, or about 0.8 g protein/kg to about 1 g protein/kg of body weight of the subject. In some embodiments, a composition described herein can be provided to a subject to achieve a range from about 0.6 g protein/kg to about 0.8 g protein/kg of body weight of the subject.

A composition described herein can be provided to a subject in one or more servings per day. In some embodiments, 1 serving, 2 servings, 3 servings, 4 servings, 5 servings, 6 servings, 7 servings, 8 servings, 9 servings, 10 servings, 11 servings, or 12 servings of a composition described herein is provided to a subject in one day. In some embodiments, 3 servings of a composition described herein is provided to a subject in one day. In some embodiments, 6 servings of a composition described herein is provided to a subject in one day. In some embodiments, 9 servings of a composition described herein is provided to a subject in one day.

### Methods of Administration

A composition of the disclosure can be administered to a subject, and the administration can be accompanied by a food-based diet low in or substantially devoid of at least one amino acid. In some embodiments, administration of a composition of the disclosure is accompanied by a food-based diet low in or substantially devoid of one amino acid. In some embodiments, administration of a composition of the disclosure is accompanied by a food-based diet low in or substantially devoid of proline. In some embodiments, administration of a composition of the disclosure is accompanied by a food-based diet low in or substantially devoid of serine. In some embodiments, administration of a composition of the disclosure is accompanied by a food-based diet low in or substantially devoid of glycine. In some embodiments, administration of a composition of the disclosure is accompanied by a food-based diet low in or substantially devoid of two amino acids or salts thereof. In some embodiments, administration of a composition of the disclosure is accompanied by a food-based diet low in or substantially devoid of serine and glycine. In some embodiments, administration of a composition of the disclosure is accompanied by a food-based diet low in or substantially devoid of three amino acids or salts thereof. In some embodiments, administration of a composition of the disclosure is accompanied by a food-based diet low in or substantially devoid of serine, glycine, and proline. In some embodiments, administration of a composition of the disclosure is accompanied by a food-based diet low in or substantially devoid of serine, glycine, and cysteine. In some embodiments, administration of a composition of the disclosure is accompanied by a food-based diet low in or substantially devoid of four amino acids or salts thereof. A salt of an amino acid disclosed herein can be a pharmaceutically acceptable salt.

A composition of the disclosure can be administered to a subject that is on a diet. In some embodiments, a composition of the disclosure is administered to the subject, and the subject is on a diet that is low in protein. In some embodiments, a composition of the disclosure is administered to the subject, and the subject is on a low carbohydrate diet. In some embodiments, a composition of the disclosure is administered to the subject, and the subject is on a high-fat, and low-carbohydrate (e.g., ketogenic type diet). In some embodiments, a composition of the disclosure is administered to the subject, and the subject is on a vegetarian diet. In some embodiments, a composition of the disclosure is administered to the subject, and the subject is on a vegan diet.

In some embodiments, a composition of the disclosure is administered to a subject that is on a low protein diet designed to be low in at least one non-essential amino acid. In some embodiments, a composition of the disclosure is administered to a subject that is on a low protein diet designed to be low in serine and glycine. In some embodiments, a composition of the disclosure is administered to a subject that is on a low protein diet designed to be low in proline, serine, and glycine. In some embodiments, a composition of the disclosure is administered to a subject that is on a low protein diet with less than about 2 g/day, about 1.75 g/day, about 1.5 g/day, about 1.25 g/day, about 1 g/day, about 0.75 g/day, or about 0.5 g/day. In some embodiments, a composition of the disclosure is administered to a subject that is on a low protein diet with less than about 500 mg/day, about 450 mg/day, about 400 mg/day, about 350 mg/day, about 300 mg/day, about 250 mg/day, about 200 mg/day, about 150 mg/day, about 100 mg/day, or about 50 mg/day.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms.

A composition disclosed herein can be administered as soon as is practical after the onset of a disease or condition is detected or suspected, and for a length of time necessary for the treatment of the disease. In some embodiments, the length of time necessary for the treatment of disease is about 12 hours, about 24 hours, about 36 hours, or about 48 hours. In some embodiments, the length of time necessary for the treatment of disease is about 1 day, about 2 days, about 3 days, about 4 days, about 5 days, about 6 days, about 7 days, about 8 days, about 9 days, about 10 days, about 11 days, about 12 days, about 13 days, about 14 days, or about 15 days. In some embodiments, the length of time necessary for the treatment of disease is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks. In some embodiments, the length of time necessary for the treatment of disease is about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months.

In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

A composition of the disclosure can be administered alone continuously throughout a treatment period. In some embodiments, a composition of the disclosure can be administered simultaneously with administration of a therapeutic agent, for example, radiotherapy or chemotherapy. In some embodiments, a composition of the disclosure alone can be administered at least one time a day for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In some embodiments, a composition of the disclosure alone can be administered at least one time a day for about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, or about 1 year. In some embodiments, a composition of the disclosure can be administered at least one time a day for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days, and a therapeutic agent can be administered throughout the treatment period following an appropriate treatment regimen. In some embodiments, a composition of the disclosure can be administered at least one time a day for about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, or about 1 year, and a therapeutic agent can be administered throughout the treatment period following an appropriate treatment regimen.

A composition of the disclosure can be administered intermittently throughout a treatment period. In some embodiments, a subject can be treated with a composition of the disclosure during a treatment period, then go off treatment for an off-treatment period. In some embodiments, the length of the treatment period and off-treatment period are identical. In some embodiments, the length of the treatment period and off-treatment period are different. In some embodiments, the length of the treatment period is longer than the off-treatment period. In some embodiments, the length of the treatment period is shorter than the off-treatment period.

In some embodiments, the length of the treatment period is 2 days, 3 days, 4 days, 5 days, 6 days, 7 days, 8 days, 9 days, 10 days, 11 days, 12 days, 13 days, or 14 days. In some embodiments, the length of the treatment period is 5 days. In some embodiments, the length of the treatment period is 7 days. In some embodiments, the length of the treatment period is 10 days. In some embodiments, the length of the treatment period is 12 days. In some embodiments, the length of the treatment period is 14 days.

In some embodiments, the length of the off-treatment period is 2 days, 3 days, 4 days, 5 days, 6 days, 7 days, 8 days, 9 days, 10 days, 11 days, 12 days, 13 days, or 14 days. In some embodiments, the length of the off-treatment period is 2 days. In some embodiments, the length of the off-treatment period is 5 days.

In some embodiments, the length of the treatment period is 5 days, and the length of the off-treatment period is 2 days. In some embodiments, the length of the treatment period is 4 days, and the length of the off-treatment period is 3 days. In some embodiments, the length of the treatment period is 12 days, and the length of the off-treatment period is 2 days. In some embodiments, the length of the treatment period is 3 days, and the length of the off-treatment period is 4 days. In some embodiments, the length of the treatment period is 2 days, and the length of the off-treatment period is 5 days. In some embodiments, a cycle of a treatment period and an off-treatment period is repeated for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks.

In some embodiments, a subject can be treated with a composition of the disclosure and a therapeutic agent, then go off treatment before beginning a subsequent treatment cycle with the composition and the therapeutic agent. In some embodiments, the therapeutic agent is radiotherapy. In some embodiments, the therapeutic agent is radiotherapy. In some embodiments, the length of the treatment period and off-treatment period are identical. In some embodiments, the length of the treatment period and off-treatment period are different. In some embodiments, the length of the treatment period is longer than the off-treatment period. In some embodiments, the length of the treatment period is shorter than the off-treatment period.

In some embodiments, the length of the treatment period is 5 days, and the length of the off-treatment period is 2 days. In some embodiments, the length of the treatment period is 4 days, and the length of the off-treatment period is 3 days. In some embodiments, the length of the treatment period is 3 days, and the length of the off-treatment period is 4 days. In some embodiments, the length of the treatment period is 2 days, and the length of the off-treatment period is 5 days. In some embodiments, a cycle of a treatment period and an off-treatment period is repeated for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks.

A composition described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Aqueous suspension compositions can be packaged in single-dose non-reclosable containers. Multiple-dose reclosable containers can be used, for example, in combination with or without a preservative.

In some embodiments, a composition is administered to a subject throughout a day. In some embodiments, a composition is administered to a subject with a meal. In some embodiments, a composition is administered to a subject with a snack. In some embodiments, a composition is administered to a subject without a meal. In some embodiments, a composition is administered to a subject through the day in equal intervals. In some embodiments, a first serving is administered before breakfast, a second serving is administered with breakfast, a third serving is administered with lunch, a fourth and fifth serving is administered with dinner, and a sixth serving is administered before bed.

In some embodiments, the dietary product is administered 3 times a day. In some embodiments, the dietary product is administered 5 times a day.

A composition provided herein can be administered in conjunction with other therapies, for example, chemotherapy, radiation, surgery, anti-inflammatory agents, immunotherapy, biologicals, and selected vitamins. The other agents can be administered prior to, after, or concomitantly with the pharmaceutical compositions.

In some embodiments, a nutrient modulation therapy can be administered orally. For oral administration, a nutrient modulation therapy of the disclosure can be formulated by combining the active compounds (e.g., amino acids or salts thereof) with pharmaceutically-acceptable carriers or excipients. Such carriers can be used to formulate liquids, gels, syrups, elixirs, slurries, or suspensions, for oral ingestion by a subject. In some embodiments, a nutrient modulation therapy can be formulated as a powder. In some embodiments, a nutrient modulation therapy can be formulated as a beverage. In some embodiments, a nutrient modulation therapy can be formulated as a pill, a tablet, a suspension, a gel, a geltab, a semisolid, a tablet, a sachet, a lozenge, or a capsule or microcapsule. In other embodiments, a nutrient modulation therapy can be formulated as a liquid, a suspension, a gel, a geltab, a semisolid, a tablet, a sachet, a lozenge, or a capsule.

In some embodiments, a nutrient modulation therapy can be administered intravenously. The nutrient modulation therapy can be in a form suitable for parenteral injection as a sterile suspension, solution or emulsion in oily or aqueous vehicles, and can contain formulatory agents such as suspending, stabilizing and/or dispersing agents. Formulations for parenteral administration include aqueous solutions of the active compounds (e.g., amino acids or salts thereof) in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

### Methods of Use

Disclosed herein is a method comprising: a) isolating a biological sample from a subject, wherein the subject has a cancer; b) after the isolating, quantifying an amount of a biosynthetic metabolic enzyme; and c) after the quantifying, administering a nutrient modulation therapy to the subject.

In some embodiments, the method further comprises quantifying an amount of a second biosynthetic metabolic enzyme. In some embodiments, the method further comprises quantifying an amount of a second biosynthetic metabolic enzyme and an amount of a third biosynthetic metabolic enzyme. In some embodiments, the method further comprises, after the quantifying and before the administering, comparing the amount of the biosynthetic metabolic enzyme in the biological sample to a second amount of the biosynthetic metabolic enzyme in a reference biological sample, wherein the reference biological sample does not comprise a cancer cell. In some embodiments, the amount of the biosynthetic metabolic enzyme in the biological sample is lower than the second amount of the biosynthetic metabolic enzyme in the reference sample.

In some embodiments, the method further comprises, after the quantifying and before the administering, subjecting the biosynthetic metabolic enzyme to an empirical scoring system, wherein the empirical scoring system has a threshold value, wherein the biological sample having a sample value below the threshold value indicates a likelihood of the subject displaying a positive response to the nutrient modulation therapy. In some embodiments, the sample value is lower than the threshold value of the empirical scoring system.

Disclosed herein is a method comprising: a) isolating a biological sample from a subject, wherein the subject has a cancer; and b) after the isolating, quantifying an amount of a biosynthetic metabolic enzyme, wherein the amount of the biosynthetic metabolic enzyme predicts a likelihood of the subject displaying a positive response to a nutrient modulation therapy to treat the cancer.

In some embodiments, the nutrient modulation therapy comprises administering a therapeutically effective amount of a dietary product. In some embodiments, the dietary product is devoid of at least one amino acid. In some embodiments, the dietary product is devoid of serine. In some embodiments, the dietary product is devoid of glycine. In some embodiments, the dietary product is devoid of proline. In some embodiments, the dietary product is devoid of tyrosine. In some embodiments, the dietary product is devoid of at least two amino acids. In some embodiments, the dietary product is devoid of serine and glycine. In some embodiments, the dietary product is devoid of at least three amino acids. In some embodiments, the dietary product is devoid of serine, glycine, and proline. In some embodiments, the dietary product is devoid of serine, glycine, and cysteine.

In some embodiments, the method further comprises, after the quantifying, administering a cancer therapy to the subject. In some embodiments, the cancer therapy is radiotherapy. In some embodiments, the cancer therapy is chemotherapy.

In some embodiments, the method further comprises, after the quantifying, comparing the amount of the biosynthetic metabolic enzyme in the biological sample to a second amount of the biosynthetic metabolic enzyme in a reference biological sample, wherein the reference biological sample does not comprise a cancer cell. In some embodiments, the amount of the biosynthetic metabolic enzyme in the biological sample is lower than the second amount of the biosynthetic metabolic enzyme in the reference sample. In some embodiments, the method further comprises, after the quantifying, subjecting the amount of the first biosynthetic metabolic enzyme to an empirical scoring system, wherein the empirical scoring system has a threshold value, wherein the biological sample having a first sample value below the threshold value indicates a likelihood of the subject displaying a positive response to a nutrient modulation therapy. In some embodiments, the sample value is lower than the threshold value of the empirical scoring system. In some embodiments, the method further comprises, after the isolating, quantifying an amount of a second biosynthetic metabolic enzyme. In some embodiments, the method further comprises, after the isolating, quantifying an amount of a second biosynthetic metabolic enzyme and a third biosynthetic metabolic enzyme.

Disclosed herein is a method of treating a cancer in a subject in need thereof, the method comprising: a) administering a therapeutically effective amount of a dietary product, wherein the dietary product is devoid of at least serine and glycine; and b) administering a therapeutically effective amount of a therapeutic agent, wherein a cancer cell of the subject expresses low levels of at least one biosynthetic metabolic enzyme, wherein the at least one biosynthetic metabolic enzyme catalyzes the biosynthesis of serine or glycine, and wherein the administering the dietary product increases an efficacy of the therapeutic agent by at least about 20% as compared to an efficacy of the therapeutic agent in the absence of the dietary product. In some embodiments, the subject is human.

In some embodiments, the dietary product is further devoid of proline. In some embodiments, the dietary product is further devoid of tyrosine. In some embodiments, the dietary product is further devoid of cysteine. In some embodiments, the dietary product is further devoid of arginine. In some embodiments, the dietary product is further devoid of glutamine. In some embodiments, the dietary product is further devoid of glutamic acid or glutamate.

In some embodiments, the dose of the therapeutic agent in the presence of the dietary product is at least about 20% lower as compared to the dose of the therapeutic agent in the absence of the dietary product. In some embodiments, the dose of the therapeutic agent in the presence of the dietary product is at least about 40% lower as compared to the dose of the therapeutic agent in the absence of the dietary product. In some embodiments, the method decreases cancer cell proliferation in the subject by at least about 20% compared to a subject that is not administered the dietary product. In some embodiments, the method decreases cancer cell proliferation in the subject by at least about 40% compared to the subject that is not administered the dietary product. In some embodiments, the method decreases a tumor volume by at least about 20% compared to a subject that is not administered the dietary product. In some embodiments, the method decreases the tumor volume by at least about 40% compared to the subject that is not administered the dietary product. In some embodiments, the method decreases circulating levels of the at least two amino acids by at least about 20% compared to a subject that is not administered the dietary product. In some embodiments, the method decreases circulating levels of the at least two amino acids by at least about 40% compared to the subject that is not administered the dietary product. In some embodiments, the method decreases tissue levels of the at least two amino acids by at least about 20% compared to a subject that is not administered the dietary product. In some embodiments, the method decreases tissue levels of the at least two amino acids by at least about 40% compared to the subject that is not administered the dietary product.

In some embodiments, the administering the dietary product increases the efficacy of the therapeutic agent by at least about 40% as compared to the efficacy of the therapeutic agent in the absence of the dietary product.

In some embodiments, the method comprises administering the dietary product for a first period of time, and withholding the dietary product for a second period of time.

Disclosed herein is a method of treating a condition in a subject in need thereof, the method comprising administering a therapeutically effective amount of a dietary product, wherein the dietary product is devoid of at least serine, glycine, and proline.

In some embodiments, the dietary product comprises at least 12 amino acids. In some embodiments, the dietary product is further devoid of tyrosine. In some embodiments, the dietary product is further devoid of cysteine. In some embodiments, the dietary product is further devoid of arginine.

In some embodiments, the method further comprises administering a therapeutically effective amount of a therapeutic agent. In some embodiments, the administering the therapeutic agent is oral. In some embodiments, the administering the therapeutic agent is intravenous.

Disclosed herein is a pharmaceutical composition comprising in unit dosage form: a) a plurality of amino acids, wherein the composition is devoid of at least serine, glycine, and proline; and b) a pharmaceutically acceptable excipient. In some embodiments, the composition comprises at least 12 amino acids. In some embodiments, the composition is further devoid of cysteine. In some embodiments, the composition is further devoid of tyrosine. In some embodiments, the composition is further devoid of asparagine.

The present disclosure provides methods for treating a subject. A composition disclosed herein can be used in the treatment of any disease. In some embodiments, a composition disclosed herein is used to treat cancer in a subject in need thereof. Altering the diet and nutrient of a subject can have desired health benefits and can be efficacious in the treatment of disease.

Based on the particular disease and/or need of the patient, the present disclosure provides methods for generalized-treatment recommendation for a subject as well as methods for subject-specific treatment recommendation. Methods for treatments can comprise one of the following steps: determining a level of a nutrient in a subject; detecting a presence or absence of a disease in the subject based upon the determining, and recommending to the subject at least one generalized or subject-specific treatment to ameliorate disease symptoms.

In some embodiments, a composition disclosed herein can be used to manage a disease or condition by a dietary intervention. In some embodiments, a composition disclosed herein can be used as part of a treatment plan for a particular disease or condition.

In some embodiments, the subject has a cancer that expresses low levels of at least one enzyme. In some embodiments, the at least one enzyme catalyzes the biosynthesis of serine or glycine. In some embodiments, the at least one enzyme is phosphoglycerate dehydrogenase (PHGDH). In some embodiments, the at least one enzyme is phosphoserine aminotransferase 1 (PSAT1). In some embodiments, the enzyme is phosphoserine phosphatase (PSPH). In some embodiments, the at least one enzyme catalyzes the biosynthesis of glycine. In some embodiments, the enzyme is serine hydroxy methyltransferase 1 (SHMT1). In some embodiments, the enzyme is serine hydroxy methyltransferase 2 (SHMT2). In some embodiments, the enzyme is glycine synthase.

In some embodiments, the subject has cancer. Cancer is caused by uncontrollable growth of neoplastic cells, leading to invasion of adjacent and distant tissues resulting in death. Cancer cells often have underlying genetic or epigenetic abnormalities that affect both coding and regulatory regions of the genome. Genetic abnormalities in cancer cells can change protein structures, dynamic and expression levels, which in turn alter the cellular metabolism of the cancer cells. Changes in cell cycles can make cancer cells proliferate at a much higher speed than normal cells. With the increased metabolic rate and proliferation, cancer tissues have much higher nutrient demands compared to normal tissues.

Cancer cells have nutrient auxotrophy and have a much higher nutrient demand compared to normal cells. As an adaptation to fulfill the increased nutritional demand, cancer cells can up-regulate the glucose and amino acid transporters on the cell membrane to obtain more nutrients from circulation. Cancer cells can also rewire metabolic pathways by enhancing glycolysis and glutaminolysis to sustain a higher rate of ATP production or energy supply. Glucose and amino acids are highly demanded nutrients in cancer cells. Some cancer cell types and tumor tissues are known to be auxotrophic to specific amino acids. Cancers' auxotrophy to different amino acids can render the cancer types vulnerable to amino acid starvation treatments.

When mammalian cells experience amino acid starvation, the cells undergo a homeostatic response to amino acid shortage. Amino acid deficiency can trigger a general amino acid control pathway that involves shifting resources and energy of cells to expression of membrane trans-porters, growth hormones, and metabolic enzymes for amino acid homeostasis. Up-regulation of membrane transporters can enhance amino acid uptake, and up-regulation of metabolic enzymes can enhance amino acid synthesis. The cells can also recycle proteins and organelles to regenerate non-essential amino acids by autophagy. By general amino acid control pathway and autophagy, cells attempt to maintain amino acid homeostasis. Tumor tissues can also overcome amino acid starvation by enhancing angiogenesis to obtain more nutrient supply.

When homeostasis cannot be achieved upon severe amino acid starvation, cancer cells can inhibit protein synthesis, suppress growth, or undergo programmed cell death. The cell death mechanisms of amino acid starvation can be caspase-dependent apoptosis, autophagic cell death, or ferroptotic cell death. Amino acid transporters, metabolic enzymes, autophagy-associated proteins, and amino acid starvation can be used to control cancer growth.

A method disclosed herein can monitor nutrient consumption by a subject. The nutrient consumption can be measured by taking a biological sample from a subject. The biological sample can be for example, whole blood, serum, plasma, mucosa, saliva, cheek swab, urine, stool, cells, tissue, bodily fluid, sweat, breath, lymph fluid, CNS fluid, and lesion exudates. A combination of biological samples can be used with the methods of the disclosure.

A method of composition of the disclosure can slow the proliferation of cancer cell lines, or kill cancer cells. Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative dis-orders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

In some embodiments, the cancer is pancreatic cancer. In some embodiments, the cancer is breast cancer. In some embodiments, the cancer is ovarian cancer. In some embodiments, the cancer is colon cancer. In some embodiments, the cancer is rectal cancer. In some embodiments, the cancer is colorectal cancer. In some embodiments, the cancer is metastatic colorectal cancer. In some embodiments, the cancer is pancreatic cancer. In some embodiments, the cancer is metastatic pancreatic cancer.

In some embodiments, a composition of the disclosure can be used to treat a disease of tissue overgrowth or tissue over proliferation. In some embodiments, a composition of the disclosure can be used to treat excessive collagen synthesis. In some embodiments, a composition of the disclosure can be used to treat a skin condition, for example, a skin condition with an overgrowth of cells.

In some embodiments, a composition of the disclosure can be administered with a high fat diet. In some embodiments, the high fat diet is a diet that has greater than about 50%, about 60%, about 70%, about 80%, or about 90% daily calories from fat. In some embodiments, a composition of the disclosure is administered with a low carbohydrate diet. In some embodiments, the low carbohydrate diet is a diet with less than about 50%, about 40%, about 30%, about 20%, about 10%, or about 5% daily calories from carbohydrates. In some embodiments, a composition of the disclosure is administered with a low protein diet. In some embodiments, the low protein diet is a diet with less than about 15%, about 14%, about 13%, about 12%, about 11%, about 10%, about 9%, about 8%, about 7%, about 6%, about 5%, about 4%, about 3%, about 2%, or about 1% of daily calories from whole protein. In some embodiments, the low protein diet has a whole protein amount of less than about 50 g/day, about 40 g/day, about 30 g/day, about 20 g/day, or about 10 g/day. In some embodiments, a composition of the disclosure is administered with a high fat, low carbohydrate, and low protein diet. In some embodiments, a composition of the disclosure is administered with a normal diet.

### Combination Therapy

In some embodiments, a method of the disclosure comprises administering a therapeutic agent. In some embodiments, the therapeutic agent is an inhibitor of an enzyme involved in the synthesis and processing of a nutrient. In some embodiments, the therapeutic agent is a small molecule inhibitor. In some embodiments, the therapeutic agent is a therapeutic antibody, for example, an antibody that promotes degradation of a target enzyme.

Multiple therapeutic agents can be administered in any order or simultaneously. In some embodiments, a dietary product of the disclosure is administered in combination with, before, or after treatment with another therapeutic agent. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 h of the onset of the symptoms, within the first 24 h of the onset of the symptoms, within the first 6 h of the onset of the symptoms, or within 3 h of the onset of the symptoms. The initial administration can be via any route practical, such as by any route described herein using any formulation described herein.

In some embodiments, the method comprises administering two therapeutic agents. In some embodiments, the two therapeutic agents are each independently chemotherapeutic agents. In some embodiments, the two therapeutic agents are gemcitabine and paclitaxel. In some embodiments, the method comprises administering at least three therapeutic agents. In some embodiments, the three therapeutic agents are folinic acid or a pharmaceutically acceptable salt thereof, 5-fluorouracil or a pharmaceutically acceptable salt thereof, and irinotecan or a pharmaceutically acceptable salt thereof. In some embodiments, the method comprises administering four therapeutic agents. In some embodiments, the four therapeutic agents are folinic acid or a pharmaceutically acceptable salt thereof, 5-fluorouracil or a pharmaceutically acceptable salt thereof, oxaliplatin, and irinotecan or a pharmaceutically acceptable salt thereof. In some embodiments, the four therapeutic agents are folinic acid or a pharmaceutically acceptable salt thereof, 5-fluorouracil or a pharmaceutically acceptable salt thereof, irinotecan or a pharmaceutically acceptable salt thereof, and bevacizumab.

In some embodiments, the method further comprises administering a ketogenic diet.

### A) Radiation Therapy

Radiation therapy, or radiotherapy, is a therapy using ionizing radiation as a part of cancer treatment to control or kill malignant cells and is normally delivered by a linear accelerator. Ionizing radiation damages the DNA of cancerous tissue, resulting in cellular death. Radiation therapy can be curative in a number of types of cancer if localized to one area of the body. In some embodiments, the methods and compositions of the disclosure can be administered in combination with a second therapy, for example, radiotherapy. In some embodiments, radiotherapy can be used with a method or composition of the disclosure because radiotherapy can control cell growth.

In some embodiments, radiotherapy can be used in combination with a method or composition of the disclosure to prevent or reduce the likelihood of tumor recurrence after surgery to remove a primary malignant tumor. In some embodiments, radiotherapy and chemotherapy can be used in combination with a method or composition of the disclosure. In some embodiments, the methods and compositions of the disclosure can be administered in combination with radiotherapy to treat a cancer. In some embodiments, the methods and compositions of the disclosure can be administered in combination with radiotherapy to reduce symptoms of a cancer. In some embodiments, the methods and compositions of the disclosure can be administered in combination with radiotherapy to slow the growth of a cancer.

In some embodiments, the radiotherapy is external beam radiation therapy. External beam radiation therapy uses a machine that locally aims radiation at a cancer. In some embodiments, the radiotherapy is internal beam radiation therapy. In some embodiments, external beam radiation can be used to shrink tumors to treat pain, trouble breathing, or loss of bowel or bladder control. In some embodiments, the external-beam radiation therapy is three-dimensional conformal radiation therapy (3D-CRT). In some embodiments, the external-beam radiation therapy is intensity modulated radiation therapy (IMRT). In some embodiments, the external-beam radiation therapy is proton beam therapy. In some embodiments, the external-beam radiation therapy is image-guided radiation therapy (IGRT). In some embodiments, the external-beam radiation therapy is stereotactic radiation therapy (SRT).

Internal radiation therapy is a treatment that places a source of radiation in the subject's body. In some embodiments, the source of radiation is a liquid. In some embodiments, the source of radiation is a solid. In some embodiments, the internal radiotherapy uses a permanent implant. In some embodiments, the internal radiotherapy is a temporary internal radiotherapy, for example, a needle, tube, or applicator. In some embodiments, the solid source of radiation is used in brachytherapy. In some embodiments, seeds, ribbons, or capsules containing a radiation source are placed in a subject's body. In some embodiments, the radiotherapy is brachytherapy, where a radioactive source is placed inside or next to an area requiring treatment. In some embodiments, the radiotherapy is total body irradiation (TBI) in preparation for a bone marrow transplant.

In some embodiments, the radiotherapy is intraoperative radiation therapy (IORT). In some embodiments, the radiotherapy is systemic radiation therapy. In some embodiments, the radiotherapy is radioimmunotherapy. In some embodiments, the radiotherapy uses a radiosensitizer or a radioprotector.

In some embodiments, brachytherapy is used to treat a cancer of the head, neck, breast, cervix, prostate, or eye. In some embodiments, a systemic radiation therapy such as radioactive iodine, or I-131, can be used to treat thyroid cancer. In some embodiments, targeted radionuclide therapy can be used to treat advanced prostate cancer or a gastroenteropancreatic neuroendocrine tumor (GEP-NET).

In some embodiments, shaped radiation beams can be aimed from several angles of exposure to intersect at the tumor while sparing normal tissue. In some embodiments, a tumor absorbs a much larger dose of radiation than does a surrounding healthy tissue.

In some embodiments, a subject or tumor can be treated with about 0.5 Gray (Gy), about 1 Gy, about 1.5 Gy, about 2 Gy, about 2.5 Gy, about 3 Gy, about 3.5 Gy, about 4 Gy, about 4.5 Gy, about 5 Gy, about 5.5 Gy, about 6 Gy, about 6.5 Gy, about 7 Gy, about 7.5 Gy, about 8 Gy, about 8.5 Gy, about 9 Gy, about 9.5 Gy, or about 10 Gy. In some embodiments, a subject or tumor can be treated with about 5 Gy, about 10 Gy, about 15 Gy, about 20 Gy, about 25 Gy, about 30 Gy, about 35 Gy, about 40 Gy, about 45 Gy, about 50 Gy, about 55 Gy, about 60 Gy, about 65 Gy, about 70 Gy, about 75 Gy, about 80 Gy, about 85 Gy, about 90 Gy, about 95 Gy, or about 100 Gy of radiation therapy. In some embodiments, a subject or tumor can be treated with about 5 Gy of radiation therapy. In some embodiments, a subject or tumor can be treated with about 10 Gy of radiation therapy. In some embodiments, a subject or tumor can be treated with about 20 Gy of radiation therapy.

In some embodiments a subject or tumor can be treated with from about 5 Gy to about 10 Gy; about 10 Gy to about 15 Gy; about 15 Gy to about 20 Gy; about 20 Gy to about 25 Gy; about 25 Gy to about 30 Gy; about 30 Gy to about 35 Gy; about 35 Gy to about 40 Gy; about 40 Gy to about 45 Gy; about 45 Gy to about 50 Gy; about 50 Gy to about 55 Gy; about 55 Gy to about 60 Gy; about 60 Gy to about 65 Gy; about 65 Gy to about 70 Gy; about 70 Gy to about 75 Gy; or about 75 Gy to about 80 Gy. In some embodiments a subject or tumor can be treated with from about 5 Gy to about 10 Gy. In some embodiments a subject or tumor can be treated with from about 20 Gy to about 40 Gy. In some embodiments a subject or tumor can be treated with from about 40 Gy to about 60 Gy.

In some embodiments, one cycle of radiation therapy can comprise the subject or tumor being treated with radiation over a number of days. In some embodiments, the radiation can occur over 1 day, 2 days, 3 days, 4 days, 5 days, 6 days, 7 days, 8 days, 9 days, 10 days, 11 days, 12 days, 13 days, or 14 days. In some embodiments, one cycle of radiation therapy can comprise the subject or tumor being treated with radiation over 4 days. In some embodiments, one cycle of radiation therapy can comprise the subject or tumor being treated with radiation over 5 days.

In some embodiments, one cycle of radiation can comprise administering 10 Gy over 5 days, for example, 2 Gy a day for 5 days. In some embodiments, one cycle of radiation can comprise administering 15 Gy over 5 days, for example, 3 Gy a day for 5 days. In some embodiments, one cycle of radiation can comprise administering 20 Gy over 5 days, for example, 4 Gy a day for 5 days. In some embodiments, one cycle of radiation can comprise administering 25 Gy over 5 days, for example, 5 Gy a day for 5 days.

In some embodiments, one cycle of radiation therapy can be repeated over a period of time. In some embodiments, a cycle of radiation therapy can be repeated for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks.

In some embodiments, a composition of the disclosure can be administered simultaneously with administration of a radiotherapy. In some embodiments, a composition of the disclosure can be administered simultaneously with a radiotherapy for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In some embodiments, a composition of the disclosure can be administered simultaneously with administration of a radiotherapy for 5 days. In some embodiments, a composition of the disclosure can be administered simultaneously with administration of a radiotherapy for 7 days.

In some embodiments, the composition of the disclosure is administered 1 day, 2 days, 3 days, 4 days, 5 days, 6 days, 7 days, 8 days, 9 days, 10 days, 11 days, 12 days, 13 days, or 14 days before a subject is treated with radiotherapy. In some embodiments, the composition of the disclosure is administered 1 day before a subject is treated with radiotherapy. In some embodiments, the composition of the disclosure is administered 2 days before a subject is treated with radiotherapy. In some embodiments, the composition of the disclosure is administered 3 days before a subject is treated with radiotherapy. In some embodiments, the composition of the disclosure is administered 4 days before a subject is treated with radiotherapy.

In some embodiments, a subject can be treated with a composition of the disclosure and radiotherapy, then go off treatment before beginning a subsequent treatment cycle with the composition and radiotherapy. In some embodiments, the length of the treatment period and off-treatment period are identical. In some embodiments, the length of the treatment period and off-treatment period are different. In some embodiments, the length of the treatment period is longer than the off-treatment period. In some embodiments, the length of the treatment period is shorter than the off-treatment period.

In some embodiments, the length of a treatment period with a composition and radio therapy is 1 day, 2 days, 3 days, 4 days, 5 days, 6 days, 7 days, 8 days, 9 days, 10 days, 11 days, 12 days, 13 days, or 14 days, and the length of off-treatment period is 1 day, 2 days, 3 days, 4 days, 5 days, 6 days, 7 days, 8 days, 9 days, 10 days, 11 days, 12 days, 13 days, or 14 days. In some embodiments, the length of the treatment period is 5 days, and the length of the off-treatment period is 2 days. In some embodiments, the length of the treatment period is 4 days, and the length of the off-treatment period is 3 days. In some embodiments, the length of the treatment period is 3 days, and the length of the off-treatment period is 4 days. In some embodiments, the length of the treatment period is 2 days, and the length of the off-treatment period is 5 days.

In some embodiments, a cycle of a treatment period and an off-treatment period is repeated for 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks.

In some embodiments, a composition of the disclosure and radiotherapy are administered with a high fat diet. In some embodiments, the high fat diet is a diet that has greater than about 50%, about 60%, about 70%, about 80%, or about 90% daily calories from fat. In some embodiments, a composition of the disclosure and radiotherapy are administered with a low carbohydrate diet. In some embodiments, the low carbohydrate diet is a diet with less than about 50%, about 40%, about 30%, about 20%, about 10%, or about 5% daily calories from carbohydrates. In some embodiments, a composition of the disclosure and radiotherapy are administered with a low protein diet. In some embodiments, the low protein diet is a diet with less than about 15%, about 14%, about 13%, about 12%, about 11%, about 10%, about 9%, about 8%, about 7%, about 6%, about 5%, about 4%, about 3%, about 2%, or about 1% of daily calories from whole protein. In some embodiments, the low protein diet has a whole protein amount of less than about 50 g/day, about 40 g/day, about 30 g/day, about 20 g/day, or about 10 g/day. In some embodiments, a composition of the disclosure and radiotherapy are administered with a high fat, low carbohydrate, and low protein diet.

### B) Chemotherapy

In some embodiments, an amino acid starvation therapy of the disclosure can be used in combination with a chemotherapeutic regimen. In some embodiments, the chemotherapeutic regimen is treatment with a chemotherapy agent. In some embodiments, the chemotherapy agent is Gemcitabine, Paclitaxel, fluorouracil (5FU) or a pharmaceutically acceptable salt thereof, Oxaliplatin, Folinic acid or a pharmaceutically acceptable salt thereof, Irinotecan or a pharmaceutically acceptable salt thereof, or combinations of two or more thereof. In some embodiments, the chemotherapy agent is an alkylating agent. In some embodiments, the alkylating agent is altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, or trabectedin. In some embodiments, the alkylating agent is a nitrosoureas, for example, carmustine, lomustine, or streptozocin.

In some embodiments, the chemotherapy agent is an antimetabolite. In some embodiments, the antimetabolite is azacytidine, 5FU, 6-mercaptopurine (6-MP), capecitabine (Xeloda), cladribine, clofarabine, cytarabine (Ara-C), decitabine, floxuridine, fludarabine, gemcitabine (Gemzar), hydroxyurea, methotrexate, nelarabine, pemetrexed (Alimta), pentostatin, pralatrexate, thioguanine, or a combination of trifluridine and tipiracil.

In some embodiments, the chemotherapy agent is an anti-tumor antibiotic. In some embodiments, the anti-tumor antibiotic is an anthracycline. In some embodiments, the anthracycline is daunorubicin, doxorubicin (Adriamycin), epirubicin, idarubicin, or valrubicin. In some embodiments, the anti-tumor antibiotic is not an anthracycline. In some embodiments, the anti-tumor antibiotic is bleomycin, dactinomycin, mitomycin-C, or mitoxantrone.

In some embodiments, the chemotherapy agent is a topoisomerase inhibitor. In some embodiments, the chemotherapy agent is a topoisomerase I inhibitor, for example, irinotecan or a pharmaceutically acceptable salt thereof, irinotecan liposomal, or topotecan. In some embodiments, the chemotherapy agent is a topoisomerase II inhibitor, for example, etoposide, mitoxantrone, or teniposide. In some embodiments, the chemotherapy agent is a mitotic inhibitor. In some embodiments, the mitotic inhibitor is a taxane, for example, vinblastine, vincristine, vincristine liposomal, or vinorelbine. In some embodiments, the chemotherapy agent is a corticosteroid, for example, prednisone, methylprednisolone, or dexamethasone.

In some embodiments, the chemotherapy agent is all-trans-retinoic acid, arsenic trioxide, asparaginase, eribulin, hydroxyurea, ixabepilone, mitotane, omacetaxine, pegaspargase, procarbazine, romidepsin, or vorinostat.

In some embodiments, the dose of the chemotherapy agent is about 10 mg/m² to about 1,000 mg/m². In some embodiments, the dose of the chemotherapy agent is about 10 mg/m² to about 50 mg/m², about 10 mg/m² to about 100 mg/m², about 10 mg/m² to about 200 mg/m², about 10 mg/m² to about 300 mg/m², about 10 mg/m² to about 400 mg/m², about 10 mg/m² to about 500 mg/m², about 10 mg/m² to about 600 mg/m², about 10 mg/m² to about 700 mg/m², about 10 mg/m² to about 800 mg/m², about 10 mg/m² to about 900 mg/m², about 10 mg/m² to about 1,000 mg/m², about 50 mg/m² to about 100 mg/m², about 50 mg/m² to about 200 mg/m², about 50 mg/m² to about 300 mg/m², about 50 mg/m² to about 400 mg/m², about 50 mg/m² to about 500 mg/m², about 50 mg/m² to about 600 mg/m², about 50 mg/m² to about 700 mg/m², about 50 mg/m² to about 800 mg/m², about 50 mg/m² to about 900 mg/m², about 50 mg/m² to about 1,000 mg/m², about 100 mg/m² to about 200 mg/m², about 100 mg/m² to about 300 mg/m², about 100 mg/m² to about 400 mg/m², about 100 mg/m² to about 500 mg/m², about 100 mg/m² to about 600 mg/m², about 100 mg/m² to about 700 mg/m², about 100 mg/m² to about 800 mg/m², about 100 mg/m² to about 900 mg/m², about 100 mg/m² to about 1,000 mg/m², about 200 mg/m² to about 300 mg/m², about 200 mg/m² to about 400 mg/m², about 200 mg/m² to about 500 mg/m², about 200 mg/m² to about 600 mg/m², about 200 mg/m² to about 700 mg/m², about 200 mg/m² to about 800 mg/m², about 200 mg/m² to about 900 mg/m², about 200 mg/m² to about 1,000 mg/m², about 300 mg/m² to about 400 mg/m², about 300 mg/m² to about 500 mg/m², about 300 mg/m² to about 600 mg/m², about 300 mg/m² to about 700 mg/m², about 300 mg/m² to about 800 mg/m², about 300 mg/m² to about 900 mg/m², about 300 mg/m² to about 1,000 mg/m², about 400 mg/m² to about 500 mg/m², about 400 mg/m² to about 600 mg/m², about 400 mg/m² to about 700 mg/m², about 400 mg/m² to about 800 mg/m², about 400 mg/m² to about 900 mg/m², about 400 mg/m² to about 1,000 mg/m², about 500 mg/m² to about 600 mg/m², about 500 mg/m² to about 700 mg/m², about 500 mg/m² to about 800 mg/m², about 500 mg/m² to about 900 mg/m², about 500 mg/m² to about 1,000 mg/m², about 600 mg/m² to about 700 mg/m², about 600 mg/m² to about 800 mg/m², about 600 mg/m² to about 900 mg/m², about 600 mg/m² to about 1,000 mg/m², about 700 mg/m² to about 800 mg/m², about 700 mg/m² to about 900 mg/m², about 700 mg/m² to about 1,000 mg/m², about 800 mg/m² to about 900 mg/m², about 800 mg/m² to about 1,000 mg/m², or about 900 mg/m² to about 1,000 mg/m². In some embodiments, the dose of the chemotherapy agent is about 10 mg/m², about 50 mg/m², about 100 mg/m², about 200 mg/m², about 300 mg/m², about 400 mg/m², about 500 mg/m², about 600 mg/m², about 700 mg/m², about 800 mg/m², about 900 mg/m², or about 1,000 mg/m². In some embodiments, the dose of the chemotherapy agent is at least about 10 mg/m², about 50 mg/m², about 100 mg/m², about 200 mg/m², about 300 mg/m², about 400 mg/m², about 500 mg/m², about 600 mg/m², about 700 mg/m², about 800 mg/m², or about 900 mg/m². In some embodiments, the dose of the chemotherapy agent is at most about 50 mg/m², about 100 mg/m², about 200 mg/m², about 300 mg/m², about 400 mg/m², about 500 mg/m², about 600 mg/m², about 700 mg/m², about 800 mg/m², about 900 mg/m², or about 1,000 mg/m².

In some embodiments, the dose of the chemotherapy agent is about 0.1 mg/kg to about 15 mg/kg. In some embodiments, the dose of the chemotherapy agent is about 0.1 mg/kg to about 0.25 mg/kg, about 0.1 mg/kg to about 0.5 mg/kg, about 0.1 mg/kg to about 0.75 mg/kg, about 0.1 mg/kg to about 1 mg/kg, about 0.1 mg/kg to about 3 mg/kg, about 0.1 mg/kg to about 5 mg/kg, about 0.1 mg/kg to about 7 mg/kg, about 0.1 mg/kg to about 9 mg/kg, about 0.1 mg/kg to about 11 mg/kg, about 0.1 mg/kg to about 13 mg/kg, about 0.1 mg/kg to about 15 mg/kg, about 0.25 mg/kg to about 0.5 mg/kg, about 0.25 mg/kg to about 0.75 mg/kg, about 0.25 mg/kg to about 1 mg/kg, about 0.25 mg/kg to about 3 mg/kg, about 0.25 mg/kg to about 5 mg/kg, about 0.25 mg/kg to about 7 mg/kg, about 0.25 mg/kg to about 9 mg/kg, about 0.25 mg/kg to about 11 mg/kg, about 0.25 mg/kg to about 13 mg/kg, about 0.25 mg/kg to about 15 mg/kg, about 0.5 mg/kg to about 0.75 mg/kg, about 0.5 mg/kg to about 1 mg/kg, about 0.5 mg/kg to about 3 mg/kg, about 0.5 mg/kg to about 5 mg/kg, about 0.5 mg/kg to about 7 mg/kg, about 0.5 mg/kg to about 9 mg/kg, about 0.5 mg/kg to about 11 mg/kg, about 0.5 mg/kg to about 13 mg/kg, about 0.5 mg/kg to about 15 mg/kg, about 0.75 mg/kg to about 1 mg/kg, about 0.75 mg/kg to about 3 mg/kg, about 0.75 mg/kg to about 5 mg/kg, about 0.75 mg/kg to about 7 mg/kg, about 0.75 mg/kg to about 9 mg/kg, about 0.75 mg/kg to about 11 mg/kg, about 0.75 mg/kg to about 13 mg/kg, about 0.75 mg/kg to about 15 mg/kg, about 1 mg/kg to about 3 mg/kg, about 1 mg/kg to about 5 mg/kg, about 1 mg/kg to about 7 mg/kg, about 1 mg/kg to about 9 mg/kg, about 1 mg/kg to about 11 mg/kg, about 1 mg/kg to about 13 mg/kg, about 1 mg/kg to about 15 mg/kg, about 3 mg/kg to about 5 mg/kg, about 3 mg/kg to about 7 mg/kg, about 3 mg/kg to about 9 mg/kg, about 3 mg/kg to about 11 mg/kg, about 3 mg/kg to about 13 mg/kg, about 3 mg/kg to about 15 mg/kg, about 5 mg/kg to about 7 mg/kg, about 5 mg/kg to about 9 mg/kg, about 5 mg/kg to about 11 mg/kg, about 5 mg/kg to about 13 mg/kg, about 5 mg/kg to about 15 mg/kg, about 7 mg/kg to about 9 mg/kg, about 7 mg/kg to about 11 mg/kg, about 7 mg/kg to about 13 mg/kg, about 7 mg/kg to about 15 mg/kg, about 9 mg/kg to about 11 mg/kg, about 9 mg/kg to about 13 mg/kg, about 9 mg/kg to about 15 mg/kg, about 11 mg/kg to about 13 mg/kg, about 11 mg/kg to about 15 mg/kg, or about 13 mg/kg to about 15 mg/kg. In some embodiments, the dose of the chemotherapy agent is about 0.1 mg/kg, about 0.25 mg/kg, about 0.5 mg/kg, about 0.75 mg/kg, about 1 mg/kg, about 3 mg/kg, about 5 mg/kg, about 7 mg/kg, about 9 mg/kg, about 11 mg/kg, about 13 mg/kg, or about 15 mg/kg. In some embodiments, the dose of the chemotherapy agent is at least about 0.1 mg/kg, about 0.25 mg/kg, about 0.5 mg/kg, about 0.75 mg/kg, about 1 mg/kg, about 3 mg/kg, about 5 mg/kg, about 7 mg/kg, about 9 mg/kg, about 11 mg/kg, or about 13 mg/kg. In some embodiments, the dose of the chemotherapy agent is at most about 0.25 mg/kg, about 0.5 mg/kg, about 0.75 mg/kg, about 1 mg/kg, about 3 mg/kg, about 5 mg/kg, about 7 mg/kg, about 9 mg/kg, about 11 mg/kg, about 13 mg/kg, or about 15 mg/kg.

In some embodiments, a composition of the disclosure can be administered with gemcitabine. In some embodiments, a composition of the disclosure can be administered with from about 500 mg/m² to about 2000 mg/m² of gemcitabine. In some embodiments, a composition of the disclosure can be administered with from about 500 mg/m² to about 750 mg/m², from about 750 mg/m² to about 1000 mg/m², from about 1000 mg/m² to about 1250 mg/m², from about 1250 mg/m² to about 1500 mg/m², from about 1500 mg/m² to about 1750 mg/m², or from about 1750 mg/m² to about 2000 mg/m² of gemcitabine. In some embodiments, a composition of the disclosure can be administered with less than about 500 mg/m², about 750 mg/m², about 1000 mg/m², about 1250 mg/m², about 1500 mg/m², about 1750 mg/m², or about 2000 mg/m² of gemcitabine. In some embodiments, a composition of the disclosure can be administered with less than about 750 mg/m² of gemcitabine. In some embodiments, a composition of the disclosure can be administered with less than about 1000 mg/m² of gemcitabine. In some embodiments, a composition of the disclosure can be administered with less than about 1250 mg/m² of gemcitabine.

In some embodiments, a composition of the disclosure can be administered with paclitaxel. In some embodiments, a composition of the disclosure can be administered with from about 50 mg/m² to about 500 mg/mg² of paclitaxel. In some embodiments, a composition of the disclosure can be administered with from about 50 mg/m² to about 75 mg/mg², from about 75 mg/m² to about 100 mg/mg², from about 100 mg/m² to about 125 mg/mg², from about 125 mg/m² to about 150 mg/mg², from about 150 mg/m² to about 175 mg/mg², from about 175 mg/m² to about 200 mg/mg², from about 200 mg/m² to about 225 mg/mg², from about 225 mg/m² to about 250 mg/mg², from about 250 mg/m² to about 300 mg/mg², from about 300 mg/m² to about 325 mg/mg², from about 325 mg/m² to about 350 mg/mg², from about 350 mg/m² to about 375 mg/mg², from about 375 mg/m² to about 400 mg/mg², from about 400 mg/m² to about 425 mg/mg², from about 425 mg/m² to about 450 mg/mg², from about 450 mg/m² to about 475 mg/mg², or from about 475 mg/m² to about 500 mg/mg² of paclitaxel. In some embodiments, a composition of the disclosure can be administered with less than about 50 mg/m², about 100 mg/m², about 150 mg/m², about 200 mg/m², about 250 mg/m², about 300 mg/m², about 350 mg/m², about 400 mg/m², about 450 mg/m², or about 500 mg/m² of paclitaxel. In some embodiments, a composition of the disclosure can be administered with less than about 125 mg/m² of paclitaxel. In some embodiments, a composition of the disclosure can be administered with less than about 150 mg/m² of paclitaxel. In some embodiments, a composition of the disclosure can be administered with less than about 175 mg/m² of paclitaxel. In some embodiments, a composition of the disclosure can be administered with less than about 200 mg/m² of paclitaxel.

In some embodiments, a composition of the disclosure can be administered with gemcitabine and paclitaxel. In some embodiments, a composition of the disclosure can be administered with from about 500 mg/m² to about 2000 mg/m² of gemcitabine and from about 50 mg/m² to about 500 mg/mg² of paclitaxel. In some embodiments, a composition of the disclosure can be administered with from about 500 mg/m² to about 750 mg/m², from about 750 mg/m² to about 1000 mg/m², from about 1000 mg/m² to about 1500 mg/m², or from about 1500 mg/m² to about 2000 mg/m² of gemcitabine and from about 50 mg/m² to about 100 mg/mg², from about 100 mg/m² to about 150 mg/mg², from about 150 mg/m² to about 200 mg/mg², from about 200 mg/m² to about 250 mg/mg², from about 250 mg/m² to about 300 mg/mg², from about 300 mg/m² to about 350 mg/mg², from about 350 mg/m² to about 400 mg/mg², from about 400 mg/m² to about 450 mg/mg², or from about 450 mg/m² to about 500 mg/mg² of paclitaxel. In some embodiments, a composition of the disclosure can be administered with less than about 750 mg/m² of gemcitabine and less than about 150 mg/mg² of paclitaxel. In some embodiments, a composition of the disclosure can be administered with less than about 1000 mg/m² of gemcitabine and less than about 175 mg/mg² of paclitaxel. In some embodiments, a composition of the disclosure can be administered with less than about 1250 mg/m² of gemcitabine and less than about 200 mg/mg² of paclitaxel.

In some embodiments, the therapeutic agent is gemcitabine. In some embodiments, the therapeutically effective amount of gemcitabine is about 1000 mg/m². In some embodiments, the therapeutically effective amount of gemcitabine is less than about 1000 mg/m². In some embodiments, the therapeutic agent is paclitaxel. In some embodiments, the therapeutically effective amount of paclitaxel is about 175 mg/m². In some embodiments, the therapeutically effective amount of paclitaxel is less than about 175 mg/m².

In some embodiments, a composition of the disclosure can be administered with FOLFIRINOX, which comprises folinic acid or a pharmaceutically acceptable salt thereof, 5-fluorouracil or a pharmaceutically acceptable salt thereof, oxaliplatin, and irinotecan or a pharmaceutically acceptable salt thereof. In some embodiments, a composition of the disclosure can be administered with FOLFIRINOX comprising: 85 mg/m² oxaliplatin, 180 mg/m² irinotecan, 400 mg/m² folinic acid or a pharmaceutically acceptable salt thereof (also called leucovorin or calcium folinate), and 400 mg/m² fluorouracil. In some embodiments, a composition of the disclosure can be administered with FOLFIRINOX comprising: 85 mg/m² oxaliplatin, 180 mg/m² irinotecan or a pharmaceutically acceptable salt thereof, 400 mg/m² leucovorin or a pharmaceutically acceptable salt thereof, and 400 mg/m² fluorouracil or a pharmaceutically acceptable salt thereof as a bolus, followed by 2400 mg/m² given as a 46 hour continuous infusion every 2 weeks. In some embodiments, a composition of the disclosure can be administered with FOLFIRINOX comprising dosages of oxaliplatin, irinotecan or a pharmaceutically acceptable salt thereof, leucovorin or a pharmaceutically acceptable salt thereof, and fluorouracil or a pharmaceutically acceptable salt thereof typically used in treating a specific cancer type.

In some embodiments, a composition of the disclosure can be administered with a PI3K inhibitor, for example, serabelisib. In some embodiments, a composition of the disclosure can be administered with from about 30 mg/kg to about 150 mg/kg of serabelisib. In some embodiments, a composition of the disclosure can be administered with from about 30 mg/kg to about 50 mg/kg, from about 50 mg/kg to about 75 mg/kg, from about 75 mg/kg to about 100 mg/kg, from about 100 mg/kg to about 125 mg/kg, or from about 125 mg/kg to about 150 mg/kg of serabelisib. In some embodiments, a composition of the disclosure can be administered with less than about 30 mg/kg, about 50 mg/kg, about 75 mg/kg, about 100 mg/kg, about 125 mg/kg, or about 150 mg/kg of serabelisib. In some embodiments, a composition of the disclosure can be administered with less than about 30 mg/kg of serabelisib. In some embodiments, a composition of the disclosure can be administered with less than about 60 mg/kg of serabelisib. In some embodiments, a composition of the disclosure can be administered with less than about 100 mg/kg of serabelisib.

In some embodiments, the therapeutic agent is a PI3K inhibitor. In some embodiments, the PI3K inhibitor is serabelisib. In some embodiments, the therapeutically effective amount of serabelisib is about 30 mg/kg to about 150 mg/kg. In some embodiments, the therapeutically effective amount of serabelisib is less than about 60 mg/kg.

In some embodiments, a composition of the disclosure can be administered with bevacizumab. In some embodiments, a composition of the disclosure can be administered with bevacizumab in an amount of from about 1 mg/kg to about 2 mg/kg, from about 2 mg/kg to about 2 mg/kg, from about 3 mg/kg to about 4 mg/kg, from about 4 mg/kg to about 5 mg/kg, from about 5 mg/kg to about 6 mg/kg, or from about 6 mg/kg to about 7 mg/kg. In some embodiments, a composition of the disclosure can be administered with bevacizumab in an amount of from about 3 mg/kg to about 4 mg/kg. In some embodiments, a composition of the disclosure can be administered with bevacizumab in an amount of from about 4 mg/kg to about 5 mg/kg. In some embodiments, a composition of the disclosure can be administered with bevacizumab in an amount of from about 5 mg/kg to about 6 mg/kg.

In some embodiments, a composition of the disclosure can be administered with bevacizumab in an amount of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, or about 7 mg/kg. In some embodiments, a composition of the disclosure can be administered with bevacizumab in an amount of about 3 mg/kg. In some embodiments, a composition of the disclosure can be administered with bevacizumab in an amount of about 4 mg/kg. In some embodiments, a composition of the disclosure can be administered with bevacizumab in an amount of about 5 mg/kg. In some embodiments, a composition of the disclosure can be administered with bevacizumab in an amount of about 6 mg/kg.

In some embodiments, bevacizumab can be administered orally. In some embodiments, bevacizumab can be administered intravenously.

In some embodiments, the methods of the disclosure comprise administering a composition of the disclosure as a second line therapy, after administration of a first line therapy. In some embodiments, the first line therapy is administration of FOLFOX, wherein the FOLFOX comprises 5-fluorouracil, leucovorin, and oxaliplatin. In some embodiments, the methods of the disclosure comprise administering a composition of the disclosure as a second line therapy, after administration of FOLFOX4 as a first line therapy. In some embodiments, FOLFOX4 comprises administering: 1) on day 1:85 mg/m² IV oxaliplatin infusion in 250-500 mL D5W and 200 mg/m² of IV leucovorin in D5W administered concurrently over 120 minutes in separate bags using a Y-line, followed by 400 mg/m² IV 5-fluorouracil bolus given over 2-4 minutes, followed by 600 mg/m² IV 5-fluorouracil infusion in 500 mL D5W as a 22 hour continuous infusion; and 2) on day 2:200 mg/m² IV leucovorin infusion over 120 minutes followed by 400 mg/m² IV 5-fluorouracil bolus given over 2-4 minutes, followed by 600 mg/m² IV 5-fluorouracil infusion in 500 mL D5W as a 22 hour continuous infusion.

In some embodiments, the methods of the disclosure comprise administering a composition of the disclosure as a second line therapy, after administration of FOLFOX6 as a first line therapy. In some embodiments, FOFLOX6 comprises administering: 1) on days 1-2, oxaliplatin 100 mg/m² IV infusion, given as a 120 minutes IV infusion in 500 mL D5W, concurrent with leucovorin 400 mg/m² (or levoleucovorin 200 mg/m²) IV infusion, followed by 5-FU 400 mg/m² IV bolus, followed by 46-hour 5-FU infusion (2400 mg/m² for first two cycles, and may be increased to 3000 mg/m² if tolerated by patient (no toxicity>grade 1 during the first two cycles); and 2) resting on days 3-14.

In some embodiments, a composition of the disclosure can be administered with FOLFIRI, which comprises folinic acid or a pharmaceutically acceptable salt thereof, 5-fluorouracil or a pharmaceutically acceptable salt thereof, and irinotecan or a pharmaceutically acceptable salt thereof. In some embodiments, a composition of the disclosure can be administered with FOLFIRI comprising from about 100 mg/m² to about 250 mg/m² irinotecan; from about 100 mg/m² to about 300 mg/m² of leucovorin or a pharmaceutically acceptable salt thereof as a 2 h infusion during administration of irinotecan; immediately followed by a bolus dose of from about 300 mg/m² to about 500 mg/m² of 5-fluorouracil and a 46 h continuous infusion of from about 2000 mg/m² of to about 3000 mg/m² every 2 weeks. In some embodiments, a composition of the disclosure can be administered with FOLFIRI comprising 180 mg/m² irinotecan as a 90-min infusion on day 1; 200 mg/m² of leucovorin or a pharmaceutically acceptable salt thereof as a 2 h infusion during administration of irinotecan; immediately followed by a bolus dose of 400 mg/m² of 5-fluorouracil and a 46 h continuous infusion of 2400 mg/m² every 2 weeks.

In some embodiments, a composition of the disclosure can be administered with from about 100 mg/m² to about 250 mg/m² of irinotecan IV over 90 minutes on day 1; from about 300 mg/m² to about 500 mg/m² of leucovorin or a pharmaceutically acceptable salt thereof IV over 120 minutes concurrently with irinotecan on day 1; from about 300 mg/m² to about 500 mg/m² of 5-fluorouracil IV bolus after leucovorin or a pharmaceutically acceptable salt thereof non day 1; and from about 2000 mg/m² to about 3000 mg/m² of 5-fluorouracil IV continuous infusion start on day 1 over 46 hours; with or without from about 3 mg/kg to about 7 mg/kg of bevacizumab IV over 90 minutes for Day 1 initial dose. In some embodiments, a composition of the disclosure can be administered with 180 mg/m² of irinotecan IV over 90 minutes on day 1; 400 mg/m² of leucovorin or a pharmaceutically acceptable salt thereof IV over 120 minutes concurrently with irinotecan on day 1; 400 mg/m² of 5-fluorouracil IV bolus after leucovorin or a pharmaceutically acceptable salt thereof on day 1; and 2400 mg/m² of 5-fluorouracil IV continuous infusion start on day 1 over 46 hours; with or without 5 mg/kg bevacizumab IV over 90 minutes for Day 1 initial dose.

In some embodiments, the methods of the disclosure comprise administering a composition of the disclosure and FOLFIRINOX, with or without bevacizumab, as a second line therapy, after administration of a first line therapy.

### C) Immunotherapy

In some embodiments, the chemotherapeutic regimen is an immunotherapy. In some embodiments, the immunotherapy is an antibody therapy. In some embodiments, the antibody therapy is treatment with alemtuzumab, rituximab, ibritumomab tiuxetan, or ofatumumab. In some embodiments, the immunotherapy is an interferon. In some embodiments, the interferon is interferon α. In some embodiments, the immunotherapy is an interleukin, for example, IL-2. In some embodiments, the immunotherapy is an interleukin inhibitor, for example, an IRAK4 inhibitor.

In some embodiments, the immunotherapy is a cancer vaccine. In some embodiments, the cancer vaccine is a prophylactic vaccine. In some embodiments, the cancer vaccine is a treatment vaccine. In some embodiments, the cancer vaccine is an HPV vaccine, for example, Gardisil™, Cervarix, Oncophage, or Sipuleucel-T. In some embodiments, the immunotherapy is gp100. In some embodiments, the immunotherapy is a dendridic cell-based vaccine, for example, Ad.p53 DC. In some embodiments, the immunotherapy is a toll-like receptor modulator, for example, TLR-7 or TLR-9. In some embodiments, the immunotherapy is a PD-1, PD-L1, PD-L2, or CTL4-A modulator, for example, nivolumab. In some embodiments, the immunotherapy is an IDO inhibitor, for example, indoximod. In some embodiments, the immunotherapy is an anti-PD-1 monoclonal antibody, for example, MK3475 or nivolumab. In some embodiments, the immunotherapy is an anti-PD-L1 monoclonal antibody, for example, MEDI-4736 or RG-7446. In some embodiments, the immunotherapy is an anti-PD-L2 monoclonal antibody. In some embodiments, the immunotherapy is an anti-CTL1-4 antibody, for example, ipilumumab.

Cancer cells can change cellular metabolism to support elevated energetic and anabolic demands of proliferation of cancer cells. Examples of altered metabolism include aerobic glycolysis (i.e., Warburg effect) and high dependency on non-essential amino acids. One-carbon metabolism encompasses a collection of metabolic pathways that allow cells to generate and use molecules containing single carbons. One-carbon units (i.e., methyl groups) are carried and activated for use by tetrahydrofolates (THF), derived from dietary folate. Cells require one-carbon units to support nucleotide synthesis, methylation reactions and reductive metabolism. Cancer cells are dependent on the one-carbon pathways for supporting high proliferative rates, and one-carbon metabolism is crucial for cancer cell proliferation.

THF-dependent one-carbon metabolism is a critical metabolic process underpinning cellular proliferation supplying carbons for the synthesis of nucleotides incorporated into DNA and RNA. Tryptophan is a theoretical source of one-carbon units through metabolism by indoleamine 2,3-dioxygenase 1 (IDO1). In IDO1 expressing cancer cells, tryptophan is a bona fide one-carbon donor for purine nucleotide synthesis both in vitro and in vivo.

In cancer cell metabolism, serine is considered the predominant source of one-carbon units. Serine is obtained either by de novo synthesis from the glycolytic intermediate 3-phosphoglycerate via the serine synthesis pathway (SSP), or by uptake from the extracellular environment. Some cancer cells display increased SSP enzyme expression in order to meet cellular serine demands, whereas others rely predominantly on serine uptake. Serine hydro methyltransferases (SHMT1 and SHMT2) directly catalyze the conversion of serine into glycine and the release of a one-carbon, which enters the THE cycle.

The amino acids glycine, histidine and tryptophan are also potential one-carbon donors. Glycine can provide one-carbon units through the glycine cleavage system (GCS). Histidine catabolism can also yield one-carbon units and can further sensitize cancer cells to anti-folate treatment due to a decrease in free THF pools.

As an essential amino acid, tryptophan is critical for protein synthesis, but is also a precursor for 5-hydroxytryptamine and kynurenine production. In the kynurenine pathway, the initial and rate-limiting step is the conversion of tryptophan to formyl-kynurenine. Three enzymes are capable of catalyzing this reaction: IDO1, IDO2, and TDO. Both IDO2 and TDO have low expression levels and limited tissue specificity, and IDO1 is considered the predominant form. Formyl-kynurenine spontaneously forms kynurenine, with the release of a molecule of formate. Formate can enter the one-carbon cycle by directly reacting with THF and it is via this pathway that tryptophan can serve as a one-carbon donor.

IDO1 activity depletes tryptophan and increases kynurenine in the tumor microenvironment, causing a range of effects on immune cells. Tryptophan depletion decreases tumor infiltrating T-cell activity, and kynurenine decreases effector T-cell proliferation and supports the differentiation of immunosuppressive T-regulatory cells through binding of the aryl hydrocarbon receptor. The tumor micro-environmental effects provide an immunologically permissive environment for tumor growth. The kynurenine pathway has several metabolic outputs, including: reactive oxygen species (superoxide) levels, one-carbon metabolism, synthesis of NAD (P)+, synthesis of alanine and entry of carbons (via α-ketoadipate) into the TCA cycle.

Disclosed herein is a method of treating a cancer in a subject in need thereof, the method comprising a) administering to the subject a therapeutically-effective amount of a pharmaceutical composition, wherein the pharmaceutical composition is substantially devoid of at least two amino acids; and b) an IDO1 inhibitor. In some embodiments, the at least two amino acids is serine and glycine.

In some embodiments, the IDO1 inhibitor is indoximod (D-1MT; NLG-8189), 4-phenylimidazole (4-PI), N3-benzyl substituted 4-PI, ortho-hydroxy 4-PI, navoximod, or epacadostat. In some embodiments, the IDO1 inhibitor is epacadostat.

In some embodiments, a composition of the disclosure and an IDO1 inhibitor can be used to treat a cancer. In some embodiments, the cancer is pancreatic cancer. In some embodiments, the cancer is colon cancer. In some embodiments, the cancer is breast cancer. In some embodiments, the cancer is cervical cancer. In some embodiments, the cancer is lung cancer.

In some embodiments, the IDO1 inhibitor is administered 1, 2, 3, 4, or 5 times daily in combination with an amino acid modulation therapy. In some embodiments, the IDO1 inhibitor is administered once daily in combination with an amino acid modulation therapy. In some embodiments, the IDO1 inhibitor is administered twice daily in combination with an amino acid modulation therapy. In some embodiments, the IDO1 inhibitor is administered three times daily in combination with an amino acid modulation therapy.

In some embodiments, the IDO1 inhibitor is administered in an amount of from about 10 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, or about 450 mg to about 500 mg. In some embodiments, the IDO1 inhibitor is administered in an amount of from about 50 mg to about 100 mg. In some embodiments, the IDO1 inhibitor is administered in an amount of from about 100 mg to about 150 mg. In some embodiments, the IDO1 inhibitor is administered in an amount of from about 250 mg to about 300 mg.

In some embodiments, the IDO1 inhibitor is administered in an amount of about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In some embodiments, the IDO1 inhibitor is administered in an amount of about 25 mg. In some embodiments, the IDO1 inhibitor is administered in an amount of about 50 mg. In some embodiments, the IDO1 inhibitor is administered in an amount of about 100 mg. In some embodiments, the IDO1 inhibitor is administered in an amount of about 300 mg.

In some embodiments, about 25 mg of epacadostat is administered to a subject in combination with serine and glycine modulation therapy. In some embodiments, about 50 mg of epacadostat is administered to a subject in combination with serine and glycine modulation therapy. In some embodiments, about 100 mg of epacadostat is administered to a subject in combination with serine and glycine modulation therapy. In some embodiments, about 300 mg of epacadostat is administered to a subject in combination with serine and glycine modulation therapy.

### D) Other Therapeutic Agents

In some embodiments, a composition of the disclosure can be administered with a therapeutic agent that is not a chemotherapeutic or immunotherapeutic agent. In some embodiments, a composition of the disclosure is administered with disulfiram.

In some embodiments, a composition of the disclosure is administered with from about 125 mg to about 250 mg of disulfiram. In some embodiments, a composition of the disclosure is administered with from about 125 mg to about 150 mg, from about 150 mg to about 175 mg, from about 175 mg to about 200 mg, from about 200 mg to about 225 mg, or from about 225 mg to about 250 mg of disulfiram. In some embodiments, a composition of the disclosure is administered with less than about 125 mg, about 150 mg, about 175 mg, about 200 mg, about 225 mg, or about 250 mg of disulfiram. In some embodiments, a composition of the disclosure is administered with less than about 125 mg of disulfiram. In some embodiments, a composition of the disclosure is administered with less than about 150 mg of disulfiram. In some embodiments, a composition of the disclosure is administered with less than about 200 mg of disulfiram. In some embodiments, a composition of the disclosure is administered with less than about 250 mg of disulfiram.

In some embodiments, the therapeutically effective amount of disulfiram is about 125 mg to about 500 mg. In some embodiments, the therapeutically effective amount of disulfiram is less than about 250 mg.

### Cancer

In some embodiments, a cancer can comprise a malignant cell type, such as a solid tumor or a hematological tumor. In some embodiments, a cancer can comprise a tumor of an organ selected from the group consisting of pancreas, colon, cecum, stomach, gallbladder, skin, brain, head, neck, ovary, kidney, larynx, sarcoma, lung, bladder, melanoma, prostate, and breast. In some embodiments, a cancer can comprise hematological tumors include tumors of the bone marrow, T or B cell malignancies, leukemias, lymphomas, blastomas, myelomas, and the like. In some embodiments, a cancer can also comprise carcinoma, lymphoma, blastoma, sarcoma, leukemia, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer and gastrointestinal stromal cancer), pancreatic cancer, glioblastoma, cervical cancer, ovarian cancer, liver cancer, bladder cancer, gallbladder cancer, breast cancer, colon cancer, colorectal cancer, endometrial or uterine carcinoma, salivary gland carcinoma, kidney or renal cancer, renal cell carcinoma, prostate cancer, vulval cancer, thyroid cancer, various types of head and neck cancer, head and neck squamous cell carcinoma, melanoma, superficial spreading melanoma, lentigo malignant melanoma, acral lentiginous melanomas, nodular melanomas, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's macroglobulinemia), chronic lymphocytic leukemia (CLL), acute lymphoblastic leukemia (ALL), Hairy cell leukemia, multiple myeloma, acute myeloid leukemia (AML) and chronic myeloblastic leukemia.

In some embodiments, the cancer is carcinoma, undifferentiated carcinoma, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, adenocarcinoma, gastrinoma, malignant, cholangiocarcinoma, hepatocellular carcinoma, combined hepatocellular carcinoma and cholangiocarcinoma, trabecular adenocarcinoma, adenoid cystic carcinoma, adenocarcinoma in adenomatous polyp, adenocarcinoma, familial polyposis coli, solid carcinoma, carcinoid tumor, malignant, branchiolo-alveolar adenocarcinoma, papillary adenocarcinoma, chromophobe carcinoma, acidophil carcinoma, oxyphilic adenocarcinoma, basophil carcinoma, clear cell adenocarcinoma, granular cell carcinoma, follicular adenocarcinoma, papillary and follicular adenocarcinoma, nonencapsulating sclerosing carcinoma, adrenal cortical carcinoma, endometroid carcinoma, skin appendage carcinoma, apocrine adenocarcinoma, sebaceous adenocarcinoma, ceruminous adenocarcinoma, mucoepidermoid carcinoma, cystadenocarcinoma, papillary cystadenocarcinoma, papillary serous cystadenocarcinoma, mucinous cystadenocarcinoma, mucinous adenocarcinoma, signet ring cell carcinoma, infiltrating duct carcinoma, medullary carcinoma, lobular carcinoma, inflammatory carcinoma, paget's disease, mammary, acinar cell carcinoma, adenosquamous carcinoma, adenocarcinoma w/squamous metaplasia, thymoma, malignant, ovarian stromal tumor, malignant, thecoma, malignant, granulosa cell tumor, malignant, androblastoma, malignant, sertoli cell carcinoma, leydig cell tumor, malignant, lipid cell tumor, malignant, paraganglioma, malignant, extra-mammary paraganglioma, malignant, pheochromocytoma, glomangiosarcoma, malignant melanoma, amelanotic melanoma, superficial spreading melanoma, malignant melanoma in giant pigmented nevus, epithelioid cell melanoma, blue nevus, malignant, sarcoma, fibrosarcoma, fibrous histiocytoma, malignant, myxosarcoma, liposarcoma, leiomyosarcoma, rhabdomyosarcoma, embryonal rhabdomyosarcoma, alveolar rhabdomyosarcoma, stromal sarcoma, mixed tumor, malignant, mullerian mixed tumor, nephroblastoma, hepatoblastoma, carcinosarcoma, mesenchymoma, malignant, brenner tumor, malignant, phyllodes tumor, malignant, synovial sarcoma, mesothelioma, malignant, dysgerminoma, embryonal carcinoma, teratoma, malignant, struma ovarii, malignant, choriocarcinoma, mesonephroma, malignant, hemangiosarcoma, hemangioendothelioma, malignant, Kaposi's sarcoma, hemangiopericytoma, malignant, lymphangiosarcoma, osteosarcoma, juxtacortical osteosarcoma, chondrosarcoma, chondroblastoma, malignant, mesenchymal chondrosarcoma, giant cell tumor of bone, Ewing's sarcoma, odontogenic tumor, malignant, ameloblastic odontosarcoma, ameloblastoma, malignant, ameloblastic fibrosarcoma, pinealoma, malignant, chordoma, glioma, malignant, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, malignant, neurofibrosarcoma, neurilemmoma, malignant, granular cell tumor, malignant, malignant lymphoma, Hodgkin's disease, Hodgkin's, paragranuloma, malignant lymphoma, small lymphocytic, malignant lymphoma, large cell, diffuse, malignant lymphoma, follicular, mycosis fungoides, other specified non-Hodgkin's lymphomas, malignant histiocytosis, multiple myeloma, mast cell sarcoma, immunoproliferative small intestinal disease, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, myeloid sarcoma, or hairy cell leukemia.

### Therapeutic Effects

Disclosed herein is a method of treating a cancer, the method comprising administering to a subject in need thereof a therapeutically-effective amount of a composition of the disclosure. In some embodiments, a composition of the disclosure can decrease cell proliferation, decrease tumor size, or decrease an amount of at least one amino acid.

A composition of the disclosure can decrease cell proliferation in a subject. In some embodiments, administering a composition of a disclosure can decrease cell proliferation in a subject by from about 5% to about 10%, from about 10% to about 15%, from about 15% to about 20%, from about 20% to about 25%, from about 25% to about 30%, from about 35% to about 40%, from about 40% to about 45%, from about 45% to about 50%, from about 50% to about 75%, from about 75% to about 100%, from about 100% to about 125%, from about 125% to about 150%, from about 150% to about 175%, or from about 175% to about 200% compared to a subject that is not administered the dietary composition. In some embodiments, administering a composition of a disclosure can decrease cell proliferation in a subject by from about 20% to about 25% compared to a subject that is not administered the dietary composition. In some embodiments, administering a composition of a disclosure can decrease cell proliferation in a subject by from about 50% to about 75% compared to a subject that is not administered the dietary composition.

In some embodiments, administering a composition of a disclosure can decrease cell proliferation in a subject by about 5%, about 10%, about 15%, about 20%, about 25%, about 30%, about 35%, about 40%, about 45%, about 50%, about 75%, about 100%, about 125%, about 150%, about 175%, or about 200% compared to a subject that is not administered the dietary composition. In some embodiments, administering a composition of a disclosure can decrease cell proliferation in a subject by about 20% compared to a subject that is not administered the dietary composition. In some embodiments, administering a composition of a disclosure can decrease cell proliferation in a subject by about 30% compared to a subject that is not administered the dietary composition. In some embodiments, administering a composition of a disclosure can decrease cell proliferation in a subject by about 50% compared to a subject that is not administered the dietary composition. In some embodiments, administering a composition of a disclosure can decrease cell proliferation in a subject by about 70% compared to a subject that is not administered the dietary composition.

In some embodiments, administering a composition of the disclosure can decrease a tumor size in a subject by from about 5% to about 10%, from about 10% to about 15%, from about 15% to about 20%, from about 20% to about 25%, from about 25% to about 30%, from about 35% to about 40%, from about 40% to about 45%, from about 45% to about 50%, from about 50% to about 75%, from about 75% to about 100%, from about 100% to about 125%, from about 125% to about 150%, from about 150% to about 175%, or from about 175% to about 200%. In some embodiments, administering a composition of the disclosure can decrease a tumor size in a subject by from about 20% to about 25%. In some embodiments, administering a composition of the disclosure can decrease a tumor size in a subject by from about 45% to about 50%.

In some embodiments, administering a composition of the disclosure can decrease a tumor size in a subject by about 5%, about 10%, about 15%, about 20%, about 25%, about 30%, about 35%, about 40%, about 45%, about 50%, about 75%, about 100%, about 125%, about 150%, about 175%, or about 200%. In some embodiments, administering a composition of the disclosure can decrease a tumor size in a subject by about 20%. In some embodiments, administering a composition of the disclosure can decrease a tumor size in a subject by about 30%. In some embodiments, administering a composition of the disclosure can decrease a tumor size in a subject by about 50%.

In some embodiments, administering a composition of the disclosure devoid of at least proline, serine, or glycine can decrease an amount of circulating proline, serine, or glycine in the serum, plasma, or blood of a subject by at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, or at least about 98%. In some embodiments, administering a composition of the disclosure can decrease an amount of circulating proline, serine, or glycine in the serum, plasma, or blood of a subject by at least about 75%. In some embodiments, administering a composition of the disclosure can decrease an amount of circulating proline, serine, or glycine in the serum, plasma, or blood of a subject by at least about 80%. In some embodiments, administering a composition of the disclosure can decrease an amount of circulating proline, serine, or glycine in the serum, plasma, or blood of a subject by at least about 85%.

In some embodiments, administering a composition of the disclosure devoid of at least proline, serine, or glycine can decrease a level of tissue proline, serine, or glycine in the subject by at least about 10%, at least about 20%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, or at least about 98%, as measured by LC-MS analysis of a tissue sample. In some embodiments, administering a composition of the disclosure can decrease a level of tissue proline, serine, or glycine in the subject by at least about 75%. In some embodiments, administering a composition of the disclosure can decrease a level of tissue proline, serine, or glycine in the subject by at least about 80%. In some embodiments, administering a composition of the disclosure can decrease a level of tissue proline, serine, or glycine in the subject by at least about 85%.

In some embodiments, administering a composition of the disclosure devoid of at least proline, serine, or glycine and at least one additional amino acid can decrease an amount of circulating proline, serine, or glycine or the at least one additional amino acid in the subject by at least about 10%, at least about 20%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, or at least about 98%, as measured by LC-MS analysis of a tissue sample. In some embodiments, administering a composition of the disclosure can decrease an amount of circulating proline, serine, or glycine or the at least one additional amino acid in the subject by at least about 75%. In some embodiments, administering a composition of the disclosure can decrease an amount of circulating proline, serine, or glycine or the at least one additional amino acid in the subject by at least about 80%. In some embodiments, administering a composition of the disclosure can decrease an amount of circulating proline, serine, or glycine or the at least one additional amino acid in the subject by at least about 85%.

In some embodiments, administering a composition of the disclosure devoid of at least proline, serine, or glycine and at least one additional amino acid can decrease a level of tissue proline, serine, or glycine or the at least one additional amino acid in the subject by at least about 10%, at least about 20%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, or at least about 98%, as measured by LC-MS analysis of a tissue sample. In some embodiments, administering a composition of the disclosure can decrease a level of tissue proline, serine, or glycine or the at least one additional amino acid in the subject by at least about 75%. In some embodiments, administering a composition of the disclosure can decrease a level of tissue proline, serine, or glycine or the at least one additional amino acid in the subject by at least about 80%. In some embodiments, administering a composition of the disclosure can decrease a level of tissue proline, serine, or glycine or the at least one additional amino acid in the subject by at least about 85%.

In some embodiments, administering a composition of the disclosure devoid of at least proline, serine, or glycine can decrease an amount of circulating proline, serine, or glycine in the subject for at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, at least about 22 hours, at least about 24 hours, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days, as measured by LC-MS analysis of a tissue sample. In some embodiments, administering a composition of the disclosure devoid of at least proline, serine, or glycine can decrease an amount of circulating proline, serine, or glycine in the subject for at least about 8 hours. In some embodiments, administering a composition of the disclosure devoid of at least proline, serine, or glycine can decrease an amount of circulating proline, serine, or glycine in the subject for at least about 12 hours. In some embodiments, administering a composition of the disclosure devoid of at least proline, serine, or glycine can decrease an amount of circulating proline, serine, or glycine in the subject for at least about 18 hours.

In some embodiments, administering a composition of the disclosure devoid of at least proline, serine, or glycine can decrease a level of tissue proline, serine, or glycine in the subject for at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, at least about 22 hours, at least about 24 hours, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, or at least about 14 days, as measured by LC-MS analysis of a tissue sample. In some embodiments, administering a composition of the disclosure devoid of at least proline, serine, or glycine can decrease a level of tissue proline, serine, or glycine in the subject for at least about 8 hours. In some embodiments, administering a composition of the disclosure devoid of at least proline, serine, or glycine can decrease a level of tissue proline, serine, or glycine in the subject for at least about 12 hours. In some embodiments, administering a composition of the disclosure devoid of at least proline, serine, or glycine can decrease a level of tissue proline, serine, or glycine in the subject for at least about 18 hours.

Administering a composition of the disclosure devoid of at least proline, serine, or glycine can increase overall survival of a subject. In some embodiments, administering a composition of the disclosure devoid of at least proline, serine, or glycine can increase overall survival of a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, administering a composition of the disclosure devoid of at least proline, serine, or glycine can increase overall survival of a subject by at least about 10%. In some embodiments, administering a composition of the disclosure devoid of at least proline, serine, or glycine can increase overall survival of a subject by at least about 20%. In some embodiments, administering a composition of the disclosure devoid of at least proline, serine, or glycine can increase overall survival of a subject by at least about 30%.

Administering a composition of the disclosure devoid of at least proline, serine, or glycine can increase progression free survival of a subject. In some embodiments, administering a composition of the disclosure devoid of at least proline, serine, or glycine can increase progression free survival of a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, administering a composition of the disclosure devoid of at least proline, serine, or glycine can increase progression free survival of a subject by at least about 10%. In some embodiments, administering a composition of the disclosure devoid of at least proline, serine, or glycine can increase progression free survival of a subject by at least about 20%. In some embodiments, administering a composition of the disclosure devoid of at least proline, serine, or glycine can increase progression free survival of a subject by at least about 30%.

Administering a composition of the disclosure devoid of at least proline, serine, or glycine can increase percentage of cancer cell death. In some embodiments, administering a composition of the disclosure devoid of at least proline, serine, or glycine can increase percentage of cancer cell death by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, administering a composition of the disclosure devoid of at least proline, serine, or glycine can increase percentage of cancer cell death by at least about 10%. In some embodiments, administering a composition of the disclosure devoid of at least proline, serine, or glycine can increase percentage of cancer cell death by at least about 20%. In some embodiments, administering a composition of the disclosure devoid of at least proline, serine, or glycine can increase percentage of cancer cell death by at least about 30%.

Administering a composition of the disclosure devoid of at least proline, serine, or glycine can increase ferroptosis in a tumor cell. In some embodiments, administering a composition of the disclosure devoid of at least proline, serine, or glycine can increase ferroptosis in a tumor cell by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, administering a composition of the disclosure devoid of at least proline, serine, or glycine can increase ferroptosis in a tumor cell by at least about 10%. In some embodiments, administering a composition of the disclosure devoid of at least proline, serine, or glycine can increase ferroptosis in a tumor cell by at least about 20%. In some embodiments, administering a composition of the disclosure devoid of at least proline, serine, or glycine can increase ferroptosis in a tumor cell by at least about 30%.

Administering a composition of the disclosure devoid of at least proline, serine, or glycine can increase sensitivity to a cancer therapy in a subject. In some embodiments, administering a composition of the disclosure devoid of at least proline, serine, or glycine can increase sensitivity to a cancer therapy in a subject by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, administering a composition of the disclosure devoid of at least proline, serine, or glycine can increase sensitivity to a cancer therapy in a subject by at least about 10%. In some embodiments, administering a composition of the disclosure devoid of at least proline, serine, or glycine can increase sensitivity to a cancer therapy in a subject by at least about 20%. In some embodiments, administering a composition of the disclosure devoid of at least proline, serine, or glycine can increase sensitivity to a cancer therapy in a subject by at least about 30%.

Administering a composition of the disclosure devoid of at least proline, serine, or glycine can increase a treatment response rate of a therapeutic agent. In some embodiments, administering a composition of the disclosure devoid of at least proline, serine, or glycine can increase a treatment response rate of a therapeutic agent by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, administering a composition of the disclosure devoid of at least proline, serine, or glycine can increase a treatment response rate of a therapeutic agent by at least about 10%. In some embodiments, administering a composition of the disclosure devoid of at least proline, serine, or glycine can increase a treatment response rate of a therapeutic agent by at least about 20%. In some embodiments, administering a composition of the disclosure devoid of at least proline, serine, or glycine can increase a treatment response rate of a therapeutic agent by at least about 30%.

Administering a composition of the disclosure devoid of at least proline, serine, or glycine with a therapeutic agent can increase the efficacy of the therapeutic agent in a subject compared to a subject treated only with the composition of the disclosure. In some embodiments, administering a composition of the disclosure devoid of at least proline, serine, or glycine with a therapeutic agent can increase the efficacy of the therapeutic agent in a subject by at least about at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% compared to a subject treated only with the composition of the disclosure. In some embodiments, administering a composition of the disclosure devoid of at least proline, serine, or glycine with a therapeutic agent can increase the efficacy of the therapeutic agent in a subject by at least about at least about 10% compared to a subject treated only with the composition of the disclosure. In some embodiments, administering a composition of the disclosure devoid of at least proline, serine, or glycine with a therapeutic agent can increase the efficacy of the therapeutic agent in a subject by at least about at least about 20% compared to a subject treated only with the composition of the disclosure. In some embodiments, administering a composition of the disclosure devoid of at least proline, serine, or glycine with a therapeutic agent can increase the efficacy of the therapeutic agent in a subject by at least about at least about 30% compared to a subject treated only with the composition of the disclosure.

Administering a composition of the disclosure devoid of at least proline, serine, or glycine with a therapeutic agent to a subject can have a decreased dose of the therapeutic agent compared to a subject treated with the therapeutic agent alone to achieve the same outcome. In some embodiments, administering a composition of the disclosure devoid of at least proline, serine, or glycine with a therapeutic agent to a subject can decrease the required dose of the therapeutic agent by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% compared to a subject treated with the therapeutic agent alone to achieve the same outcome. In some embodiments, administering a composition of the disclosure devoid of at least proline, serine, or glycine with a therapeutic agent to a subject can decrease the required dose of the therapeutic agent by at least about 10% compared to a subject treated with the therapeutic agent alone to achieve the same outcome. In some embodiments, administering a composition of the disclosure devoid of at least proline, serine, or glycine with a therapeutic agent to a subject can decrease the required dose of the therapeutic agent by at least about 20% compared to a subject treated with the therapeutic agent alone to achieve the same outcome. In some embodiments, administering a composition of the disclosure devoid of at least proline, serine, or glycine with a therapeutic agent to a subject can decrease the required dose of the therapeutic agent by at least about 30% compared to a subject treated with the therapeutic agent alone to achieve the same outcome.

Administering a composition of the disclosure devoid of at least proline, serine, or glycine with a therapeutic agent to a subject can decrease adverse events associated with an additional therapy compared to a subject treated with the additional therapy alone. In some embodiments, administering a composition of the disclosure devoid of at least proline, serine, or glycine with a therapeutic agent to a subject can decrease adverse events associated with an additional therapy by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% compared to a subject treated with the additional therapy alone. In some embodiments, administering a composition of the disclosure devoid of at least proline, serine, or glycine with a therapeutic agent to a subject can decrease adverse events associated with an additional therapy by at least about 10% compared to a subject treated with the additional therapy alone. In some embodiments, administering a composition of the disclosure devoid of at least proline, serine, or glycine with a therapeutic agent to a subject can decrease adverse events associated with an additional therapy by at least about 20% compared to a subject treated with the additional therapy alone. In some embodiments, administering a composition of the disclosure devoid of at least proline, serine, or glycine with a therapeutic agent to a subject can decrease adverse events associated with an additional therapy by at least about 30% compared to a subject treated with the additional therapy alone.

In some embodiments, an amount of a biosynthetic metabolic enzyme can be quantified by comparing the amount of the biosynthetic metabolic enzyme from one biological sample to an amount of the biosynthetic metabolic enzyme from a reference sample, wherein the reference sample does not comprise a cancer cell.

In some embodiments, an amount of a biosynthetic metabolic enzyme can be quantified using an empirical scoring system. In some embodiments, a quantification result can be evaluated by a semiquantitative approach. In some embodiments, immunohistochemistry results can be assigned an H-score (or “histo” score) to tumor samples. First, membrane staining intensity (0, 1+, 2+, or 3+) can be determined for each cell in a fixed field. The cells can be categorized as negative (0), weak (1+), moderate (2+), and strongly (3+) stained membranes. The H-score can be based on a predominant staining intensity and include the sum of individual H-scores for each intensity level seen. In some embodiments, the percentage of cells at each staining intensity level can be calculated, and an H-score can be assigned using the following formula:

$$[ 1 \times ( \% cells 1 + ) + 2 \times ( \% cells 2 + ) + 3 \times ( \% cells 3 + ) ]$$

The final score, ranging from 0 to 300, gives more relative weight to higher-intensity membrane staining in a given tumor sample. The sample can then be considered positive or negative on the basis of a specific discriminatory threshold.

### EXAMPLES

### Example 1: Stratification of Patients to Receive a Diet Lacking Serine and Glycine

Sample preparation: A tumor biopsy sample (e.g., fresh or archival formalin-fixed paraffin-embedded (FFPE) tissue) is obtained from a patient. Briefly, the sample is cut into tissue sections with a microtome and mounted on slides. The sample slide is de-waxed and subjected to an antigen retrieval method. The sample slide is then subjected to immunohistochemistry (IHC) staining with primary antibodies for PHGDH and PSAT1. Secondary antibodies conjugated to peroxidase enzymes are used to detect the primary antibodies. A substrate agent is added to label the primary-secondary antibody conjugates. The sample slide is counterstained with generic tissue stain to label nuclei. The sample slide with the tumor section is visualized using a light microscope. High-quality digital images are taken using a camera or a slide scanner.

### PSAT1 IHC in FFPE Human Tissue

Antibody: PSAT1
Clone/Species: n/a/Ms mAb
Vendor/Catalog #: DSHB/CPTC-PSAT1-2-S
Lot: Sep. 26, 2016
Vial Concentration: 36 μg/mL
Approved Ab Concentration: 0.2 μg/mL
Approved Ab Titer: 1:180
Instrument: Dako
Tissue Tested: Human

PSAT1 staining: To stain for PSAT1, the FFPE tissue is cut into 5 micron sections and placed on a positively charged slide. The sections on the slides are dried at 60° C. for 60 minutes. EnVision FLEX High pH target retrieval (50×) is diluted to a volume of 1500 mL. The diluted retrieval is placed in the Dako PT Link module and preheated to 65° C. The slides are then loaded onto the racks. The loaded racks are placed into the Dako PT Link module. Deparaffinization, rehydration, and antigen retrieval are performed in a Dako FLEX TRS High pH target retrieval at 97° C. for 30 minutes. After Dako PT Link completes the antigen retrieval process, the racks are removed from the module and left in Envision FLEX Wash Buffer (20×) for 5 minutes. The racks with the slides are then loaded Dako AutostainerLink 48 and stained with IHC using the FLEX/HRP visualization protocol. The slides are rinsed with 300 μL of EnVision FLEX Peroxidase-Blocking agent, incubated for 5 minutes at room temperature, and washed with Wash Buffer.

300 μL mouse monoclonal primary antibody pSAT1 is diluted to 0.2 μg/mL (1:180) in EnVision FLEX antibody diluent to 0.2 μg/mL (1:180) in EnVision FLEX antibody diluent. The slides are incubated with the diluted antibody for 20 minutes at room temperature, washed with Wash Buffer, incubated with 300 μL secondary reagent En Vision FLEX+Mouse (Linker) for 15 minutes at room temperature, and washed again in Wash Buffer. After staining with the antibodies, the slides are incubated with 300 μL labelled polymer EnVision FLEX/HRP for 20 minutes at room temperature and washed two times with Wash Buffer. On the second wash, the slides are incubated with the buffer for 5 minutes. 600 μL FLEX DAB+Sub-Chromogen, prepared offline at a dilution of 1 drop of DAB+Chromogen for each mL of DAB+Substrate Buffer (K8000/SM803), is applied to the slide. The slide is then incubated for 10 minutes. The slides are washed once in Wash Buffer and once with distilled water. After washing, the slides are unloaded, counterstained, and dehydrated on Leica Autostainer through graded series of histological grade alcohols to xylene and mounted with a slide cover-slipper.

### PHGDH IHC in FFPE Human Tissue

Antibody: PHGDH
Clone/Species: CL0555/Ms mAb
Vendor/Catalog #: Invitrogen/MA5-31357
Lot: WB3197262B
Vial Concentration: 1 mg/mL
Approved Ab Concentration: 0.2 μg/mL
Approved Ab Titer: 1:5000
Instrument: Dako
Tissue Tested: Human

PHGDH Staining: To stain for PHGDH, the FFPE tissue is cut into 5 micron sections and placed on a positively charged slide. The sections on the slides are dried at 60° C. for 60 minutes. EnVision FLEX Low pH target retrieval (50×) is diluted to a volume of 1500 mL. The diluted retrieval is placed in the Dako PT Link module and preheated to a temperature of 65° C. The slides are then loaded onto the racks. The loaded racks are placed into the module. Deparaffinization, rehydration, and antigen retrieval are performed in Dako FLEX TRS Low pH target retrieval at 97° C. for 30 minutes. After the Dako PT Link completes the antigen retrieval process, the racks are removed from module and placed in Envision FLEX Wash Buffer (20×) for 5 minutes. The racks with the slides are then loaded Dako AutostainerLink 48 and stained with IHC using the FLEX/HRP visualization protocol. The slides are rinsed with 300 μL of EnVision FLEX Peroxidase-Blocking, incubated for 5 minutes at room temperature, and washed with Wash Buffer.

300 μL rabbit monoclonal primary antibody PHGDH is diluted to 3 μg/ml (1:2147) in En Vision FLEX antibody diluent. The slides are incubated with the diluted antibody for 20 minutes at room temperature and washed twice with Wash Buffer. After staining with antibodies, the slides are incubated with 300 μL labelled polymer En Vision FLEX/HRP for 20 minutes at room temperature and washed twice with Wash Buffer. On the second wash, the slides are incubated with the buffer for 5 minutes. 600 μL FLEX DAB+Sub-Chromogen, prepared offline at a dilution of 1 drop of DAB+Chromogen for each mL of DAB+Substrate Buffer, is applied to and incubated with the slide for 10 minutes. The slides are washed once with Wash Buffer and once with distilled water. After washing, the slides are unloaded, counterstained, and dehydrated on Leica Autostainer through graded series of histological grade alcohols to xylene and mounted with a slide cover-slipper.

Samples of healthy tissue (e.g., tissue with matching cell, tissue, organ, or cancer state) are also examined as described above. Samples showing strong positive and negative staining for the relevant enzymes are used as positive and negative controls, respectively. The positive and negative controls are used to compare the tumor sample and to develop an effective scoring system.

For image analysis, the levels of staining of the enzymes are quantified by pathologist or qualified technician and are also quantified using computer software. Scoring is carried out for cancer cells and non-cancer (i.e. tumor stroma) cells. The level of each enzyme is scored as follows: (3+)=high level of staining, (2+)=moderate level of staining, (1+)=low level of staining, (−)=negative staining (e.g., the same as the background or a negative control). A single enzyme and a combined enzyme score system are generated, which provides a quantitative evaluation of the likelihood that the tumor will respond to a serine and glycine restricted diet. A tumor with at least one (−) score is predicted to be a likely positive responder, and this patient will receive the serine and glycine restricted diet. Subjects with tumor tissue score of 0 or 1+ for PHGDH or PSAT1 will be allowed to enroll. Subjects with a combined PHGDH and PSAT1 score of 3+ (e.g., PHGDH=2+, PSAT1=1+) will be allowed to enroll. A patient with all enzymes 3+/2+ is predicted to be a likely non-responder/negative responder and will not receive the serine and glycine restricted diet.

### Example 2: Stratification of Patients to Receive a Diet Lacking Proline

Prior to receiving a diet formulated to be deficient in proline, a tumor biopsy sample (e.g., fresh or archival FFPE tissue) is obtained from a patient. The sample is cut into tissue sections with a microtome and mounted on slides. The sample slide is de-waxed and subjected to an antigen retrieval method. The sample slide is then subjected to immuno-staining with primary antibodies for ALDH18A1, PYCR1, PYCR2, PYCR3, and PRODH. Relevant secondary antibodies conjugated to peroxidase enzymes are used to detect the primary antibodies. The relevant substrate agent is added to label the primary-secondary antibody conjugates. The sample slide is counterstained with generic tissue stain to label nuclei. The sample slide with the tumor section is visualized using a light microscope. High-quality digital images are taken using a camera or a slide scanner.

Samples of healthy tissue (e.g., tissue with matching cell, tissue, organ, or cancer state) are also examined as described above. Samples showing strong positive and negative staining for the relevant enzymes are used as positive and negative controls, respectively. The positive and negative controls are used to compare the tumor sample and to develop an effective scoring system.

For image analysis, the levels of staining of the enzymes are quantified by pathologist or qualified technician and are also quantified by computer software. Scoring is carried out for cancer cells and non-cancer (i.e. tumor stroma) cells. The level of each enzyme is scored as follows: (3+)=high level of staining, (2+)=moderate level of staining, (1+)=low level of staining, (−)=negative staining (e.g., the same as the background or a negative control). A single enzyme and a combined enzyme score system are generated. This score provides a quantitative evaluation of the likelihood that the tumor will respond to a proline restricted diet. A tumor with at least one (−) score is predicted to be a likely positive responder, and this patient will receive the proline restricted diet. Subjects with tumor tissue score of 0 or 1+ for any one of ALDH18A1, PYCR1, PYCR2, PYCR3, and PRODH will be allowed to enroll. A patient with all enzymes 3+/2+ is predicted to be a likely non-responder/negative responder and will not receive the proline restricted diet.

### Example 3: Biological Sample Analysis Using Empirical Scoring System

Immunohistochemistry results are evaluated by a semiquantitative approach used to assign an H-score (or “histo” score) to tumor samples. First, membrane staining intensity (0, 1+, 2+, or 3+) is determined for each cell in a fixed field. The cells are categorized as negative (0), weak (1+), moderate (2+), and strongly (3+) stained membranes. The H-score is based on a predominant staining intensity and includes the sum of individual H-scores for each intensity level seen. By one method, the percentage of cells at each staining intensity level is calculated, and an H-score is assigned using the following formula:

$$[ 1 \times ( \% cells 1 + ) + 2 \times ( \% cells 2 + ) + 3 \times ( \% cells 3 + ) ]$$

The final score, ranging from 0 to 300, gives more relative weight to higher-intensity membrane staining in a given tumor sample. The sample can then be considered positive or negative on the basis of a specific discriminatory threshold.

A patient FFPE sample block is sectioned at 4-5 micron thickness onto charged slides. IHC staining is carried out using a PHGDH and PSAT1 IHC assay. Stains are scored by a pathologist using manual microscopy. All controls are reviewed and show appropriate positive and negative reactivity. The patient is considered eligible for enrollment in therapy if the H-score for either PHGDH or PSAT1 is less than or equal to 120. TABLE 1 shows an example H-scoring of a patient tumor sample, which is eligible for enrollment in therapy because the H-scores for PHGDH and PSAT1 were less than the threshold of 120.

TABLE 1

| | | | | | H-score |
|---|---|---|---|---|---|
| Expression Staining | | | | | (1*1+) + (2*2+) + |
| Intensity | 0 | 1+ | 2+ | 3+ | (3*3+) |
| PHGDH -% of cells stained | 90 | 3 | 7 | 0 | 17 |
| PSAT1 -% of cells stained | 100 | 0 | 0 | 0 | 0 |
| Patient eligible for enrollment | — | — | — | — | Yes |

### Example 4: Combined PHGDH and PSAT1 Enzyme Expression Correlates with Cancer Cell Growth in Proline, Serine, and Glycine-Free Media

Human Pancreatic cancer cell lines (ASPC1, BXPC3, CFPAC1, DANG, PANC1, PATU8902) were seeded in 48-well plates and in complete media+10% FBS. 24 hours later, cells were washed with PBS, followed by addition of amino acid deficient media+10% dialyzed FBS. Cells were grown for 3 more days and cell growth was measured using Crystal Violet. The relative growth in PSG free media was plotted against the mRNA expression of the indicated enzymes. mRNA expression values were obtained from publicly available data online.

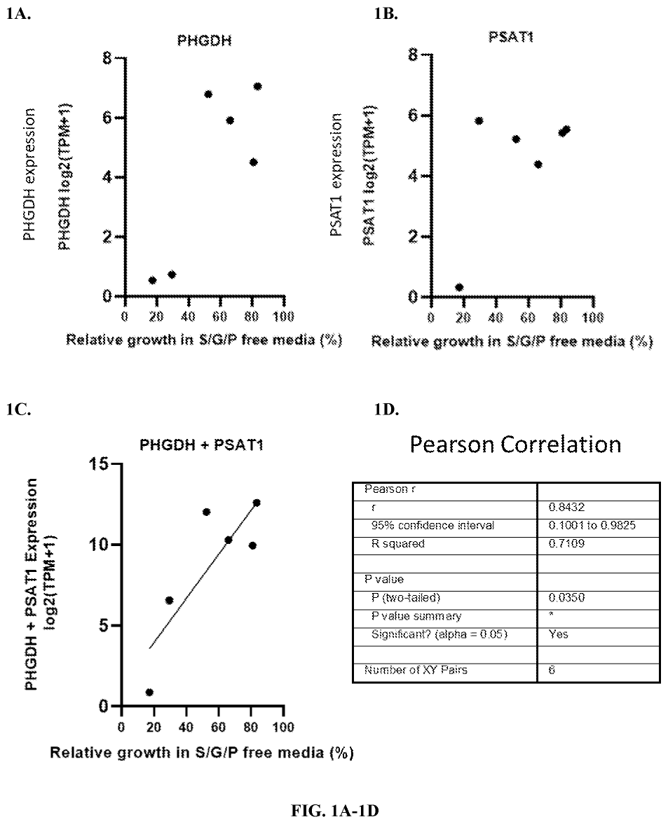

FIG. 1A shows the correlation between PHGDH enzyme expression and relative growth of cancer cells in serine, glycine, and proline-free media. FIG. 1B shows the correlation between PSAT1 enzyme expression and relative growth of cancer cells in serine, glycine, and proline-free media. FIG. 1C shows the correlation between PHGDH and PSAT 1 enzyme expression and relative growth of cancer cells in serine, glycine, and proline-free media. FIG. 1D shows Pearson Correlation values for combined PHGDH and PSAT1 enzyme expression with cancer cells growth in proline, serine, and proline-free media. The Pearson ‘r’ value of 0.8432 showed a high degree of correlation between the sum of PHGDH and PSAT1 expression and the response of the different cancer cells to serine, glycine, and proline restriction. Assessing PHGDH and PSAT1 expression was therefore an appropriate means to predict the likely sensitivity of cancer cells to serine, glycine, and proline restriction therapy.

### Example 5: Combined PHGDH, PSAT1, and PSPH Enzyme Expression Correlates with Cancer Cell Growth in Proline, Serine, and Glycine-Free Media

Human Pancreatic cancer cell lines (ASPC1, BXPC3, CFPAC1, DANG, PANC1, PATU8902) were seeded in 48-well plates, in complete media+10% FBS. 24 hours later, cells were washed with PBS, followed by addition of amino acid deficient media+10% dialysed FBS. Cells were grown for 3 more days and cell growth was measured using Crystal Violet. The relative growth in PSG free media was plotted against the mRNA expression of the indicated enzymes. mRNA expression values were obtained from publicly available data online.

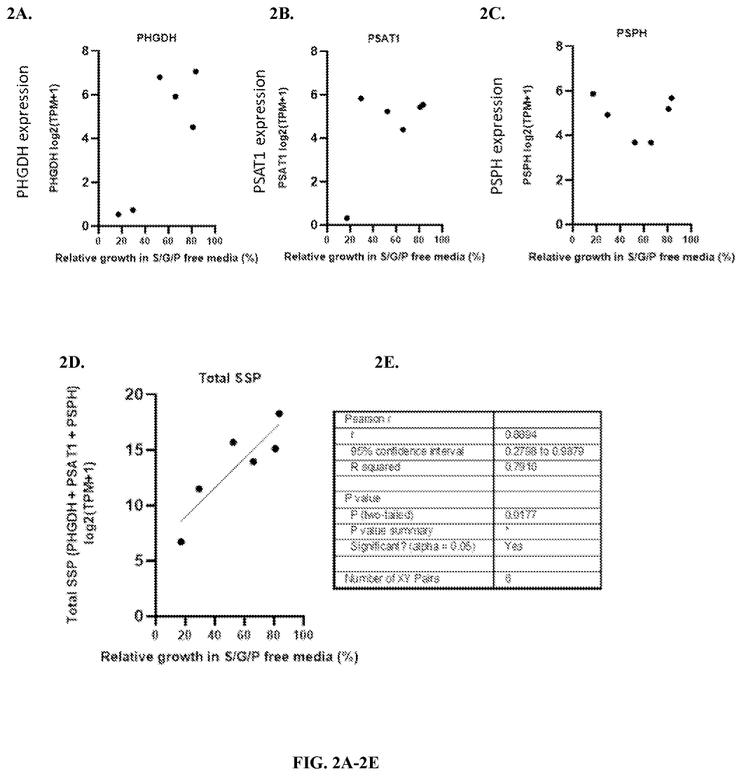

FIG. 2A shows the correlation between PHGDH enzyme expression and relative growth of cancer cells in serine, glycine, and proline-free media. FIG. 2B shows the correlation between PSAT1 enzyme expression and relative growth of cancer cells in serine, glycine, and proline-free media. FIG. 2C shows the correlation between PSPH enzyme expression and relative growth of cancer cells in serine, glycine, and proline-free media. FIG. 2D shows the correlation between PHGDH, PSAT1, and PSPH enzyme expression and relative growth of cancer cells in serine, glycine, and proline-free media. FIG. 2E shows Pearson Correlation values for combined PHGDH, PSAT1, and PSPH enzyme expression with cancer cells growth in proline, serine, and proline-free media. The Pearson ‘r’ value of 0.8894 showed a high degree of correlation between the sum of PHGDH, PSAT1 and PSPH expression and the response of the different cancer cells to serine, glycine and proline restriction. Assessing PHGDH, PSAT1, and PSPH expression was therefore an appropriate means to predict the likely sensitivity of cancer cells to serine, glycine, and proline restriction therapy.

### Example 6: Combined SHMT1 and SHMT2 Enzyme Expression does not Correlate with Cancer Cell Growth in Proline, Serine, and Glycine-Free Media

Human Pancreatic cancer cell lines (ASPC1, BXPC3, CFPAC1, DANG, PANC1, PATU8902) were seeded in 48-well plates, in complete media+10% FBS. 24 hours later, cells were washed with PBS, followed by addition of amino acid deficient media+10% dialysed FBS. Cells were grown for 3 more days and cell growth was measured using Crystal Violet. The relative growth in PSG free media was plotted against the mRNA expression of the indicated enzymes. mRNA expression values were obtained from publicly available data online.

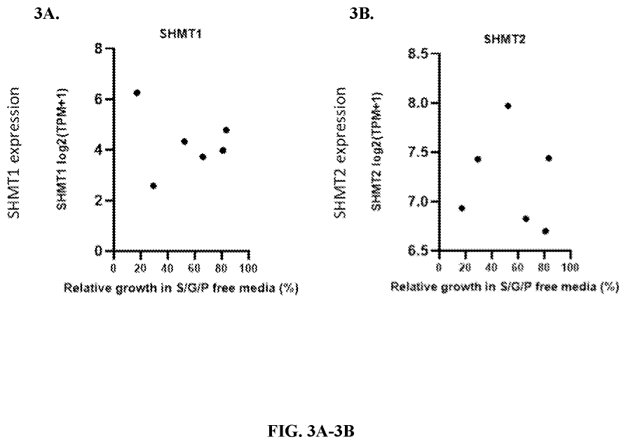

FIG. 3A shows the correlation between SHMT1 enzyme expression and relative growth of cancer cells in serine, glycine, and proline-free media. FIG. 3B shows the correlation between SHMT2 enzyme expression and relative growth of cancer cells in serine, glycine, and proline-free media. SHMT1 and SHMT2 are metabolic enzymes which interconvert serine and glycine, but SHMT1 and SHMT2 expression did not correlate with sensitivity of cancer cell lines to serine, glycine and proline restriction. The use of PHGDH and PSAT1 was a much better predictive test.

### Example 7: Study to Evaluate a Composition Disclosed Herein for Treatment of Cancer in a Subject in Combination with a Chemotherapy Drug

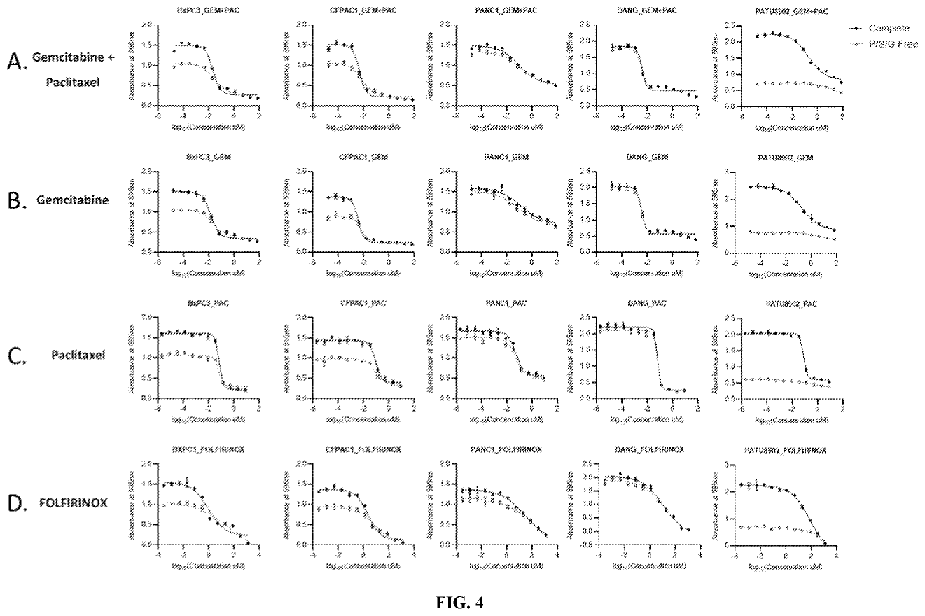

A study was performed to evaluate the efficacy of a disclosed composition in the treatment of cancer when combined with a chemotherapy drug. Pancreatic cancer cells such as BxPC3, CFPAC1, PANC1, DANG, and PATU8902 were treated with either Gemcitabine (GEM), Paclitaxel (PAC), GEM+PAC, and FOLFIRINOX (which is a combination of 5FU, Oxaliplatin, Folinic acid, and Irinotecan), in combination with complete media, or with media free of the amino acids proline/serine/glycine (P/S/G), as indicated in FIG. 4 rows A-D. Cells were seeded in 96-well plates and in complete media+10% FBS. 24 hours later, cells were washed with PBS followed by addition of the indicated drugs with either complete or P/S/G free media+10% dialyzed FBS. Cells were grown for 3 more days, and cell growth was measured using Crystal Violet staining. FIG. 4 illustrates that restriction of proline, serine, and glycine sensitized cancer cells to established chemotherapy drugs (row A: gemcitabine+paclitaxel; row B: gemcitabine; row C: paclitaxel; row D: FOLFIRINOX). Error bars denote the standard deviation. P/S/G starvation pushed the response curve down and to the left meaning lower concentrations of drug achieved the same cell killing activity as higher concentrations when proline, serine, and glycine were present. The data indicated that pancreatic cells (e.g., BxPC3, CPAC1, PANC1, DANG, and PATU8902) were more reactive to the drugs or drug combinations when subjected to proline/serine/glycine starvation conditions.

### Example 8: Study to Evaluate Compositions Disclosed Herein for Treatment of Cancer in Combination with Disulfiram

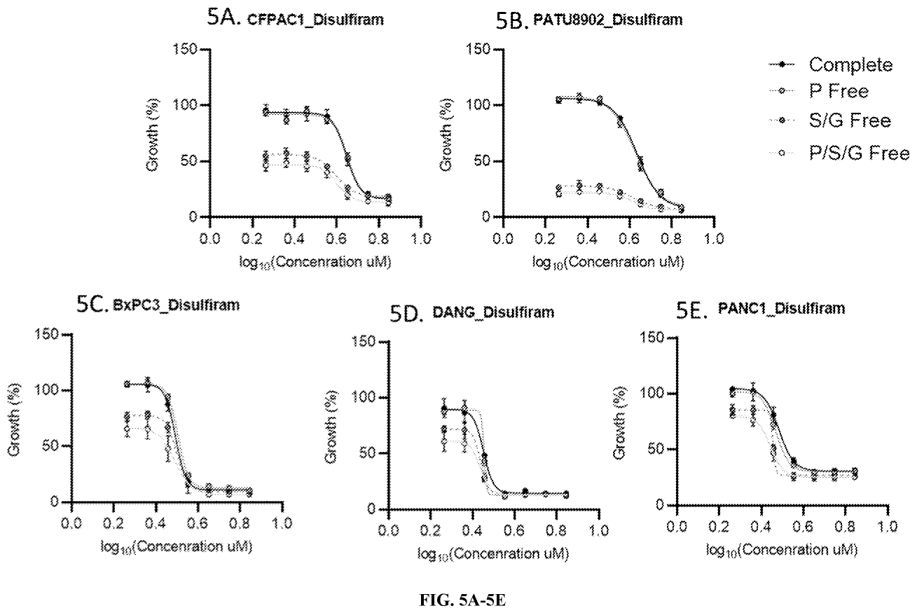

A study was performed to evaluate the efficacy of a disclosed composition in the treatment of cancer when combined with disulfiram (Antabuse™). Pancreatic cancer cell lines BxPC3, CFPAC1, PANC1, DANG and PATU8902 were treated with disulfiram, which is a drug used to treat alcohol addiction but no established use in cancer treatment. Cells were seeded in 96-well plates and in complete media+10% FBS. 24 hours later, cells were washed with PBS followed by addition of disulfiram with either complete or P, S/G or P/S/G free media+10% dialyzed FBS. Cells were grown for 3 more days, and cell growth was measured using Crystal Violet staining. Error bars denote the standard deviation. FIG. 5A-5E illustrate that S/G restriction improved cell kill in response to disulfiram, and that P/S/G restriction further sensitized cell kill by disulfiram, indicated by the response curves being pushed down and to the left with respect to complete media.

### Example 9: Study to Evaluate a Composition Disclosed Herein for Treatment of Cancer in Combination with Targeted Anti-Cancer Therapy

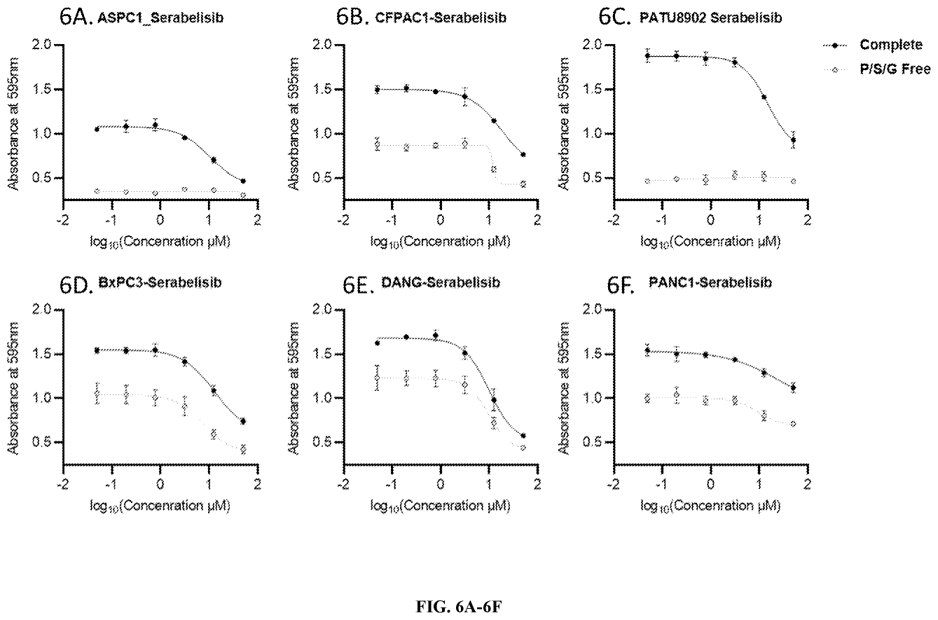

A study was performed to evaluate the efficacy of disclosed compositions in the treatment of cancer when combined with a targeted anti-cancer therapy. Pancreatic cancer cell lines such as BxPC3, CFPAC1, PANC1, DANG and PATU8902 were treated with an alpha specific PI3K inhibitor, serabelisib. Cells were seeded in 96-well plates and in complete media+10% FBS. 24 hours later, cells were washed with PBS followed by addition of serabelisib with either complete or P/S/G free media+10% dialysed FBS (FIG. 6A-6F). Cells were grown for 3 more days and cell growth was measured using Crystal Violet staining. Error bars denote standard deviation. FIG. 6A-6F illustrate that P/S/G starvation resulted in improved cell kill at lower drug concentrations. P/S/G starvation pushes the response curve down and to the left meaning lower concentrations of drug achieve the same cell kill.

### Example 10: Study to Evaluate the Relationship Between Serine and Glycine Synthesis Enzymes and Proline Synthesis Enzymes in Pancreatic Cancer Cells

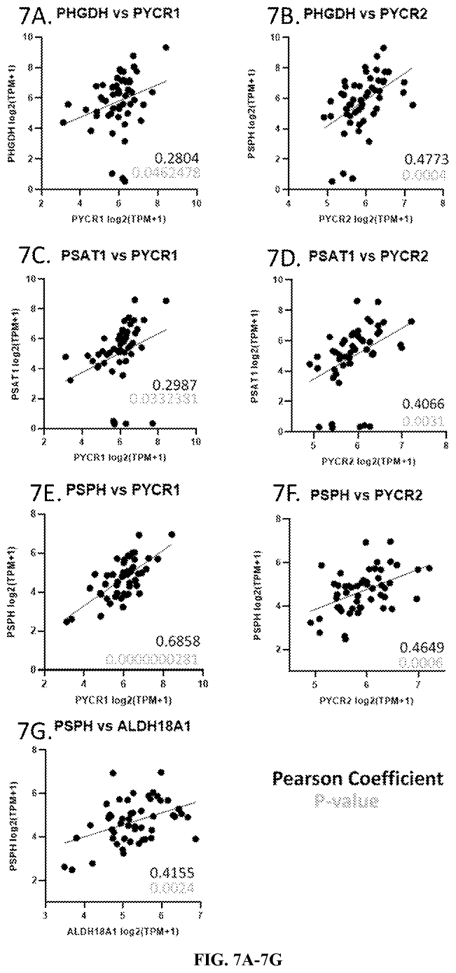

The expression of serine and glycine synthesis enzymes was examined for correlation with proline synthesis enzymes in pancreatic cancer cell lines as illustrated in FIG. 7A-7G. Pearson Correlation analysis between each pair of enzymes showed significant correlation of serine and glycine synthesis with proline synthesis. The mRNA expression of serine and proline biosynthetic enzymes in pancreatic cancer cell lines was obtained from the DepMap database. Pearson Correlation analysis was performed between each pair of enzymes. Significantly correlated enzymes are depicted in FIG. 7A-7G. FIG. 7A illustrates the correlation between PHGDH vs PYCR1; FIG. 7B illustrates the correlation between PHGDH vs PYCR2; FIG. 7C illustrates the correlation between PSAT1 vs PYCR1; FIG. 7D illustrates the correlation between PSAT1 and PYCR2; FIG. 7E illustrates the correlation between PSPH vs PYCR1; FIG. 7F illustrates the correlation between PSPH vs PYCR2; and FIG. 7G illustrates the correlation between PSPH vs ALDH18A1. PYCR3 was not correlated with any SSP enzymes; and ALDH18A1 was only correlated with PSPH. The data showed a correlation between expression of enzymes of de novo serine synthesis and expression of enzymes for de novo proline synthesis, and demonstrate that common mechanisms (e.g., ATF4 and GCN2 signalling) regulate both serine and proline synthesis.

### Example 11: Clinical Trial to Evaluate a Composition Disclosed Herein for Treatment of Pancreatic Cancer in a Subject when Combined with a First Line Drug Therapy

A phase two single arm study is performed to assess the effect of a composition disclosed herein on treating pancreatic cancer in a subject when combined with an Food and Drug Administration (FDA)-approved cancer treatment drug. Sixty subjects with metastatic pancreatic cancer are recruited at ten sites to participate in the study. Subjects with cancer cells that express low levels of enzymes that produce serine/glycine are specifically selected for enrollment due to the increased susceptibility of these cells to proline/serine/glycine restriction. Subjects entered in the trial are administered a dietary product containing a composition disclosed herein, where the dietary product includes all essential/nonessential amino acids and nutrients except for proline, serine, and glycine and is referred to as a Non-Essential Amino Acid Restriction (NEAAR) food.

The enzymes responsible for biosynthesis of serine, are phosphoglycerate dehydrogenase (PHGDH), phosphoserine aminotransferase 1 (PSAT1), and phosphoserine phosphatase (PSPH). Additional enzymes can further convert serine to glycine, i.e., SHMT1 and SHMT2, which interconvert serine and glycine. PSPH expression is relatively constant in tumor cells, whereas PHGDH and PSAT1 expression vary substantially from tumor to tumor. A CAP-CLIA (College of American Pathologists, Clinical Laboratory Improvement

Amendments 1998) validated immunohistochemical assay to detect tumor tissue expression of PHGDH and PSAT1 is employed to identify qualifying subjects, whom are ranked by tumor expression of PHGDH and PSAT1.

Subjects are eligible to be included only if all the following criteria apply:
1. Subjects presenting with histologically or cytologically confirmed metastatic pancreatic adenocarcinoma diagnosed within 6 weeks prior to screening.
2. Subjects are eligible for and being scheduled to begin treatment with the gemcitabine and nab-paclitaxel (gem+nabP) regimen.
3. Subjects who are 18 years of age or older.
4. Subjects are capable of giving signed informed consent.
5. Females of child-bearing potential have a negative serum pregnancy test (β-hCG) result at screening.
6. Subjects with measurable disease within the last 3 weeks prior to screening as determined by RECIST 1.1.
7. Eastern Cooperative Oncology Group (ECOG) Performance Status of ≤1.
8. A subject that has adequate organ function during screening evaluations is defined as all the following:
   a. Absolute neutrophil count (ANC)≥1.5×10⁹/L (1500/μL).
   b. Platelet count≥100×10⁹/L.
   c. Hemoglobin ≥9 g/dL.
   d. Activated partial thromboplastin time/prothrombin time/international normalized ratio (aPTT/PT/INR)≤1.5× upper limit of normal (ULN) unless the subject is on anticoagulants in which case therapeutically acceptable values (as determined by the investigator) meet eligibility requirements.
   e. Aspartate aminotransferase (AST) or alanine aminotransferase (ALT)≤2.5× ULN. In the case of known (i.e., radiological or biopsy documented) liver metastasis, serum transaminase levels must be≤5×ULN.
   f. Total serum bilirubin≤1.5×ULN (except for subjects with known Gilbert's Syndrome for which≤3×ULN is permitted).
   g. Serum creatinine <2.0×ULN and creatinine clearance ≥50 mL/min/1.73 m².
   h. Serum albumin ≥3.5 mg/dL.
9. Subjects must express elevated levels of carbohydrate antigen 19-9 (CA 19-9), carcinoembryonic antigen (CEA), and/or carbohydrate antigen 125 (CA125) during screening.
10. Subjects must have normal Vitamin D levels or be willing to start Vitamin D supplementation during the NEAAR medical food period.
11. Subjects must have available pancreatic ductal adenocarcinoma (PDAC) tissue samples and provide consent for them to be obtained and analyzed by the study sponsor to assist in determining eligibility. A minimum of five (ten preferred) FFPE archival or fresh tumor tissue slides are required.
12. Confirmation of low expression of enzymes that manufacture serine/glycine as assessed by IHC on the FFPE archival or fresh tumor tissue slides provided by the site.
13. Subjects must be willing to stop taking any supplements, herbal medicines, or alternative remedies or other prescribed or over the counter supplements for at least 1 week prior to Cycle 1 Day 1 of gem+nabP and through the NEAAR medical food period.

Subjects are excluded from the study if any of the following criteria apply:
1. Any prior neoadjuvant or adjuvant therapy for pancreatic cancer within 6 months of screening.
2. Comorbidity risk, that in the discretion of the investigator would make the subject a poor candidate for the NEAAR medical food.
3. Diagnosis of another malignancy within the past 2 years (excluding a history of carcinoma in situ of the cervix, superficial non-melanoma skin cancer, or superficial bladder cancer that has been adequately treated, or stage 1 prostate cancer that does not require treatment or requires only treatment with luteinizing hormone-releasing hormone agonists or antagonists if initiated at least 30 days prior to beginning the NEAAR medical food).
4. A body mass index (BMI)<20 kg/m² or >40 kg/m² or, serious or refractive cachexia or anorexia that, in the investigator's opinion, realistically prohibits subjects from having energy or appetite sufficient to reliably engage in a strict medical food regimen for an extended time.
5. Insulin-dependent diabetes.
6. Subjects who must take medications that impact proline, serine, glycine levels (refer to TABLE 2 for a list of commonly known medications that impact proline, serine, and glycine levels).
7. Inability or unwillingness to comply with study and/or follow-up procedures, or medical food modifications described in the protocol.
8. Presence of any significant comorbidity including clinically significant cardiac disease (e.g., congestive heart failure, symptomatic coronary artery disease and cardiac arrhythmias not well controlled with medication), myocardial infarction or unstable angina within the 12 months prior to screening, or any major organ failure.
9. Known hypersensitivity, intolerance, or religious restrictions regarding pork or pork-derived products or to any of the components of the medical food modification, gemcitabine or nab-paclitaxel, pancreatic enzyme replacement therapy (PERT) products (e.g., Pancreaze), or formulary excipients in these products.
10. Untreated clinically significant hyperlipidemia per investigator.
11. Subjects with a condition (including gallbladder disease and/or fatty acid oxidation disorders) where high-fat or fatty food is contraindicated.
12. Any non-cancerous co-existing condition that could elevate CA 19-9, CEA, or CA125.
13. Presence of central nervous system or brain metastases that are not controlled under treatment as assessed by the investigator.
14. Presence of any condition (e.g., persistent diarrhea) that renders the subject unable to satisfactorily chew, swallow, digest, or tolerate the majority of foods and liquids of the NEAAR medical food.
15. Taking or needs to take any protein or amino acid containing nutritional supplements (e.g., Ensure®).
16. Women who are, plan to be, or may potentially be pregnant or lactating.
17. Lack of physical integrity of the upper or lower gastrointestinal (GI) tract or malabsorption syndrome.
18. Known, existing uncontrolled coagulopathy.
19. Major surgery or significant traumatic injury within 14 days of planned start of NEAAR medical food or the anticipation of the need for a major surgical procedure during the study.
20. Active, clinically significant, uncontrolled bacterial, viral, or fungal infection(s).
21. Known current infection with human immunodeficiency virus (HIV), hepatitis B, or hepatitis C.
22. History of confirmed food allergy.
23. Currently enrolled in any other investigational trial or treatment with investigational therapy (ies).
24. Diagnosed with an eating disorder, irritable bowel syndrome (IBS), Crohn's disease, ulcerative colitis, gluten-sensitive enteropathy, or other malabsorption syndrome.
25. Not currently taking or not willing or able to take an FDA-approved Pancreatic Enzyme Replacement Therapy (PERT) during the NEAAR medical food period.

TABLE 2
Medication or Supplement Name

Collagen
Cartilage
Hyaluronic acid
Probiotics
Turmeric with bioperine (or black pepper extract)
Supplements that contain gelatin as an ingredient
Liquid medications (over-the-counter or prescribed) will be avoided, given
that they are often formulated with flavoring and/or sweetening agents
Fiber supplements (e.g., Citrucel) should be avoided. Miralax (i.e.,
polyethylene glycol) is recommended if required.
Lithium (may cause lithium blood levels to rise as result of water loss
during transition)
Antipsychotic medications such as risperidone (Risperdal-Janssen),
aripiprazole (Abilfy-Otsuka), and quetiapine fumarate (Seroquel -
Astrazeneca)
Epilepsy drugs, especially divalproex sodium, zonisamide, and topiramate.

Study Procedure: Each subject's plasma proline, serine, and glycine concentrations and metabolic health are tracked over time. Subjects are administered standardized medical food containing a composition disclosed herein, where the medical food consists of all essential/nonessential amino acids and nutrients except for proline, serine, and glycine. The standardized medical food is referred to as Non-Essential Amino Acid Restriction (NEAAR) food. Subjects are treated once a week with FDA-approved first line combination therapy of gemcitabine and nab-paclitaxel (gem+nabP). Subjects are required to consume the NEAAR medical food for 5 consecutive days out of 7 days each week. The 5 consecutive days must include 1 day before chemotherapy administration (gem+nabP) and the day of chemotherapy administration. For example, if gem+nabP is administered on a Tuesday, NEAAR medical food is consumed Monday, Tuesday, Wednesday, Thursday, Friday and subjects eat their habitual food on Saturday and Sunday.

On days of habitual diet there are no specific prescribed limits or strict guidelines about what a subject can and cannot eat; subjects are encouraged to eat a ‘balanced diet’ containing protein, carbohydrates, and fat. On days of habitual diet, subjects complete a daily Food Quality and Quantity Questionnaire. Allowing subjects to consume habitual food on 2 out of 7 days encourages full compliance during the 5 out of 7 days with the NEAAR medical food, because subjects can look forward to having a regular ‘break’ from the NEAAR medical food.

One mechanism of treatment by the NEAAR medical food is that it can make a tumor more sensitive to other therapies. Consequently the NEAAR medical food is given prior, during, and after chemotherapy. Subjects take gem+nabP one day per week because there are fewer potential benefits of taking the NEAAR medical food more than 4 days after the chemotherapy dose. The maximum potential management of a subject's disease is mechanistically predicted to be greatest on days before, during, and just after chemotherapy is taken. Metabolic adaptation (of systemic and/or tumor metabolism) to the NEAAR medical food is minimized by the intermittent schedule, which produces a greater benefit on disease management than giving the NEAAR medical food in a constant fashion.

Assessment of Subjects and Efficacy: Efficacy is monitored through CT scans with contrast and pancreatic cancer specific biomarkers over the study time points. A targeted physical examination includes, at a minimum, assessments of the gross neuro, heart, lungs, abdomen, and extremities. Height (initial screening only) and weight are measured and recorded at each visit. During study visits the subject is asked if they have been compliant with the NEAAR medical food protocol and the subject's answers are recorded. Compliance is further assessed by the subject Food Quality and Quantity questionnaire and via the subject's amino acid level assessments. Investigators document and review laboratory results to assess any abnormal results, which are reported as either clinically significant (CS) or not CS (NCS). Laboratory abnormalities are not automatically regarded as an adverse event (AE) unless accompanied by signs or symptoms related to the abnormal lab.

Subjects answer questionnaires both on and off site. The body composition questionnaire is completed at baseline and weekly by the subject and consists of entering the subject's weight and body fat percentage after using a provided scale as instructed. Subjects answer a daily questionnaire on food quality, quantity, and taste of each meal consumed on both NEAAR food and habitual food days. Subjects answer a monthly questionnaire on their general quality of life, including general well-being, physical activity, social activities, emotional state, and symptoms of fatigue and appetite. The Eastern Cooperative Oncology Group (ECOG) Performance Status scale is used to assess disease progression and how it affects activities of daily living at each site visit. The scale is a Grade from 0 meaning fully active to 5 for deceased. Pancreatic cancer biomarkers, amino acid profiles, and archived or fresh tumor samples are collected at each site visit.

Adverse Events: Subjects are carefully monitored for AEs from the time of consent through Study Discontinuation. The investigator is responsible for identifying, documenting, and recording events that meet the definition of an AE or SAE and are responsible for following all AEs, including AEs that are serious, considered related to the NEAAR medical food or study procedures, or that caused the subject to discontinue. AEs are assessed in terms of seriousness, severity, expectedness, relationship to the NEAAR medical food (causality), and outcome. Severity is graded according to the scale outlined in TABLE 3.

TABLE 3
Adverse Event (AE) Severity Assessment

| Grade | Description |
|---|---|
| Grade 1 | Mild; symptomatic or mild symptom; clinical or diagnostic |
| | observations only; intervention not indicated. |
| Grade 2 | Moderate; minimal, local or noninvasive intervention indicated; |
| | limiting age-appropriate instrumental activities of daily living |
| | (e.g., preparing meals, shopping for groceries or clothes, using |
| | the telephone, managing money, etc.). |
| Grade 3 | Severe or medically significant but not immediately life- |
| | threatening; hospitalization or prolongation of hospitalization |
| | indicated; disabling; limiting self-care activities of daily living |
| | (e.g., bathing, dressing and |
| | undressing, feeding self, using the toilet, taking medications, |
| | etc.). Of note, the term “severe” does not necessarily mean |
| | “serious” in terms of AE classification. |
| Grade 4 | Life-threatening consequences; urgent intervention indicated. |
| Grade 5 | Death related to AE. |

An adverse event is any untoward medical occurrence associated with the use of an intervention in humans, whether or not considered intervention related. AEs are further defined as those untoward signs or symptoms that are not otherwise related to the subject's underlying medical conditions, comorbid condition present at the time of enrollment, or commonly known side effects from any concomitant therapeutics.

An adverse event is classified as serious (SAE) if the AE results in any of the following:
Death.
Life threatening.
Subject was at substantial risk of dying at the time of the AE or use or continued use of the NEAAR medical food might have resulted in death.
If the AE resulted in admission to hospital or prolonged a hospitalization. Emergency room visits that do not result in admission to hospital are not considered serious unless the event met one of the other criteria for seriousness. Planned hospitalization for a pre-existing condition without serious deterioration in health is not considered an SAE.
Disability or Permanent Damage.
If the AE resulted in a substantial disruption of a person's ability to conduct normal life functions, i.e., the AE resulted in significant, persistent or permanent change, impairment, damage or disruption in the subject's body function/structure, physical activities, and/or QOL.
Congenital Anomaly/Birth Defect-if it is suspected that exposure to the NEAAR medical food prior to conception or during pregnancy may have resulted in an adverse outcome in the child.

Other events (Important Medical Events) are considered serious when the event does not fit the other outcomes, but the event may jeopardize the subject and may require medical or surgical intervention (treatment) to prevent one of the other outcomes. Examples include allergic bronchospasm (a serious problem with breathing) requiring treatment in an emergency room, serious blood dyscrasias (blood disorders) or seizures/convulsions that do not result in hospitalization. The development of drug dependence or drug abuse is also an example of important medical events. Disease progression is not considered an AE or SAE unless the Investigator feels that the progression was clearly unusual for pancreatic cancer in quantitative or qualitative aspects.

Study Schedule: Pre-trial Visit-3 weeks prior to expected Cycle 1/Day 1 of gem+nabP administration (Initial Screening): Subjects are screened for eligibility and considered enrolled when informed consent is signed. The following study procedures should be performed within 3 weeks of the expected first administration of gem+nabP unless otherwise indicated:
Informed Consent
Confirmation of eligibility (review and confirmation of inclusion/exclusion criteria)
Confirmation, request, and shipping of tumor tissue from biopsy. A minimum of five (10 preferred) formalin fixed paraffin-embedded (FFPE) archival tumor tissue or fresh tumor tissue slides are required; the study team may request more material, e.g., 10 FFPE slides, or a FFPE block if available. This tissue is sent as soon as possible to a central lab for quantification of metabolic enzyme expression via IHC assay to confirm low expression (eligibility for study) or normal expression (ineligible for study).
Recording of demographics, medical history, and record of medications.
Targeted physical exam
Vital signs, height, and weight
CT Scan with contrast (protocol per standard of care with 5 mm cuts of the chest, abdomen, and pelvis). CT scans are repeated during the study per standard of care every 4-8 weeks.
ECOG assessment
Laboratory collection for: CBC w/diff, CMP, Urinalysis, Serum hCG for women of childbearing potential (any premenopausal female capable of becoming pregnant) must have a negative pregnancy test, Lipid Panel, CA19-9, CEA, and/or CA125, Magnesium, Vitamin D
Blood draw for Amino Acids (Proline, Glycine, Serine) preferably 2-3 hours after the subject's last habitual meal.

Eligible subjects proceed as follows: subject returns to the site for a second blood draw for Amino Acids (Proline, Glycine, Serine), 2-3 hours after the subject's habitual breakfast or lunch. Pancreatic Enzyme Replacement Therapy (PERT): The subject is assessed as to whether subject is already taking PERT and if not, PERT is initiated at time of the NEAAR medical food run-in period. A smart scale is provided to the subject and the subject receives a call from a registered dietician. During this call, the dietician: Reviews the subject's dietary preferences, instructs the subject on how to operate their smart scale, instructs the subject on use of a HIPAA-compliant App (the App), and gives specific medical food instructions. The App allows the subject to communicate with the registered dietitian regarding any nutrition-related concerns that may interfere with their ability to comply with the NEAAR medical food. The subject is provided with study supplies prior to their first dose of gem+nabP. Subjects ingest the NEAAR medical food for a minimum of 3 days prior to the subject's first dose of gem+nabP.

First Visit-Cycle 1/Day 1 of gem+nabP administration: The subject receive his or her first dose of gem+nabP per standard of care. The following procedures occur during this visit:
Targeted physical exam
Vital signs and weight
ECOG assessment
Laboratory collection for: CBC w/diff, CMP, Urinalysis, Urine hCG for women of childbearing potential, Lipid Panel, Magnesium
Blood draw for Amino Acids (Proline, Glycine, Serine): the timing of this blood draw is dependent on when the subject's last NEAAR medical food was ingested and preferably should occur 2-3 hours after a NEAAR medical food consumption.
Assessment of NEAAR medical food tolerability and compliance
Concomitant medications including PERT
Adverse Event Assessment

Second Visit—Cycle 1/Day 8—2^{nd} gem+nabP administration (expected)/1 week after Study Visit 1: The subject receives his or her 2^{nd} dose of gem+nabP per standard of care. If the chemotherapy is held, the study visit still proceeds. The following procedures occur during this visit:
Targeted physical exam
Vital signs and weight
ECOG assessment
Laboratory collection for (before chemotherapy if applicable): CBC w/diff, CMP, Urinalysis, Urine hCG for women of childbearing potential
Blood draw for Amino Acids (Proline, Glycine, Serine): the timing of this blood draw is dependent on when the subject's last NEAAR medical food was and preferably should occur 2-3 hours after a NEAAR medical food consumption.
Assessment of NEAAR medical food tolerability and compliance.
Concomitant medications including PERT
Adverse Event Assessment

Third Visit—Cycle 1/Day 15—3^{rd} gem+nabP administration (expected)/1 week after Study Visit 2: The subject receives his or her 3^{rd} dose of gem+nabP per standard of care. If the chemotherapy is held, the study visit still proceeds. The following procedures occur during this visit:
Targeted physical exam
Vital signs and weight
ECOG assessment
Laboratory collection for (before chemotherapy if applicable): CBC w/diff, CMP, Urinalysis, Lipid Panel, Magnesium, Urine hCG for women of childbearing potential, CA19-9, CEA, and/or CA125 (done monthly, may occur at a different visit per site SOC but must be done at least monthly from baseline/screening visit)
Blood draw for Amino Acids (Proline, Glycine, Serine): the timing of this blood draw is dependent on when the subject's last NEAAR medical food was and preferably should occur 2-3 hours after a NEAAR medical food consumption.
Assessment of NEAAR medical food tolerability and compliance.
Concomitant Medications including PERT
Adverse Event Assessment

Alternate Subsequent Visits-Day 1 of each subsequent cycle: On Day 1 of each cycle of the subject's chemotherapy regimen the following procedures occur:
Targeted physical exam
Vital signs and weight
ECOG assessment
Laboratory collection (before chemotherapy if applicable) for: CBC w/diff, CMP, Urinalysis, Urine hCG for women of childbearing potential.
Blood draw for Amino Acids (Proline, Glycine, Serine): the timing of this blood draw is dependent on when the subject's last NEAAR medical food was and preferably occurs 2-3 hours after a NEAAR medical food consumption.
Assessment of NEAAR medical food tolerability and compliance.
Concomitant Medications including PERT
Adverse Event Assessment

Alternate Subsequent Visits—Day 15 of each subsequent cycle: On Day 15 of each cycle of the subject's chemotherapy regimen the following procedures occur:
Targeted physical exam
Vital signs and weight
ECOG assessment
CT Scan with contrast (protocol per standard of care with 5 mm cuts of the chest, abdomen, and pelvis). CT scans are repeated during the study per standard of care every 4-8 weeks and therefore may occur at a different visit.
Laboratory collection (before chemotherapy if applicable) for: CBC w/diff, CMP, Urinalysis, Lipid Panel, Magnesium, Urine hCG for women of childbearing potential, CA19-9, CEA, and/or CA125
Blood draw for Amino Acids (Proline, Glycine, Serine): the timing of this blood draw is dependent on when the subject's last NEAAR medical food was and preferably should occur 2-3 hours after a NEAAR medical food consumption.
Assessment of NEAAR medical food tolerability and compliance.
Concomitant Medications including PERT
Adverse Event Assessment

Final Visit 10 days±3 days after NEAAR medical food ends: The Final Study Visit takes place at a study site after the subject has completed or discontinued the NEAAR medical food and the following final safety assessments are performed:
Targeted physical exam
Vital signs and weight
ECOG assessment
CT scan if not performed within the last 4-8 weeks
Laboratory collection (before chemotherapy if applicable) for: CBC w/diff, CMP,

Urinalysis, Lipid Panel, Magnesium, Urine hCG for women of childbearing potential, CA19-9, CEA, and/or CA125
Assessment of NEAAR medical food tolerability and compliance if applicable.
Concomitant Medications including PERT
Adverse Event Assessment
Study Discontinuation

Primary outcome: The primary outcome is a measurement of the percent of Grade 3 or 4 adverse event rate observed in the NEAAR medical food and gem+nabP regimen for comparison with gem+nabP alone, which was 38% as reported in the MPACT trial. These data indicate a lower incidence rate of adverse events than 38%, demonstrating the efficacy of NEAAR food in improving cancer treatment.

### Secondary Outcomes:

Overall Response Rates (defined as complete response [CR] and partial response [PR] using the Response Evaluation Criteria in Solid Tumors guidelines (RECIST) v1.1).
Absolute and relative change from baseline of biomarkers of interest at each assessment visit using the modified Intention-To-Treat (mITT) population.
A biomarker analysis on the percent change from baseline to last available observation using the mITT population.
Progression Free Survival (duration from radiographic documentation of disease to radiographic documentation of progression or death from any cause) using RECIST 1.1 and as determined by Investigator Review using the mITT population.
Overall survival using Kaplan-Meier; (defined as time in months from the date of first study treatment to the date of death) using the mITT population.
Measurement of Changes in Eastern Cooperative Oncology Group (ECOG) performance score and weight at each available time point from baseline to last available time point using the mITT population.
Measurements of descriptive statistics at baseline and all available timepoints and change from baseline at each timepoint and to last available time point using absolute and relative values using the mITT population.
Correlation and regression analysis with amino acid concentrations using the mITT population.
Comparison of palatability of the NEAAR medical food versus habitual food.
Measurement of compliance with diet through daily food questionnaire.

### Example 12: Double-Blind Randomized Controlled Trial to Evaluate a Composition Disclosed Herein for Treatment of Pancreatic Cancer in a Subject when Combined with First Line Drug Therapies

A multi-center, placebo-controlled, double-blind, randomized clinical trial is conducted to assess the sensitization of pancreatic cancer cells to chemotherapy by a composition disclosed herein. The patient population, intervention, inclusion/exclusion criteria, assessments and outcomes are identical to those described in EXAMPLE 11, with the exception that patients are randomly assigned to two groups: the NEAAR medical food group and the control group. The control group is administered standardized medical food that is identical to the NEAAR food, except that it provides all essential and nonessential amino acids. Results are directly compared between the NEAAR group and the control group to determine the therapeutic effects of the NEAAR food in combination with gem+nabP for the treatment of cancer.

### Example 13: Single Arm Medical Food Study to Evaluate a Standardized Nonessential Amino Acid Restrictive Medical Food for the Dietary Management of Metastatic Colorectal Cancer

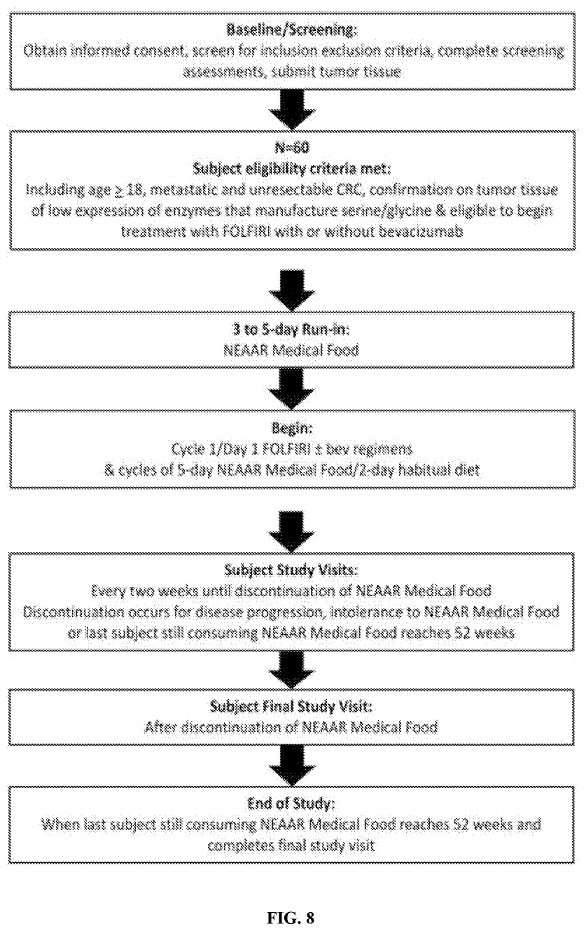

A single arm study is performed to evaluate the tolerability of a specially designed medical food restricted in proline, serine, and glycine for the dietary management of subjects with metastatic colorectal cancer. Subjects receive, per standard-of-care (SOC), a regimen (FOLFIRI±BEV) comprised of FDA-approved second line drug therapies that are routinely prescribed for metastatic colorectal cancer (mCRC). Subjects whose tumors express low levels of the enzymes that manufacture serine or glycine are eligible. FIG. 8 outlines the stages of the metastatic colorectal cancer clinical trial from baseline/screening to end of study.

Subjects with metastatic CRC who have failed frontline therapy are recruited at up to ten sites in the United States. Subjects are ≥18 years old with histologically confirmed metastatic and unresectable CRC that have failed treatment for fluoropyrimidine and oxaliplatin±BEV and have confirmation of tumor tissue of low expression of enzymes that manufacture serine/glycine and are eligible to begin treatment with FOLFIRI±BEV. Subjects receive the NEAAR medical food, and the primary objective is to demonstrate tolerability of the NEAAR medical food when administered concomitantly with chemotherapy in the management of metastatic colorectal cancer.

Subjects undergo a screening visit for eligibility and have baseline assessments including a CT scan of their chest, abdomen, and pelvis, baseline plasma amino acids, and baseline safety labs. The site requests a sample of archived or fresh tumor tissue from a primary or metastatic site that has been biopsied within the last 6 months for eligibility assessment based on the expression of enzymes that are responsible for manufacturing serine/glycine. Once deemed eligible, subjects complete a three to five-day run-in period with the NEAAR medical food immediately prior to starting FOLFIRI±BEV SOC. The subjects continue a seven-day schedule of the NEAAR medical food for five days followed by two days of the subject's habitual food until documented disease progression, occurrence of intolerable adverse effects, desire to stop the NEAAR medical food, or withdrawal of consent. Alternatively, subjects complete a one to three-day run-in period with the NEAAR medical food immediately prior to starting FOLFIRI±BEV SOC. Then, the subjects continue an intermittent schedule of at least two days of the NEAAR medical food with the remaining days on habitual food. Subjects who tolerate the NEAAR medical food and whose disease has not progressed continue to receive the NEAAR medical food until End of Study (EOS). Subjects have visits with the study team designed to coincide with their scheduled SOC FOLFIRI±BEV administration when possible. Study visits occur weekly for the first three weeks following initiation of the NEAAR medical food and then every two weeks until discontinuation of the NEAAR medical food. Subjects have a final study visit for safety assessments approximately 10 days after discontinuation of NEAAR medical food. If a subject experiences an SAE near the time of NEAAR medical food discontinuation, the subject is followed for at least 30 days. Subjects continue to adhere to the 5 day on and 2 day off medical food regimen during the weeks that they do not receive chemotherapy. Treatment emergent adverse events (TEAEs) are assessed. During the study, the subjects complete dietary and Quality of Life (QOL) questionnaires and record weekly weights and body fat percentage measurements via a Health Insurance Portability and Accountability Act (HIPAA) compliant App.

The first 10 subjects with complete cycle 1 data are reviewed for limiting toxicities by a Safety Monitoring Committee (SMC) as part of an interim safety assessment. Individual subject and study stopping rules apply as appropriate. The SMC continues to assess subject safety data throughout the study. Metabolomic impact of the NEAAR medical food is assessed via an exploratory metabolomic analysis that compares the plasma sample taken during screening with a plasma sample while on study. Management of disease with the NEAAR medical food while on chemotherapy is assessed via an exploratory analysis that compares the plasma sample taken for ctDNA during screening with a plasma sample taken at study visits 3 and 7.

The trial collects data from routine/standard treatment that subjects receive and data from administration of the investigational product. The primary objective for the study is to demonstrate tolerability of the medical food. Secondary objectives include assessing disease management with the medical food and looking at changes in plasma amino acid concentrations. Exploratory endpoints include comparing gene mutation and gene expression as predictors of management of disease with the NEAAR medical food; compare palatability of the NEAAR medical food versus habitual food; and measure changes in metabolomic markers or ctDNA as a result of the NEAAR medical food.

The primary endpoint is the rate of the most common Grade 3 and 4 adverse event related to the NEAAR medical food (when added to standard of care FOLFIRI±BEV) regimen. This rate is compared with the rate of the most common Grade 3 and 4 adverse event of the FOLFIRI±BEV regimen.

The secondary endpoints include: 1) Demonstrate impact of the NEAAR medical food on disease management markers—1a) Overall Response Rates (defined as complete response [CR] and partial response [PR] using the RECIST 1.1) based on the modified intent to treat (mITT) populations; 1b) Absolute and relative change from baseline of biomarkers of interest at each assessment visit using the mITT population; 1c) A biomarker analysis is performed on the percent change from baseline to last available observation using the mITT population; 1 d) Progression-free Survival (PFS) (duration from radiographic documentation of disease to radiographic documentation of progression or death from any cause) at 6, 9, and 12 months using RECIST 1.1 using the mITT population. 2) Assess clinical benefit measures (weight, Eastern Cooperative oncology Group [ECOG] performance status) for the time the subject is consuming NEAAR medical food-Changes in ECOG performance status and weight at each available time point from baseline to last available time point using the mITT population. 3) Study all plasma amino acid concentrations for the time the subject is consuming NEAAR medical food-3a) Measuring descriptive statistics at baseline and all available timepoints and change from baseline at each timepoint and to last available time point using absolute and relative values using the mITT population; 3b) Correlation and regression analysis with amino acid concentrations using the mITT population.

Exploratory endpoints include: 1) Comparison of gene mutation and gene expression as predictors of disease management with NEAAR medical food. Available tumor tissue is analyzed by immunohistochemistry for metabolic enzyme expression; any pre-existing genomic data (DNA sequencing, RNA expression analysis) is reported; and any data in subjects where genomic analysis has not been previously performed (DNA sequencing, RNA expression analysis) is reported. 2) Compare palatability of the NEAAR medical food versus habitual food. Compliance with the diet is monitored through daily food questionnaires; and 3) metabolomic biomarkers and ctDNA and relationships are monitored with the management of disease with NEAAR medical food.

Inclusion criteria are substantially similar to those of EXAMPLE 11, except that the subject must have failed treatment for fluoropyrimidine and oxaliplatin±BEV. All subjects must have received a minimum of 6 weeks of a first line regimen that included oxaliplatin and a fluoropyrimidine±BEV. Treatment failure is defined as radiologic progression during or <6 months after the last dose of first-line therapy. Subjects who show tumor progression while on maintenance therapy with a fluoropyrimidine±BEV after prior fluoropyrimidine-oxaliplatin±BEV induction therapy are eligible. Re-challenge with oxaliplatin is permitted and is considered part of the first-line regimen for metastatic disease, with both initial oxaliplatin treatment and subsequent re-challenge being considered as one regimen. Subjects who received oxaliplatin/fluoropyrimidine-based neoadjuvant or adjuvant therapy and have disease recurrence or progression >6 months from their last dose of neoadjuvant or adjuvant treatment (or >6 months from surgery if no adjuvant therapy was administered) are required to receive and progress on fluoropyrimidine/oxaliplatin-based therapy±BEV for metastatic disease. For subjects with rectal cancer, sequential neoadjuvant and adjuvant therapy counts as a single systemic regimen. Subjects who discontinued first-line therapy because of toxicity may be enrolled, as long as progression occurred <6 months after the last dose of first-line therapy. Further, subjects must have available CRC tissue samples from a primary or metastatic site that has been biopsied within the last 6 months and provide consent for them to be obtained and analyzed by the study sponsor to assist in determining eligibility. A minimum of five (ten preferred) FFPE archival or fresh tumor tissue slides are required.

Exclusion criteria are substantially similar to those of EXAMPLE 11, except that the subject cannot have concomitant MSI-H/dMMR (Microsatellite Instability High/Deficient Mismatch Repair); Anti-cancer chemotherapy or biologic therapy administered within 3 weeks prior to the first dose of fluoropyrimidine and irinotecan-based regimens. The exception is a single dose of radiation up to 8 Gray (equal to 800 RAD) with palliative intent for pain control up to 14 days before NEAAR medical food and return to baseline or ≤Grade 1 toxicity associated with the radiation therapy. Additional exclusion criteria for BEV include: a) History of cardiac disease: congestive heart failure (CHF) Class II or higher according to the New York Heart Association (NYHA); active coronary artery disease, myocardial infarction within 6 months prior to study entry; unevaluated new onset angina within 3 months or unstable angina (angina symptoms at rest) or cardiac arrhythmias requiring anti arrhythmic therapy (beta blockers or digoxin are permitted); b) Current uncontrolled hypertension (systolic blood pressure [BP]>150 mmHg or diastolic pressure >90 mmHg despite optimal medical management) or prior history of hypertensive crisis or hypertensive encephalopathy; c) History of arterial thrombotic or embolic events (within 6 months prior to study entry); d) Significant vascular disease (e.g., aortic aneurysm, aortic dissection, symptomatic peripheral vascular disease); e) Evidence of bleeding diathesis or clinically significant coagulopathy; f) Major surgical procedure (including open biopsy, significant traumatic injury, etc.) within 28 days, or anticipation of the need for major surgical procedure during the study, and minor surgical procedure (excluding placement of a vascular access device) within 7 days prior to study enrollment; g) Proteinuria at Screening as demonstrated by urinalysis with proteinuria ≥2+ (patients discovered to have ≥2+ proteinuria on dipstick urinalysis at baseline should undergo a 24-hour urine collection and must demonstrate≤1 g of protein in 24 hours to be eligible); h) History of abdominal fistula, GI perforation, peptic ulcer, or intra-abdominal abscess within 6 months; i) Ongoing serious, non-healing wound, ulcer, or bone fracture; j) Known hypersensitivity to any component of BEV (LIST); k) History of reversible posterior leukoencephalopathy syndrome (RPLS).

NEAAR medical food: The NEAAR medical food is a standardized medical food which consists of all essential/nonessential amino acids and nutrients except for proline, serine, and glycine. The medical food consists of two elements: 1) standardized meals and snacks with minimal protein and 2) a plant derived powder to be reconstituted with water that administers all proteinogenic essential and non-essential amino acids except for proline, serine, and glycine. Subjects also take a daily multivitamin as provided by the study sponsor.

Administration of NEAAR medical foods: Subjects ingest the NEAAR medical food for a minimum of 3 days prior to the subject's first dose of FOLFIRI±BEV regimen. Subjects are required to consume the NEAAR medical food for 5 consecutive days out of 7 days each week. The 5 consecutive days include at least 24 hours before SOC chemotherapy administration and the day of chemotherapy administration. For example, if FOLFIRI±BEV is administered on a Tuesday, NEAAR medical food are consumed Monday (at least 24 hours prior to chemotherapy administration), Tuesday, Wednesday, Thursday, and Friday, and subjects eat their habitual food on Saturday and Sunday. On days of habitual diet, there are no specific prescribed limits or strict guidelines about what a subject can and cannot eat; subjects are encouraged to eat a ‘balanced diet’ containing protein, carbohydrates, and fat. On days of NEAAR medical food and habitual diet, subjects complete the daily Food Quality and Quantity Questionnaire. The NEAAR medical food is started at least 24 hours prior to FOLFIRI±BEV to allow 24 hours for amino acid levels to decrease prior to chemotherapy.

Meals, food, and beverage restriction: Subjects are not to be permitted to consume anything other than the provided NEAAR medical food and beverages as described below during the 5 days out of 7 the subject is enrolled. These subjects are allowed to consume their habitual food during the other 2 days of the week. Subjects are instructed to consume meals and the NEEAR medical food at typical times of day (i.e., breakfast, lunch, and dinner), and also take a daily multivitamin. The following beverages are allowed: water, diet soda, coffee, and tea (no sugar or milk; artificial sweeteners are allowed)—maximum daily caffeine intake from any source must be less than 500 mg. Unflavored alcohol alone without a mixer (up to 1.5 oz per day), or mixed with just water, seltzer, or diet soda (e.g., whiskey, gin, tequila, vodka mixed with water or seltzer) is allowed at any time. A single serving of wine (5 oz) or beer (12 oz) is allowed on each of the 2 days when the subject is consuming habitual foods.

Concomitant therapies: Any over the counter and prescription medicines, recreational drugs, vitamins, and/or herbal supplements, oral nutritional supplements (e.g., Ensure®), vaccines, or any other medications that the subject is taking at screening and throughout the study are recorded. Subjects must abstain from taking medications that impact proline, serine, and glycine levels as indicated in TABLE 2 above. TABLE 4 shows the schedule of events for the metastatic colorectal cancer study.

Withdrawal from study: A subject may withdraw at any time or may be withdrawn at any time at the discretion of the investigator for safety, behavioral, or compliance reasons.

TABLE 4

| | | | | | | | | Final |
|---|---|---|---|---|---|---|---|---|
| | | | | | | | | Study |
| | | | | | | | | Visit |
| | | | | Study | Study | Study | | 10 ± 3 |
| | | Run-in: | Study | Visit 2 | Visit 3, | Visit 4, | | days |
| | Screening/ | NEAAR | Visit 1 | 1 week | 5, 7 . . . | 6, 8 . . . | | after |
| | Baseline | Medical | 1^{st} Chemo | after | repeating | repeating | NEAAR | NEAAR |
| | visits² | Food after | dose | cycle 1/ | visits | visits | Medical | Medical |
| | Screening | eligibility | cycle 1/ | day 1 ± | Alternating visits | | Food | Food |
| Study visit¹ | Period | confirmation | day 1 | days | every 2 weeks | | END | END |
| Timing related | Within | Min of days | Cycle 1 | | Subsequent Cycles | | | |
| to expected | weeks of | prior to | Day 1 | Day 7-9 | Day 1 of | Day 1 of | | |
| FOLFIRI ± | cycle 1/ | cycle 1/ | | | cycle 2, | cycle 3, | | |
| BEV regimen | day 1 | day1 | | | 4, 6 . . . | 5, 7 . . . | | |
| dosing¹ | | | | | | | | |
| Informed Consent | X | | | | | | | |
| Inclusion/Exclusion | X | | | | | | | |
| Criteria | | | | | | | | |
| Demographics/ | X | | | | | | | |
| Medical History | | | | | | | | |
| Confirmation, | X | | | | | | | |
| request, and | | | | | | | | |
| shipping of | | | | | | | | |
| tumor tissue | | | | | | | | |
| from biopsy³ | | | | | | | | |
| Targeted physical | X | | X | VS/Weight | X | X | | X |
| exam with VS, | | | | only | | | | |
| Height (BL only), | | | | | | | | |
| Weight⁴ | | | | | | | | |
| Lab: Serum hCG | X | | | | | | | |
| (WOCBP only)⁵ | | | | | | | | |
| Lab: Urine or | | | X | | X | X | | X |
| serum hCG | | | | | | | | |
| (WOCBP | | | | | | | | |
| ONLY)⁵ | | | | | | | | |
| Labs: CMP, CBC | X | | X | CMP and | X | X | | X |
| w/diff, | | | | magnesium | | | | |
| Magnesium, & | | | | only⁷ | | | | |
| Urinalysis | | | | | | | | |
| (Baseline, then | | | | | | | | |
| every 2 weeks | | | | | | | | |
| (SOC))⁶ | | | | | | | | |
| Central Lab: | X2 | | X⁹ | X | X | X | | X |
| Amino Acids | | | | | | | | |
| (Baseline × 2, | | | | | | | | |
| then every | | | | | | | | |
| week × 3 then | | | | | | | | |
| every 2 weeks)⁸ | | | | | | | | |
| Labs: Lipid panel | X | | X | | X | | | X |
| (baseline, visit 1, | | | | | | | | |
| day 1 cycle 2, | | | | | | | | |
| then every 4 | | | | | | | | |
| weeks) | | | | | | | | |
| Central Lab: | X | | | | | X¹⁰ | | |
| metabolomic | | | | | | | | |
| biomarkers | | | | | | | | |
| Central Lab: | X | | | | X¹¹ | | | |
| ctDNA | | | | | | | | |
| Labs: Biomarker | X | Biomarkers ever 4 weeks (SOC) | | | | | | |
| CEA/CA 19-9 | | | | | | | | |
| (Baseline and | | | | | | | | |
| every 4 weeks | | | | | | | | |
| (SOC)) | | | | | | | | |
| CT scan of chest, | X | CT scan every 8 weeks (SOC) | | | | | | |
| abdomen, and | Within 6 | | | | | | | |
| pelvis at baseline | weeks of 1^{st} | | | | | | | |
| and at least every | FOLFIRI ± | | | | | | | |
| 8 weeks per SOC | BEV dose | | | | | | | |
| ECOG | X | | X | X | X | X | | X |
| performance | | | | | | | | |
| status | | | | | | | | |
| Concomitant | X | | X | X | X | X | | X |
| meds | | | | | | | | |
| Adverse events | X | X | X | X | X | X | | X |
| Registered | X | X | | | X | | | |
| dietician subject | | | | | | | | |
| call¹² | | | | | | | | |
| Study | | | | | | | | X |
| discontinuation | | | | | | | | |
| FOLFIRI ± BEV | | | Administration per SOC | | | | | |
| (SOC)¹³ | | | | | | | | |
| NEAAR Medical | | NEAAR Medical Food 5 days on/2 days off | | | | | | |
| Food¹⁴ | | | | | | | | |

Subject completion via the App

| Daily food | X | X | Daily |
|---|---|---|---|
| quality and | | | |
| quantity | | | |
| questionnaire | | | |
| Weekly body | X | X | Weekly |
| composition | | | |
| Monthly quality | X | X | Monthly |
| of life | | | |
| questionnaire | | | |

Abbreviations: BEV = bevacizumab, BL = baseline, CA 19-9 = Serum carbohydrate antigen 19-9, CEA = carcinoembryonic antigen, CBC w/diff = complete blood count with differential, CMP = comprehensive metabolic panel, CT = computerized tomography, ECOG = Eastern Cooperative Oncology Group, FOLFIRI ± BEV = infusional 5-FU and irinotecan ± BEV, hCG = human chorionic gonadotropin, NEAAR = NonEssential Amino Acid Restriction, SOC = standard of care, VS = Vital Signs, WOCBP = women of childbearing potential
¹Subject visits are arranged to coincide with days in which chemotherapy is expected to be administered in 2 week cycles. If chemotherapy schedule changes, site to discuss visit schedule with sponsor. If chemotherapy is held for 7 days or more, the study visit still occurs as an additional study visit. The visit schedule then resumes at the subject's next chemotherapy visit.
²All Screening/Baseline assessments are completed within 3 weeks of expected 1st dose of FOLFIRI ± BEV.
³A minimum of five (10 preferred) FFPE archival tumor tissue or fresh tumor tissue slides is required; the tissue is sent as soon as possible to a central lab for quantification of metabolic enzyme expression via IHC assay to confirm low expression (eligibility for study) or normal expression (ineligible for study).
⁴Height required at Baseline/Screening visit only.
⁵Serum hCG testing at Screening and urine or serum hCG testing at other timepoints are required for all WOCBP. When testing occurs on days where subject is dosed with FOLFIRI ± BEV regimen, results must be available before start of chemotherapy.
⁶On days where subject is dosed with FOLFIRI ± BEV regimen, testing for CMP, CBC w/diff & urinalysis must be completed before start of chemotherapy per SOC.
⁷Only a CMP and magnesium draw is required at Study Visit 2 (no chemotherapy is expected at this visit)
⁸Amino Acid Profile (proline, glycine, & serine) is collected 1.5-3 hours after the subject's breakfast for lunch twice during the Baseline Screening period while on habitual diet, once every week × 3 then every 2 weeks on days ingesting NEAAR Medical Food and once at Final Study Visit (on habitual diet). These samples are processed and shipped to a central laboratory. All other lab testing is process at study sites per SOC.
⁹Blood for amino acid will be collected on Study Visit 1 before chemo.
¹⁰For Study Visit 4 only
¹¹For study Visit 3 and 7
¹²Once eligibility is confirmed, a call between a Registered Dietitian (RD) and the subject will be arranged to review the subject's dietary history, subject diet concerns, provide instructions for use of the App and the medical food. RD will also perform subject “check in” calls (done via phone) bieekly. The call is during the non-study site visit week. The timing and cadence of calls may be adjusted based on subject need and the RD's discretion
¹³Decisions regarding dosing, dose modifications, and administration or discontinuation of FOLFIRI ± BEV regimen is at the discretion of the investigator and the site's standard of care and follows the label for each product in the regimen
¹⁴NEAAR Medical Food is consumed exclusively for at least 3 days (Run-in) prior to the 1st dose of FOLFIRI ± BEV regimen and thereafter in cycles of 5 continuous days of exclusive consumption of NEAAR Medical Food and 2 continuous days of the subject's habitual diet. The schedule will be arranged to ensure the subject is ingesting NEAAR Medical Food on Study Visit days.

### EMBODIMENTS

The following non-limiting embodiments provide illustrative examples of the invention, but do not limit the scope of the invention.

Embodiment 1A. A method comprising: a) isolating a biological sample from a subject, wherein the subject has a cancer; b) after the isolating, quantifying an amount of a biosynthetic metabolic enzyme; and c) after the quantifying, administering a nutrient modulation therapy to the subject.

Embodiment 2A. The method of embodiment 1A, wherein the biological sample is a tumor biopsy sample.

Embodiment 3A. The method of embodiment 1A, wherein the biological sample comprises a circulating tumor cell.

Embodiment 4A. The method of embodiment 1A, wherein the biological sample comprises circulating tumor DNA.

Embodiment 5A. The method of any one of embodiments 1A-4A, wherein the subject is human.

Embodiment 6A. The method of any one of embodiments 1A-5A, wherein the cancer is pancreatic cancer.

Embodiment 7A. The method of any one of embodiments 1A-5A, wherein the cancer is breast cancer.

Embodiment 8A. The method of any one of embodiments 1A-5A, wherein the cancer is colon cancer.

Embodiment 9A. The method of any one of embodiments 1A-8A, wherein the biosynthetic metabolic enzyme is involved in a nutrient biosynthesis.

Embodiment 10A. The method of any one of embodiments 1A-9A, wherein the biosynthetic metabolic enzyme is involved in a nutrient processing.

Embodiment 11A. The method of embodiment 9A or 10A, wherein the nutrient is a fatty acid.

Embodiment 12A. The method of embodiment 9A or 10A, wherein the nutrient is a lipid.

Embodiment 13A. The method of embodiment 9A or 10A, wherein the nutrient is an amino acid.

Embodiment 14A. The method of embodiment 13A, wherein the amino acid is serine.

Embodiment 15A. The method of embodiment 13A, wherein the amino acid is glycine.

Embodiment 16A. The method of any one of embodiments 1A-15A, wherein the biosynthetic metabolic enzyme is PHGDH.

Embodiment 17A. The method of any one of embodiments 1A-15A, wherein the biosynthetic metabolic enzyme is PSAT1.

Embodiment 18A. The method of any one of embodiments 1A-17A, wherein the nutrient modulation therapy comprises administering a therapeutically effective amount of a dietary product.

Embodiment 19A. The method of embodiment 18A, wherein the dietary product is devoid of at least one amino acid.

Embodiment 20A. The method of embodiment 19A, wherein the dietary product is devoid of serine.

Embodiment 21A. The method of embodiment 19A, wherein the dietary product is devoid of glycine.

Embodiment 22A. The method of embodiment 19A, wherein the dietary product is devoid of proline.

Embodiment 23A. The method of embodiment 19A, wherein the dietary product is devoid of tyrosine.

Embodiment 24A. The method of embodiment 18A, wherein the dietary product is devoid of at least two amino acids.

Embodiment 25A. The method of embodiment 24A, wherein the dietary product is devoid of serine and glycine.

Embodiment 26A. The method of embodiment 18A, wherein the dietary product is devoid of at least three amino acids.

Embodiment 27A. The method of embodiment 26A, wherein the dietary product is devoid of serine, glycine, and proline.

Embodiment 28A. The method of embodiment 26A, wherein the dietary product is devoid of serine, glycine, and cysteine.

Embodiment 29A. The method of any one of embodiments 1A-28A, wherein the administering is oral.

Embodiment 30A. The method of any one of embodiments 18A-29A, wherein the therapeutically effective amount is from about 0.5 g/kg/day to about 1 g/kg/day.

Embodiment 31A. The method of any one of embodiments 18A-29A, wherein the therapeutically effective amount is from about 0.8 g/kg/day.

Embodiment 32A. The method of any one of embodiments 1A-31A, wherein the quantifying comprises analysis by a plurality of assays.

Embodiment 33A. The method of any one of embodiments 1A-32A, wherein the quantifying comprises analysis by immunohistochemistry, immunofluorescence, proteomic mass spectrometry, western blot, enzyme-linked immunosorbent assay (ELISA), a genetic reporter, reverse transcription polymerase chain reaction (RT-PCR), quantitative RT-PCR (QPCR), digital PCR (dPCR), in-situ hybridization, RNA-Seq, Sanger sequencing, high throughput sequencing, or microarray.

Embodiment 34A. The method of any one of embodiments 1A-33A, wherein the quantifying is by immunohistochemistry.

Embodiment 35A. The method of any one of embodiments 1A-34A, further comprising administering a cancer therapy to the subject.

Embodiment 36A. The method of embodiment 35A, wherein the cancer therapy is radiotherapy.

Embodiment 37A. The method of embodiment 35A, wherein the cancer therapy is chemotherapy.

Embodiment 38A. The method of any one of embodiments 1A-37A, further comprising quantifying an amount of a second biosynthetic metabolic enzyme.

Embodiment 39A. The method of any one of embodiments 1A-37A, further comprising quantifying an amount of a second biosynthetic metabolic enzyme and an amount of a third biosynthetic metabolic enzyme.

Embodiment 40A. The method of any one of embodiments 1A-39A, further comprising, after the quantifying and before the administering, comparing the amount of the biosynthetic metabolic enzyme in the biological sample to a second amount of the biosynthetic metabolic enzyme in a reference biological sample, wherein the reference biological sample does not comprise a cancer cell.

Embodiment 41A. The method of embodiment 40A, wherein the amount of the biosynthetic metabolic enzyme in the biological sample is lower than the second amount of the biosynthetic metabolic enzyme in the reference sample.

Embodiment 42A. The method of any one of embodiments 1A-39A, further comprising, after the quantifying and before the administering, subjecting the biosynthetic metabolic enzyme to an empirical scoring system, wherein the empirical scoring system has a threshold value, wherein the biological sample having a sample value below the threshold value indicates a likelihood of the subject displaying a positive response to the nutrient modulation therapy.

Embodiment 43A. The method of embodiment 42A, wherein the sample value is lower than the threshold value of the empirical scoring system.

Embodiment 44A. A method comprising: a) isolating a biological sample from a subject, wherein the subject has a cancer; and b) after the isolating, quantifying an amount of a biosynthetic metabolic enzyme, wherein the amount of the biosynthetic metabolic enzyme predicts a likelihood of the subject displaying a positive response to a nutrient modulation therapy to treat the cancer.

Embodiment 45A. The method of embodiment 44A, wherein the biological sample is a tumor biopsy sample.

Embodiment 46A. The method of embodiment 44A, wherein the biological sample comprises a circulating tumor cell.

Embodiment 47A. The method of embodiment 44A, wherein the biological sample comprises circulating tumor DNA.

Embodiment 48A. The method of any one of embodiments 44A-47A, wherein the subject is human.

Embodiment 49A. The method of any one of embodiments 44A-48A, wherein the cancer is pancreatic cancer.

Embodiment 50A. The method of any one of embodiments 44A-48A, wherein the cancer is breast cancer.

Embodiment 51A. The method of any one of embodiments 44A-48A, wherein the cancer is colon cancer.

Embodiment 52A. The method of any one of embodiments 44A-51A, wherein the biosynthetic metabolic enzyme is involved in a nutrient biosynthesis.

Embodiment 53A. The method of any one of embodiments 44A-52A, wherein the biosynthetic metabolic enzyme is involved in a nutrient processing.

Embodiment 54A. The method of embodiment 52A or 53A, wherein the nutrient is a fatty acid.

Embodiment 55A. The method of embodiment 52A or 53A, wherein the nutrient is a lipid.

Embodiment 56A. The method of embodiment 52A or 53A, wherein the nutrient is an amino acid.

Embodiment 57A. The method of embodiment 56A, wherein the amino acid is serine.

Embodiment 58A. The method of embodiment 56A, wherein the amino acid is glycine.

Embodiment 59A. The method of embodiment 44A, wherein the biosynthetic metabolic enzyme is PHGDH.

Embodiment 60A. The method of embodiment 44A, wherein the biosynthetic metabolic enzyme is PSAT1.

Embodiment 61A. The method of embodiment 44A, further comprising, after the quantifying, administering a nutrient modulation therapy to the subject.

Embodiment 62A. The method of embodiment 61A, wherein the nutrient modulation therapy comprises administering a therapeutically effective amount of a dietary product.

Embodiment 63A. The method of embodiment 62A, wherein the dietary product is devoid of at least one amino acid.

Embodiment 64A. The method of embodiment 63A, wherein the dietary product is devoid of serine.

Embodiment 65A. The method of embodiment 63A, wherein the dietary product is devoid of glycine.

Embodiment 66A. The method of embodiment 63A, wherein the dietary product is devoid of proline.

Embodiment 67A. The method of embodiment 63A, wherein the dietary product is devoid of tyrosine.

Embodiment 68A. The method of embodiment 62A, wherein the dietary product is devoid of at least two amino acids.

Embodiment 69A. The method of embodiment 68A, wherein the dietary product is devoid of serine and glycine.

Embodiment 70A. The method of embodiment 63A, wherein the dietary product is devoid of at least three amino acids.

Embodiment 71A. The method of embodiment 70A, wherein the dietary product is devoid of serine, glycine, and proline.

Embodiment 72A. The method of embodiment 70A, wherein the dietary product is devoid of serine, glycine, and cysteine.

Embodiment 73A. The method of any one of embodiments 62A-72A, wherein the administering is oral.

Embodiment 74A. The method of any one of embodiments 62A-73A, wherein the therapeutically effective amount is from about 0.5 g/kg/day to about 1 g/kg/day.

Embodiment 75A. The method of any one of embodiments 62A-73A, wherein the therapeutically effective amount is from about 0.8 g/kg/day.

Embodiment 76A. The method of any one of embodiments 44A-75A, wherein the quantifying comprises analysis by a plurality of assays.

Embodiment 77A. The method of any one of embodiments 44A-76A, wherein the quantifying comprises analysis by immunohistochemistry, immunofluorescence, proteomic mass spectrometry, western blot, enzyme-linked immunosorbent assay (ELISA), a genetic reporter, reverse transcription polymerase chain reaction (RT-PCR), quantitative RT-PCR (QPCR), digital PCR (dPCR), in-situ hybridization, RNA-Seq, Sanger sequencing, high throughput sequencing, or microarray.

Embodiment 78A. The method of any one of embodiments 44A-77A, wherein the quantifying is by immunohistochemistry.

Embodiment 79A. The method of any one of embodiments 44A-78A, further comprising, after the quantifying, administering a cancer therapy to the subject.

Embodiment 80A. The method of embodiment 79A, wherein the cancer therapy is radiotherapy.

Embodiment 81A. The method of embodiment 79A, wherein the cancer therapy is chemotherapy.

Embodiment 82A. The method of any one of embodiments 44A-81A, further comprising, after the quantifying, comparing the amount of the biosynthetic metabolic enzyme in the biological sample to a second amount of the biosynthetic metabolic enzyme in a reference biological sample, wherein the reference biological sample does not comprise a cancer cell.

Embodiment 83A. The method of embodiment 82A, wherein the amount of the biosynthetic metabolic enzyme in the biological sample is lower than the second amount of the biosynthetic metabolic enzyme in the reference sample.

Embodiment 84A. The method of embodiment 44A, further comprising, after the quantifying, subjecting the amount of the first biosynthetic metabolic enzyme to an empirical scoring system, wherein the empirical scoring system has a threshold value, wherein the biological sample having a sample value below the threshold value indicates a likelihood of the subject displaying a positive response to a nutrient modulation therapy.

Embodiment 85A. The method of embodiment 84A, wherein the sample value is lower than the threshold value of the empirical scoring system.

Embodiment 86A. The method of any one of embodiments 44A-85A, further comprising, after the isolating, quantifying an amount of a second biosynthetic metabolic enzyme.

Embodiment 87A. The method of any one of embodiments 44A-85A, further comprising, after the isolating, quantifying an amount of a second biosynthetic metabolic enzyme and a third biosynthetic metabolic enzyme.

Embodiment 1B. A method of treating a cancer in a subject in need thereof, the method comprising: a) administering a therapeutically effective amount of a dietary product, wherein the dietary product is devoid of at least serine and glycine; and b) administering a therapeutically effective amount of a therapeutic agent, wherein a cancer cell of the subject expresses low levels of at least one biosynthetic metabolic enzyme, wherein the at least one biosynthetic metabolic enzyme catalyzes the biosynthesis of serine or glycine, and wherein the administering the dietary product increases an efficacy of the therapeutic agent by at least about 20% as compared to an efficacy of the therapeutic agent in the absence of the dietary product.

Embodiment 2B. The method of embodiment 1B, wherein the cancer is pancreatic cancer.

Embodiment 3B. The method of embodiment 1B, wherein the cancer is metastatic pancreatic cancer.

Embodiment 4B. The method of embodiment 1B, wherein the cancer is breast cancer.

Embodiment 5B. The method of embodiment 1B, wherein the cancer is ovarian cancer.

Embodiment 6B. The method of embodiment 1B, wherein the cancer is colon cancer.

Embodiment 7B. The method of embodiment 1B, wherein the cancer is rectal cancer.

Embodiment 8B. The method of embodiment 1B, wherein the cancer is colorectal cancer.

Embodiment 9B. The method of embodiment 1B, wherein the cancer is metastatic colorectal cancer.

Embodiment 10B. The method of any one of embodiments 1B-9B, wherein the subject is human.

Embodiment 11B. The method of any one of embodiments 1B-10B, wherein the administering of the dietary product is oral.

Embodiment 12B. The method of any one of embodiments 1B-10B, wherein the administering of the dietary product is intravenous.

Embodiment 13B. The method of any one of embodiments 1B-12B, wherein the therapeutically effective amount of the dietary product is from about 0.5 g/kg/day to about 1 g/kg/day.

Embodiment 14B. The method of any one of embodiments 1B-13B, wherein the therapeutically effective amount of the dietary product is about 0.8 g/kg/day.

Embodiment 15B. The method of any one of embodiments 1B-14B, wherein the dietary product is further devoid of proline.

Embodiment 16B. The method of any one of embodiments 1B-15B, wherein the dietary product is further devoid of tyrosine.

Embodiment 17B. The method of any one of embodiments 1B-16B, wherein the dietary product is further devoid of cysteine.

Embodiment 18B. The method of any one of embodiments 1B-17B, wherein the dietary product is further devoid of arginine.

Embodiment 19B. The method of any one of embodiments 1B-18B, wherein the dietary product is further devoid of glutamine.

Embodiment 20B. The method of any one of embodiments 1B-19B, wherein the dietary product is further devoid of glutamic acid or glutamate.

Embodiment 21B. The method of any one of embodiments 1B-20B, wherein the dietary product is formulated as a solid.

Embodiment 22B. The method of any one of embodiments 1B-20B, wherein the dietary product is formulated as a beverage.

Embodiment 23B. The method of any one of embodiments 1B-22B, wherein the dietary product is administered with food.

Embodiment 24B. The method of any one of embodiments 1B-23B, wherein the dietary product is administered 3 times a day.

Embodiment 25B. The method of any one of embodiments 1B-23B, wherein the dietary product is administered 5 times a day.

Embodiment 26B. The method of any one of embodiments 1B-25B, wherein the administering of the therapeutic agent is oral.

Embodiment 27B. The method of any one of embodiments 1B-25B, wherein the administering of the therapeutic agent is intravenous.

Embodiment 28B. The method of any one of embodiments 1B-27B, wherein the therapeutic agent is a chemotherapeutic agent.

Embodiment 29B. The method of embodiment 28B, wherein the therapeutic agent is gemcitabine.

Embodiment 30B. The method of embodiment 29B, wherein the therapeutically effective amount of gemcitabine is about 1000 mg/m².

Embodiment 31B. The method of embodiment 29B, wherein the therapeutically effective amount of gemcitabine is less than about 1000 mg/m².

Embodiment 32B. The method of embodiment 28B, wherein the therapeutic agent is paclitaxel.

Embodiment 33B. The method of embodiment 32B, wherein the therapeutically effective amount of paclitaxel is about 175 mg/m².

Embodiment 34B. The method of embodiment 32B, wherein the therapeutically effective amount of paclitaxel is less than about 175 mg/m².

Embodiment 35B. The method of any one of embodiments 1B-27B, wherein the therapeutic agent is disulfiram.

Embodiment 36B. The method of embodiment 35B, wherein the therapeutically effective amount of disulfiram is about 125 mg to about 500 mg.

Embodiment 37B. The method of embodiment 35B, wherein the therapeutically effective amount of disulfiram is less than about 250 mg.

Embodiment 38B. The method of any one of embodiments 1B-27B, wherein the therapeutic agent is a PI3K inhibitor.

Embodiment 39B. The method of embodiment 38B, wherein the PI3K inhibitor is serabelisib.

Embodiment 40B. The method of embodiment 39B, wherein the therapeutically effective amount of serabelisib is about 30 mg/kg to about 150 mg/kg.

Embodiment 41B. The method of embodiment 39B, wherein the therapeutically effective amount of serabelisib is less than about 60 mg/kg.

Embodiment 42B. The method of any one of embodiments 1B-27B, wherein the therapeutic agent is FOLFIRINOX.

Embodiment 43B. The method of any one of embodiments 1B-27B, wherein the therapeutic agent is FOLFIRI.

Embodiment 44B. The method of any one of embodiments 1B-27B, wherein the therapeutic agent is bevacizumab.

Embodiment 45B. The method of any one of embodiments 1B-27B, wherein the method comprises administering two therapeutic agents.

Embodiment 46B. The method of embodiment 45B, wherein the two therapeutic agents are each independently chemotherapeutic agents.

Embodiment 47B. The method of embodiment 46B, wherein the two therapeutic agents are gemcitabine and paclitaxel.

Embodiment 48B. The method of any one of embodiments 1B-27B, wherein the method comprises administering at least three therapeutic agents.

Embodiment 49B. The method of embodiment 48B, wherein the three therapeutic agents are folinic acid or a pharmaceutically acceptable salt thereof, 5-fluorouracil or a pharmaceutically acceptable salt thereof, and irinotecan or a pharmaceutically acceptable salt thereof.

Embodiment 50B. The method of any one of embodiments 1B-27B, wherein the method comprises administering four therapeutic agents.

Embodiment 51B. The method of embodiment 50B, wherein the four therapeutic agents are folinic acid or a pharmaceutically acceptable salt thereof, 5-fluorouracil or a pharmaceutically acceptable salt thereof, oxaliplatin, and irinotecan or a pharmaceutically acceptable salt thereof.

Embodiment 52B. The method of embodiment 50B, wherein the four therapeutic agents are folinic acid or a pharmaceutically acceptable salt thereof, 5-fluorouracil or a pharmaceutically acceptable salt thereof, irinotecan or a pharmaceutically acceptable salt thereof, and bevacizumab.

Embodiment 53B. The method of any one of embodiments 1B-52B, wherein the at least one biosynthetic metabolic enzyme catalyzes the biosynthesis of serine.

Embodiment 54B. The method of any one of embodiments 1B-53B, wherein the at least one biosynthetic metabolic enzyme is phosphoglycerate dehydrogenase (PHGDH).

Embodiment 55B. The method of embodiment 54B, wherein the subject expresses PHGDH at a level that is lower than a PHGDH level of a reference sample, wherein the reference sample does not comprise a cancer cell.

Embodiment 56B. The method of any one of embodiments 1B-53B, wherein the at least one biosynthetic metabolic enzyme is phosphoserine aminotransferase 1 (PSAT1).

Embodiment 57B. The method of embodiment 56B, wherein the subject expresses PSAT1 at a level that is lower than a PSAT1 level of a reference sample, wherein the reference sample does not comprise a cancer cell.

Embodiment 58B. The method of any one of embodiments 1B-53B, wherein the at least one biosynthetic metabolic enzyme is phosphoserine phosphatase (PSPH).

Embodiment 59B. The method of embodiment 58B, wherein the subject expresses PSPH at a level that is lower than a PSPH level of a reference sample, wherein the reference sample does not comprise a cancer cell.

Embodiment 60B. The method of any one of embodiments 1B-53B, wherein the at least one biosynthetic metabolic enzyme catalyzes the biosynthesis of glycine.

Embodiment 61B. The method of embodiment 60B, wherein the at least one biosynthetic metabolic enzyme is serine hydroxy methyltransferase 1 (SHMT1).

Embodiment 62B. The method of embodiment 61B, wherein the subject expresses SHMT1 at a level that is lower than a SHMT1 level of a reference sample, wherein the reference sample does not comprise a cancer cell.

Embodiment 63B. The method of embodiment 60B, wherein the at least one biosynthetic metabolic enzyme is serine hydroxy methyltransferase 2 (SHMT2).

Embodiment 64B. The method of embodiment 63B, wherein the subject expresses SHMT2 at a level that is lower than a SHMT2 level of a reference sample, wherein the reference sample does not comprise a cancer cell.

Embodiment 65B. The method of embodiment 60B, wherein the at least one biosynthetic metabolic enzyme is glycine synthase.

Embodiment 66B. The method of embodiment 65B, wherein the subject expresses glycine synthase at a level that is less than a glycine synthase level of a reference sample, wherein the reference sample does not comprise a cancer cell.

Embodiment 67B. The method of any one of embodiments 1B-66B, further comprising administering a ketogenic diet.

Embodiment 68B. The method of any one of embodiments 1B-67B, wherein the dose of the therapeutic agent in the presence of the dietary product is at least about 20% lower as compared to the dose of the therapeutic agent in the absence of the dietary product.

Embodiment 69B. The method of any one of embodiments 1B-67B, the dose of the therapeutic agent in the presence of the dietary product is at least about 40% lower as compared to the dose of the therapeutic agent in the absence of the dietary product.

Embodiment 70B. The method of any one of embodiments 1B-69BB, wherein the method decreases cancer cell proliferation in the subject by at least about 20% compared to a subject that is not administered the dietary product.

Embodiment 71B. The method of embodiment 70B, wherein the method decreases cancer cell proliferation in the subject by at least about 40% compared to the subject that is not administered the dietary product.

Embodiment 72B. The method of any one of embodiments 1B-71B, wherein the method decreases a tumor volume by at least about 20% compared to a subject that is not administered the dietary product.

Embodiment 73B. The method of embodiment 72B, wherein the method decreases the tumor volume by at least about 40% compared to the subject that is not administered the dietary product.

Embodiment 74B. The method of any one of embodiments 1B-73B, wherein the method decreases circulating levels of the at least two amino acids by at least about 20% compared to a subject that is not administered the dietary product.

Embodiment 75B. The method of embodiment 74B, wherein the method decreases circulating levels of the at least two amino acids by at least about 40% compared to the subject that is not administered the dietary product.

Embodiment 76B. The method of any one of embodiments 1B-75B, wherein the method decreases tissue levels of the at least two amino acids by at least about 20% compared to a subject that is not administered the dietary product.

Embodiment 77B. The method of embodiment 76B, wherein the method decreases tissue levels of the at least two amino acids by at least about 40% compared to the subject that is not administered the dietary product.

Embodiment 78B. The method of any one of embodiments 1B-77B, wherein the administering the dietary product increases the efficacy of the therapeutic agent by at least about 40% as compared to the efficacy of the therapeutic agent in the absence of the dietary product.

Embodiment 79B. The method of any one of embodiments 1B-78B, comprising administering the dietary product for a first period of time, and withholding the dietary product for a second period of time.

Embodiment 80B. A method of treating a condition in a subject in need thereof, the method comprising administering a therapeutically effective amount of a dietary product, wherein the dietary product is devoid of at least serine, glycine, and proline.

Embodiment 81B. The method of embodiment 80B, wherein the condition is a cancer.

Embodiment 82B. The method of embodiment 81B, wherein the cancer is pancreatic cancer.

Embodiment 83B. The method of embodiment 81B, wherein the cancer is metastatic pancreatic cancer.

Embodiment 84B. The method of embodiment 81B, wherein the cancer is breast cancer.

Embodiment 85B. The method of embodiment 81B, wherein the cancer is ovarian cancer.

Embodiment 86B. The method of embodiment 81B, wherein the cancer is colon cancer.

Embodiment 87B. The method of embodiment 81B, wherein the cancer is rectal cancer.

Embodiment 88B. The method of embodiment 81B, wherein the cancer is colorectal cancer.

Embodiment 89B. The method of embodiment 81B, wherein the cancer is metastatic colorectal cancer.

Embodiment 90B. The method of any one of embodiments 80B-89B, wherein the subject is human.

Embodiment 91B. The method of any one of embodiments 80B-90B, wherein the administering is oral.

Embodiment 92B. The method of any one of embodiments 80B-90B, wherein the administering is intravenous.

Embodiment 93B. The method of any one of embodiments 80B-92B, wherein the therapeutically effective amount is from about 0.5 g/kg/day to about 1 g/kg/day.

Embodiment 94B. The method of any one of embodiments 80B-92B, wherein the therapeutically effective amount is about 0.8 g/kg/day.

Embodiment 95B. The method of any one of embodiments 80B-94B, wherein the dietary product comprises at least 12 amino acids.

Embodiment 96B. The method of any one of embodiments 80B-95B, wherein the dietary product is further devoid of tyrosine.

Embodiment 97B. The method of any one of embodiments 80B-96B, wherein the dietary product is further devoid of cysteine.

Embodiment 98B. The method of any one of embodiments 80B-97B, wherein the dietary product is further devoid of arginine.

Embodiment 99B. The method of any one of embodiments 80B-98B, wherein the dietary product is formulated as a solid.

Embodiment 100B. The method of any one of embodiments 80B-98B, wherein the dietary product is formulated as a beverage.

Embodiment 101B. The method of any one of embodiments 80B-100B, wherein the dietary product is administered with food.

Embodiment 102B. The method of any one of embodiments 80B-101B, wherein the dietary product is administered 3 times a day.

Embodiment 103B. The method of any one of embodiments 80B-101B, wherein the dietary product is administered 5 times a day.

Embodiment 104B. The method of any one of embodiments 80B-103B, further comprising administering a therapeutically effective amount of a therapeutic agent.

Embodiment 105B. The method of embodiment 104B, wherein the administering the therapeutic agent is oral.

Embodiment 106B. The method of embodiment 104B, wherein the administering the therapeutic agent is intravenous.

Embodiment 107B. The method of any one of embodiments 104B-106B, wherein the therapeutic agent is a chemotherapeutic agent.

Embodiment 108B. The method of any one of embodiments 104B-107B, wherein the therapeutic agent is gemcitabine.

Embodiment 109B. The method of embodiment 108B, wherein the therapeutically effective amount of gemcitabine is about 1000 mg/m².

Embodiment 110B. The method of embodiment 108B, wherein the therapeutically effective amount of gemcitabine is less than about 1000 mg/m².

Embodiment 111B. The method of any one of embodiments 104B-107B, wherein the therapeutic agent is paclitaxel.

Embodiment 112B. The method of embodiment 111B, wherein the therapeutically effective amount of paclitaxel is about 175 mg/m².

Embodiment 113B. The method of embodiment 111B, wherein the therapeutically effective amount of paclitaxel is less than about 175 mg/m².

Embodiment 114B. The method of any one of embodiments 104B-107B, wherein the therapeutic agent is disulfiram.

Embodiment 115B. The method of embodiment 114B, wherein the therapeutically effective amount of disulfiram is about 125 mg to about 500 mg.

Embodiment 116B. The method of embodiment 114B, wherein the therapeutically effective amount of disulfiram is less than about 250 mg.

Embodiment 117B. The method of any one of embodiments 104B-107B, wherein the therapeutic agent is a PI3K inhibitor.

Embodiment 118B. The method of embodiment 117B, wherein the PI3K inhibitor is serabelisib.

Embodiment 119B. The method of embodiment 118B, wherein the therapeutically effective amount of serabelisib is about 30 mg/kg to about 150 mg/kg.

Embodiment 120B. The method of embodiment 118B, wherein the therapeutically effective amount of serabelisib is less than about 60 mg/kg.

Embodiment 121B. The method of any one of embodiments 104B-107B, wherein the therapeutic agent is FOLFIRINOX.

Embodiment 122B. The method of any one of embodiments 104B-107B, wherein the therapeutic agent is FOLFIRI.

Embodiment 123B. The method of any one of embodiments 104B-107B, wherein the therapeutic agent is bevacizumab.

Embodiment 124B. The method of any one of embodiments 80B-123B, wherein the method further comprises administering two therapeutic agents.

Embodiment 125B. The method of embodiment 124B, wherein the two therapeutic agents are each independently chemotherapeutic agents.

Embodiment 126B. The method of embodiment 124B, wherein the two therapeutic agents are gemcitabine and paclitaxel.

Embodiment 127B. The method of any one of embodiments 80B-123B, wherein the method further comprises administering at least three therapeutic agents.

Embodiment 128B. The method of embodiment 127B, wherein the three therapeutic agents are folinic acid or a pharmaceutically acceptable salt thereof, 5-fluorouracil or a pharmaceutically acceptable salt thereof, and irinotecan or a pharmaceutically acceptable salt thereof.

Embodiment 129B. The method of any one of embodiments 80B-123B, wherein the method further comprises administering four therapeutic agents.

Embodiment 130B. The method of embodiment 129B, wherein the four therapeutic agents are folinic acid or a pharmaceutically acceptable salt thereof, 5-fluorouracil or a pharmaceutically acceptable salt thereof, oxaliplatin, and irinotecan or a pharmaceutically acceptable salt thereof.

Embodiment 131B. The method of embodiment 129B, wherein the four therapeutic agents are folinic acid or a pharmaceutically acceptable salt thereof, 5-fluorouracil or a pharmaceutically acceptable salt thereof, irinotecan or a pharmaceutically acceptable salt thereof, and bevacizumab.

Embodiment 132B. The method of any one of embodiments 80B-131B, further comprising administering a ketogenic diet.

Embodiment 133B. A pharmaceutical composition comprising in unit dosage form: a) a plurality of amino acids, wherein the composition is devoid of at least serine, glycine, and proline; and b) a pharmaceutically acceptable excipient.

Embodiment 134B. The composition of embodiment 133B, wherein the composition comprises at least 12 amino acids.

Embodiment 135B. The composition of embodiment 133B or 134B, wherein the composition is further devoid of cysteine.

Embodiment 136B. The composition of any one of embodiments 133B-135B, wherein the composition is further devoid of tyrosine.

Embodiment 137B. The composition of any one of embodiments 133B-136B, wherein the composition is further devoid of asparagine.

Embodiment 138B. The composition of any one of embodiments 133B-137B, wherein the unit dosage form has a weight of about 12 grams.

Embodiment 139B. The composition of any one of embodiments 133B-138B, wherein the composition is in a powder form.

Embodiment 140B. The composition of any one of embodiments 133B-139B, wherein the composition further comprises from about 2% to about 5% of a K⁺ source.

Embodiment 141B. The composition of embodiment 140B, wherein the K⁺ source comprises potassium citrate, potassium phosphate, potassium chloride, potassium sulfate, potassium gluconate, potassium bicarbonate, potassium aspartate, potassium acetate, and potassium orotate.

Embodiment 142B. The composition of any one of embodiments 133B-141B, wherein the composition further comprises from about 1% to about 4% (w/w) of a Mg²⁺ source.

Embodiment 143B. The composition of embodiment 142B, wherein the Mg²⁺ source comprises magnesium citrate, magnesium fumarate, magnesium acetate, magnesium aspartate, magnesium threonate, magnesium glycinate, magnesium chloride, magnesium sulfate, magnesium oxide, or magnesium malate, magnesium orotate, or a hydrate thereof.

Embodiment 144B. The composition of any one of embodiments 133B-143B, wherein the composition further comprises taurine, a hydrate thereof, or a salt thereof from about 0.05% to about 2% (w/w).

1-43. (canceled)

44. A method for identifying a subject for treatment of adrenal, blood, breast, colorectal, endometrial, kidney, liver, lymphocytic, pancreatic, stomach, or thyroid cancer comprising:
quantifying an amount of a metabolic enzyme from an isolated biological sample from the subject,
wherein the amount of the metabolic enzyme predicts a successful response of the subject to a nutrient modulation therapy to treat the cancer, and
wherein the metabolic enzyme is involved in fatty acid, lipid, or amino acid biosynthesis.

45. The method of claim 44, wherein the biological sample is a tumor biopsy sample.

46. The method of claim 44, wherein the biological sample comprises a circulating tumor cell.

47. The method of claim 44, wherein the biological sample comprises circulating tumor DNA.

48. (canceled)

49. The method of claim 44, wherein the cancer is pancreatic cancer.

50. The method of claim 44, wherein the cancer is breast cancer.

51. The method of claim 44, wherein the cancer is colorectal cancer.

52-53. (canceled)

54. The method of claim 44, wherein the metabolic enzyme is involved in is a fatty acid biosynthesis.

55. The method of claim 44, wherein the metabolic enzyme is involved in is a lipid biosynthesis.

56. The method of claim 44, wherein the metabolic enzyme is involved in amino acid synthesis.

57. The method of claim 56, wherein the biosynthetic metabolic enzyme is involved in serine biosynthesis.

58. The method of claim 56, wherein the biosynthetic metabolic enzyme is involved in glycine biosynthesis.

59. The method of claim 44, wherein the metabolic enzyme is PHGDH.

60. The method of claim 44, wherein the metabolic enzyme is PSAT1.

61. The method of claim 44, further comprising, after the quantifying, administering a nutrient modulation therapy to the subject.

62. The method of claim 61, wherein the nutrient modulation therapy comprises administering a therapeutically effective amount of a dietary product devoid of serine and glycine.

63-70. (canceled)

71. The method of claim 61, wherein the nutrient modulation therapy comprises administering a therapeutically effective amount of a dietary product devoid of serine, glycine, and proline.

72. The method of claim 61, wherein the nutrient modulation therapy comprises administering a therapeutically effective amount of a dietary product devoid of serine, glycine, and cysteine.

73-76. (canceled)

77. The method of claim 44, wherein the quantifying comprises analysis by immunohistochemistry, immunofluorescence, proteomic mass spectrometry, western blot, enzyme-linked immunosorbent assay (ELISA), a genetic reporter, reverse transcription polymerase chain reaction (RT-PCR), quantitative RT-PCR (QPCR), digital PCR (dPCR), in-situ hybridization, RNA-Seq, Sanger sequencing, high throughput sequencing, or microarray.

78. The method of claim 77, wherein the quantifying is by immunohistochemistry.

79. The method of claim 61, further comprising, after the quantifying, administering radiotherapy or chemotherapy to the subject.

80-81. (canceled)

82. The method of claim 56, further comprising, after the quantifying, comparing the amount of the metabolic enzyme in the biological sample to a second amount of the metabolic enzyme in a reference biological sample, wherein the reference biological sample does not comprise a cancer cell.

83. The method of claim 82, wherein the amount of the metabolic enzyme in the biological sample is lower than the second amount of the metabolic enzyme in the reference sample.

84. The method of claim 56, further comprising, after the quantifying, subjecting the amount of the metabolic enzyme to an empirical scoring system, wherein the empirical scoring system has a threshold value, wherein the biological sample having a sample value below the threshold value indicates a likelihood of the subject displaying a positive response to a nutrient modulation therapy.

85. The method of claim 84, wherein the sample value is lower than the threshold value of the empirical scoring system.

86-87. (canceled)

88. The method of claim 44, wherein the metabolic enzyme is PSPH.

89. The method of claim 59, wherein the method further comprises quantifying an amount of PSPH from the isolated biological sample from the subject or from a second isolated biological sample from the subject,
wherein the amount of the PHGDH and the amount of the PSPH predict a successful response of the subject to a nutrient modulation therapy to treat the cancer.

90. The method of claim 60, wherein the method further comprises quantifying an amount of PSPH from the isolated biological sample from the subject or from a second isolated biological sample from the subject,
wherein the amount of PSAT1 and the amount of the PSPH predict a successful response of the subject to a nutrient modulation therapy to treat the cancer.

91. The method of claim 59, wherein the method further comprises quantifying an amount of PSAT1 from the isolated biological sample from the subject or from a second isolated biological sample from the subject,
wherein the amount of the PHGDH and the amount of the PSAT1 predict a successful response of the subject to a nutrient modulation therapy to treat the cancer.

92. The method of claim 91, wherein the method further comprises quantifying an amount of PSPH from the isolated biological sample from the subject, from the second isolated biological sample from the subject, or from a third isolated biological sample from the subject,
wherein the amount of PHGDH, the amount of PSAT1, and the amount of PSPH predict a successful response of the subject to a nutrient modulation therapy to treat the cancer.

93. The method of claim 56, wherein the cancer is endometrial cancer.
